(19) United States
(12) Patent Application Publication
(10) Pub. No.: US 2022/0106595 A1
(43) Pub. Date: Apr. 7, 2022

(54) ANTIBACTERIAL ANTISENSE AGENTS

(71) Applicant: Pedanius Therapeutics Limited, Macclesfield (GB)

(72) Inventors: Martin Quibell, Biohub, Alderley Park (GB); Timothy Schulz-Utermoehl, Biohub, Alderley Park (GB); Fraser Murray, Biohub, Alderley Park (GB)

(73) Assignee: Pedanius Therapeutics Limited, Macclesfield (GB)

(21) Appl. No.: 17/296,748

(22) PCT Filed: Nov. 27, 2019
(86) PCT No.: PCT/GB2019/053354

§ 371 Date: May 25, 2021

(30) Foreign Application Priority Data
Nov. 28, 2018 (GB) 1819399.5

(51) Int. Cl. C12N 15/113; A61P 31/04

(52) U.S. Cl. CPC C12N 15/113; A61P 31/04; C12N 2310/11; C12N 2320/30; C12N 2310/3233; C12N 2310/3181; C12N 2310/314

(57) ABSTRACT

The invention relates to improved ANTISENSE agents for the treatment of gram-negative bacterial infections. Compounds of the invention utilise an Antibiotic-Assisted Translocation; AAT′ platform to improve influx into bacterial cells through enhanced permeability, providing improved intracellular exposure of the ANTISENSE AGENT and superior treatment of the infection.

### FIELD OF THE INVENTION

The present invention relates to compounds that include antisense oligomers targeted against genes that contribute to virulence, antibiotic resistance, biofilm formation or essential growth and survival processes in bacterial infections, particularly gram-negative bacterial infections. The present invention relates to compounds that include antisense oligomers that are useful in the monotherapy treatment of bacterial infections or, through the use of combinations with known antibiotics, useful in imparting improved and clinically meaningful activity (minimum inhibitory concentration; MIC) against bacterial infections. Whilst not bound by a specific theory, the compounds of the invention may generate higher intracellular concentrations of the antisense oligomer in bacterial cells than can be otherwise achieved by use of the antisense oligomer alone. Compounds of the invention contain an antibiotic-assisted translocation (AAT) moiety that imparts increased influx into bacterial cells through enhanced permeability. The compounds of the present invention may improve intracellular exposure of the antisense oligomer relative to the intracellular exposure achieved by administering antisense oligomer alone. The compounds of the present invention may improve treatment of a bacterial infection relative to treatment with the antisense oligomer alone.

### BACKGROUND TO THE INVENTION

Drug-resistant bacterial infections are already responsible for a significant number of deaths globally each year and the development of new therapeutic approaches and new antibacterial drugs is becoming an increasingly urgent requirement. Of these drug-resistant infections, those caused by MDR Gram-negative pathogens such as Enterobacter species, Acinetobacter baumannii, Pseudomonas aeruginosa and Klebsiella pneumoniae are amongst the most serious health threats. Many Gram-negative bacteria are now resistant to a significant number of old and current antibiotics and can cause infections that are difficult to treat.

A new therapeutic approach to treatment of human diseases and infections is through the use of antisense oligonucleotides (ASOs) that target protein biosynthesis at the genetic level to provide the positive therapeutic endpoint. A number of products based on these principles have been approved for use, for example Fomivirsen® (an antisense antiviral drug that was used in the treatment of cytomegalovirus retinitis (CMV) in immunocompromised patients), Kynamro® (used to treat homozygous familial hypercholesterolemia), and Alicaforsen® (that targets the mRNA for the production of human ICAM-1 protein and indicated for pouchitis).

The treatment of bacterial infections through the use of ASOs that target gene products essential for bacterial growth, survival or the development of resistance mechanisms holds enormous potential (e.g. see (i) Bai, H. et al., Curr. Drug Disc. Tech., 7, 76-85, 2010; (ii) Hatamoto, M. et al., App. Microbiol. Technol., 86, 397-402, 2010; (iii) Geller, B. L. et al., J. Infec. Dis., 208, 1553-1560, 2013; (iv) Good, L. & Stach, E. M., Frontiermicrobiol., 2, 185, 2011; (v) Hansen, A. M. et al., Bioconj. Chem. 27(4), 863-7, 2016; (vi) Sully, E. K. & Geller, B. L. Curr. Opin. Microbiol., 33, 47-55, 2016; (vii) Hegarty, J. P. & Stewart Sr, D. B. Appl. Microbiol & Biotech. 102(3), 1055-65, 2018; (viii) Geller, B. L. et al., J. Antimicrob. Chemotherapy, 73, 1611-1619, 2018; (ix) Howard, J. J. et al., Antimicrob. Agents & Chemotherapy, 61(4), 2017; (x) WO2015/032968; (xi) WO2015/175977; (xii) WO2015/179249; (xiii) WO2016/108930; (xiv) WO2017/112885; (xv) WO2017/112888).

Natural oligonucleotides are rapidly broken down in the systemic circulation by endo and exo-nucleases. Therefore, the art of ASOs has evolved through a number of iterations (generations) to improve their stability to nucleases through modification of the natural oligonucleotide sugar and linkage within each monomer unit (see Scheme 1). In the field of bacterial ASOs, the PMO (phosphorodiamidate morpholino) and PNA (peptide nucleic acid) modified oligonucleotides are extensively explored.

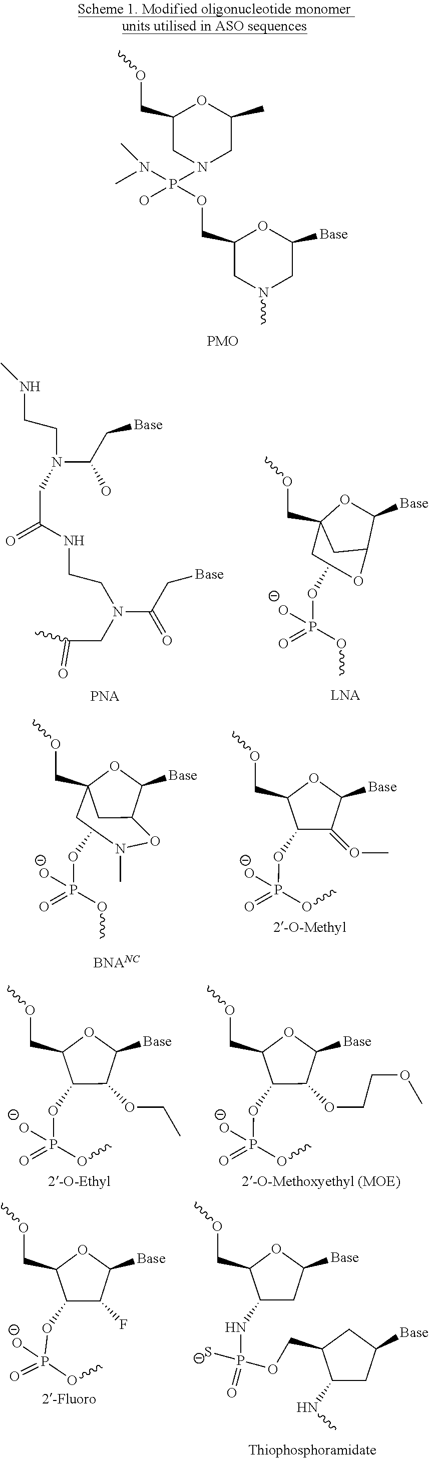

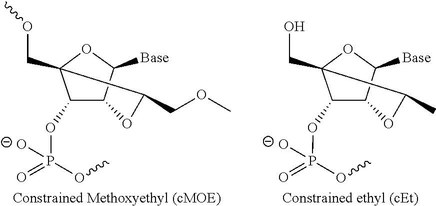

To exert their therapeutic effect, bacterial ASOs need to penetrate the bacterial membranes and transit into the cytoplasm. Unlike eukaryotic cells, bacteria have the double-strand DNA located in the bacterial nucleoid that has no nucleic membrane. RNA transcription and protein synthesis in bacteria are processed in the cytoplasm and as such antisense oligomers that reach this intracellular compartment may exert their effect. Natural and modified ASOs do not possess the physiochemical properties required to achieve this and at present virtually all intracellular delivery of ASOs requires attachment of a cell-penetrating peptide (CPP) signal. Historically, CPPs are small highly charged peptide signals (6→20+amino acids in length) with origins from HIV TAT protein or penetratin, a 16-residue peptide derived from the Drosophila Antennapedia gene (e.g. see (i) McClorey, G & Banerjee, S., Biomedicines, 6(2), E51, 2018; (ii) Shiraishi, T and Nielsen, P. E. Methods Mol. Biol., 1050, 193-205, 2014). Although effective, in general CPPs are non-discriminant and the antisense cargo that is attached to a CPP is delivered into many tissues and cells, leading to toxicity and low therapeutic windows for the disease of interest. Therefore, a mechanism of toxicity for CPP-ASOs is hybridization-dependent off-target effects in healthy cells that can potentially occur due to the binding of ASOs to complementary regions of unintended RNAs. This off-target toxicity becomes an even more important consideration as the number of complementary regions increases dramatically with tolerated mismatches (e.g. see Yoshida, T. et al., Genes Cells, 23(6), 448-455, 2018). Therefore, a method to improve the preferential delivery of bacterial ASOs into bacteria, whilst minimising intracellular exposure into healthy human cells and thereby significantly reducing the potential for unwanted off-target effects would provide a major advance to the state of the art.

### SUMMARY OF THE INVENTION

The bacterial uptake mechanism detailed herein exploits naturally occurring sugars, namely N-acetyl D-muramic acid (MurNAc) which is the ether of lactic acid and N-acetylglucosamine and is a key element in forming the backbone of the cell wall peptidoglycan of Gram-negative bacteria and a cyclic variant namely 1,6-anhydro-N-acetylmuramic acid (anhMurNAc). Chemical attachment of these sugars to the ASO either directly or indirectly via a linker provides the compounds of the present invention, herein termed an “AAT ASO” or an “AAT antisense agent”. Because the AAT antisense agent requires cytoplasm-based enzymes within the bacteria to release the parent ASO, only low levels of the parent ASO are ever present in the peripheral circulation. Also, since a CPP signal peptide is not used herein, the AAT ASOs detailed herein exhibit very limited penetration into healthy mammalian cells and therefore a dramatically improved opportunity for a beneficial toxicity profile. This is a key aspect of the invention since the most effective AAT antisense agent aims to provide intracellular exposure of the ASO primarily within the bacteria.

A bacterial antisense sequence is conjugated (i.e. chemically bonded) directly or indirectly via a linker to a sugar moiety thereby providing an AAT antisense agent. The AAT antisense agent may exhibit selective uptake across the bacterial membranes into the cytoplasm of Gram-negative bacteria. Depending upon the design of the AAT antisense agent, the parent antisense oligomer may be subsequently cleaved and released through bacterial enzymatic process(s) catalysed by a selective ligase and/or amidase. Alternatively, the full AAT ASO construct may remain intact and elicit a similar or equivalent antisense activity (e.g. see Bai, H. et alibidwherein a CPP-ASO retained full potency with respect to the parent ASO).

The AAT ASO compounds of the present invention may have intrinsic antibacterial activity when targeting genes that produce protein products that are essential for bacterial growth and survival. Alternatively, the AAT ASOs of the present invention may target bacterial genes that produce protein products that have evolved as resistance mechanisms for otherwise effective antibacterial drugs. In this example, administration of a combination of the AAT ASO and an existing antibiotic may improve the antibacterial activity of the antibiotic by concomitant antisense inhibition of the bacterial resistance mechanism.

The sugar portion of the compounds of the present invention (AAT ASO agents) may be any tautometric form of the sugar, including an open or cyclic (closed) form. It will be understood to those skilled in the art that when in solution the sugar groups exist in equilibrium between their open chain acyclic and closed cyclic forms. For instance, one sugar of interest in the present invention, N-acetyl muramic acid exists in the forms as shown in the tautomeric equilibrium (Scheme 2). Within the scope of the invention are compounds in both the open acyclic or closed cyclic form or in equilibrium between the two forms. Wherein the AAT ASO agent is shown in one form, it is intended to include the other tautomeric form as well as both open and closed forms in equilibrium.

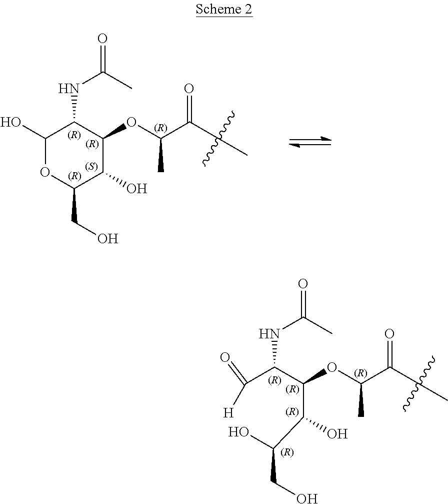

A first aspect of the invention relates to compounds of general formula (I), and pharmaceutically acceptable salts thereof,

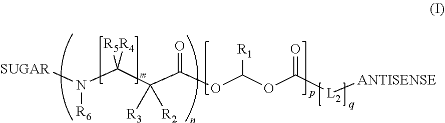

wherein,
ANTISENSE is an oligonucleotide having natural, artificial and/or modified nucleobases, the oligonucleotide selected from the group consisting of phosphodiester oligonucleotides (PDOs), phosphorothioate oligonucleotides (PSOs), phosphorodiamidate morpholino oligonucleotides (PMOs), peptide nucleic acids (PNAs), locked nucleic acids (LNAs), 2′-O-Alkyl oligonucleotides (2′-O-Me, 2′-O-Et. 2′-O-methoxyethyl) and combinations thereof; wherein the oligonucleotide is bonded to the remainder of the molecule of formula I via a terminal amino group present within the ANTISENSE sequence; and
L₂ is a spacer that forms a chemical bond to a terminal amino group present within the ANTISENSE sequence and a second chemical bond to the terminal carbonyl of the remainder of the molecule of formula I and is chosen from the group consisting of:

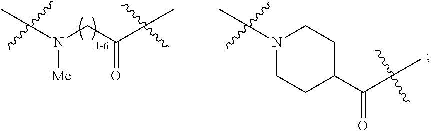

SUGAR is any tautomeric form of the acyl fragment of an N-acylmuramic acid or 1,6-anhydro-N-acylmuramic acid having the structure:

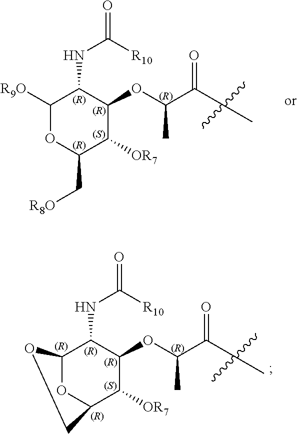

R₁ and R₆ are each independently selected from the group consisting of:
H, C₁₋₆ alkyl, C₁₋₆ substituted alkyl, C₃₋₈ cycloalkyl, C₃₋₈ substituted cycloalkyl, phenyl and benzyl;

R₂ and R₃ are each independently selected from the group consisting of:
H, C₁₋₆ alkyl, C₁₋₆ substituted alkyl, C₃₋₈ cycloalkyl, C₃₋₈ substituted cycloalkyl, phenyl and benzyl, or both together with the carbon atom to which they are attached form a ring containing 3, 4, 5 or 6 carbon atoms; and

R₄ and R₅ are each independently selected from the group consisting of:
H, C₁₋₆ alkyl, C₁₋₆ substituted alkyl, C₃₋₈ cycloalkyl, C₃₋₈ substituted cycloalkyl, phenyl and benzyl, or both together with the carbon atom to which they are attached form a ring containing 3, 4, 5 or 6 carbon atoms;

or

R₂ and R₄ together with the adjacent carbon atoms to which they are attached form a ring containing 3, 4, 5 or 6 carbon atoms; and R₃ and R₅ are each independently selected from the group consisting of: H, C₁₋₆ alkyl, C₁₋₆ substituted alkyl, C₃₋₈ cycloalkyl, C₃₋₈ substituted cycloalkyl, phenyl and benzyl, or both together with the carbon atom to which they are attached form a ring containing 3, 4, 5 or 6 carbon atoms;

R₇, R₈ and R₉ are each independently selected from the group consisting of:
H, acetyl, benzoyl; and

R₁₀ is selected from the group consisting of:
methyl, ethyl, propyl; and
m is 0 or 1 or 2; and
n is 0 or 1 or 2 or 3 or 4, wherein when n is 2, 3 or 4, each

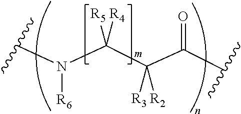

residue is independently selected; and
p is 0 or 1; and
q is 0 or 1.

A second aspect of the invention relates to a compound of the invention for use as a medicament.

A third aspect of the invention relates to a pharmaceutical or veterinary composition comprising a compound of the invention and a pharmaceutically acceptable or veterinarily acceptable diluent, excipient and/or carrier.

A fourth aspect of the invention relates to a compound of the invention for use in the treatment of bacterial infections.

A fifth aspect of the invention relates to a compound of the invention for use in the treatment of multi-drug resistant (MDR) bacterial infections.

A sixth aspect of the invention relates to a compound of the invention for use in the treatment of gram-negative bacterial infections. Such gram-negative bacterial infections may be multi-drug resistant (MDR) gram-negative bacterial infections.

A seventh aspect of the invention relates to a compound of the invention for use as a therapeutic in combination with any other antibiotic.

An eighth aspect of the invention relates to a method of treating bacterial infections that involves administering to a subject in need thereof a therapeutically effective amount of a compound of the invention.

A ninth aspect of the invention relates to a method of treating multi-drug resistant (MDR) bacterial infections that involves administering to a subject in need thereof a therapeutically effective amount of a compound of the invention.

A tenth aspect of the invention relates to a method of treating gram-negative bacterial infections that involves administering to a subject in need thereof a therapeutically effective amount of a compound of the invention. Such gram-negative bacterial infections may be multi-drug resistant (MDR) gram-negative bacterial infections.

An eleventh aspect of the invention relates to a method of reducing the adverse side-effects associated with systemic exposure to an antisense oligonucleotide through use of a compound of the invention to target preferential accumulation of the antisense oligonucleotide within multi-drug resistant (MDR) bacteria, e.g. within MDR gram-negative bacteria.

A twelfth aspect of the invention provides a method comprising intravenous administration to a subject of a therapeutically effective amount of a compound of the invention.

A thirteenth aspect of the invention relates to intravenous administration of compounds of the invention providing direct distribution to bacteria-infected tissues prior to passage and metabolism in the hepatic circulation.

A fourteenth aspect of the invention provides preferential accumulation of compounds of the invention in gram-negative pathogen infected cells when compared to other mammalian cells and tissues.

A fifteenth aspect of the invention relates to the use of a compound according to the invention in combination with an existing antibiotic towards an advantageous change in the optimal pharmacokinetic-pharmacodynamic relationship that is otherwise observed for the existing antibiotic.

### DESCRIPTION OF THE FIGURES

FIG. 1: Scatter plot of Log₁₀ CFU/g bladder of E. coli (ATCC25922) in mice following treatment with reference and test compounds. Vertical and horizontal bars represent SD and mean, respectively.

FIG. 2: Scatter plot of Log₁₀ CFU/g kidneys (pool of left and right kidney) of E. coli (ATCC25922) in mice following treatment with reference and test compounds. Vertical and horizontal bars represent SD and mean, respectively.

FIG. 3: Scatter plot of Log₁₀ CFU/g bladder of E. coli (CFT073, ATCC®700928™) in mice following treatment with reference and test compounds. Vertical and horizontal bars represent SD and mean, respectively.

FIG. 4: Scatter plot of Log₁₀ CFU/g kidneys (pool of left and right kidney) of E. coli (CFT073, ATCC®700928™) in mice following treatment with reference and test compounds. Vertical and horizontal bars represent SD and mean, respectively.

FIG. 5: Scatter plot of Log₁₀ CFU/g bladder of E. coli (ATCC BAA-2340) in mice following treatment with reference and test compounds. Vertical and horizontal bars represent SD and mean, respectively.

FIG. 6: Scatter plot of Log₁₀ CFU/g kidneys (pool of left and right kidney) of E. coli (ATCC BAA-2340) in mice following treatment with reference and test compounds. Vertical and horizontal bars represent SD and mean, respectively.

FIG. 7: Scatter plot of Log₁₀ CFU/g lung of A. baumannii (ATCC19606) in mice following treatment with reference and test compounds. Vertical and horizontal bars represent SD and mean, respectively.

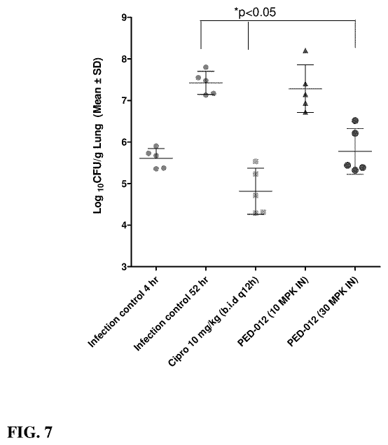

### DETAILED DESCRIPTION OF THE INVENTION

### Definitions

As used herein, the terms “ANTISENSE”, “ASO” or “oligomer” refers to a linear sequence of nucleotides, or nucleotide analogues, which allows the nucleobases (e.g. a purine or pyrimidine) to mimic the structure of nucleic acid and bind through well characterised Watson-Crick base pairing to bacterial DNA or RNA to prevent production of protein products that are essential for bacterial growth, survival or development of resistance mechanisms. The terms “ANTISENSE”, “ASO” or “oligomer” also encompass sequences that have one or more additional moieties conjugated at the 5′- or 3′-end such as a 5′-N-methylgylcinamide. Typically, the synthetic oligomers are modified sequences termed PMOs or PNAs as depicted below.

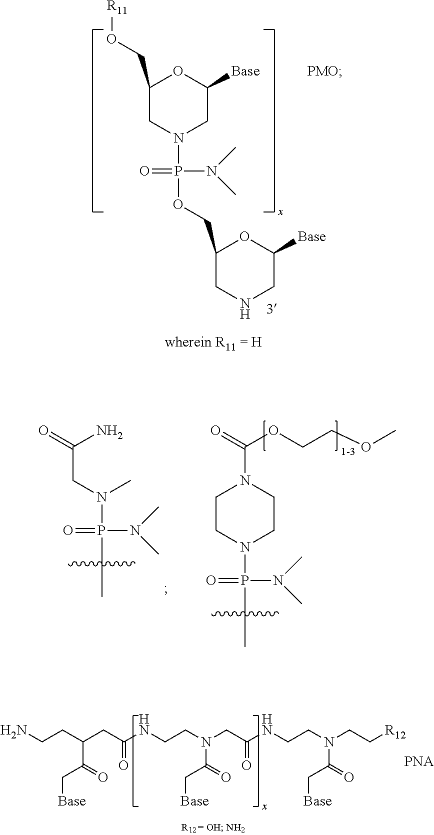

As would be clear to a person of skill in the art, the ANTISENSE sequences are presented in the tables herein in the conventional 5′ to 3′ direction. Either the 3′ end or the 5′ end of the ANTISENSE sequence can bond to the remainder of the molecule described herein (of Formula I) via a terminal amino group. For example, in the PMO structure depicted above, the morpholino group can be used to bond the ANTISENSE sequence to the remainder of the molecule described herein, and in the PNA structure depicted above, the left hand terminal amino group can be used to bond the ANTISENSE sequence to the remainder of the molecule described herein. In an embodiment, it is the 3′ end of the sequence that is bonded to the remainder of the molecule described herein (of Formula I) via the terminal amino group. However, in an alternative embodiment, it is the 5′ end of the sequence that is bonded to the remainder of the molecule described herein (of Formula I) via the terminal amino group. In order to achieve binding at the 5′ end, an ASO functionalised with a 5′-primary amine (which is commercially available) can be capped at the 3-end to avoid reaction at the 3′ end.

The following schematic depicts how a PMO can be substituted at the 3′ end or the 5′ end:

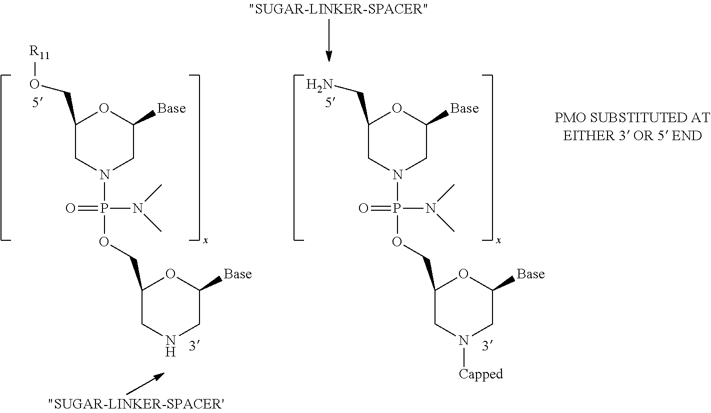

The term “antibiotic”, “antibacterial” or “antibacterial agent”, unless otherwise indicated, refers to any of the classes of compounds that have antibacterial activity against Gram-positive or Gram-negative bacteria.

The term “SPACER”, unless otherwise indicated, refers to a fragment (L₂ in formula (I)) that chemically bonds the “ANTISENSE” to the “LINKER” that in turn is bonded to the “SUGAR” such that the chemical bonds can be stable or cleaved by intracellular bacterial enzymatic processes.

The term “LINKER”, unless otherwise indicated, refers to a fragment that chemically bonds the “SUGAR” to the “SPACER” that in turn is bonded to the “ANTISENSE” such that the chemical bonds can be stable or cleaved by intracellular bacterial enzymatic processes.

The term “SUGAR”, unless otherwise indicated, refers to the acyl fragment that chemically bonds to the “LINKER” which in turn chemically bonds to “SPACER” that in turn chemically bonds to the “ANTISENSE” such that the chemical bonds can be stable or can be cleaved by intracellular bacterial enzymatic processes. Herein, the term “SUGAR” specifically refers to N-acetyl D-muramic acid (MurNAc), 1,6-anhydro-N-acetyl D-muramic acid (anhMurNAc) and the simple N-acyl variants thereof within the scope of general formula I. One or more of the functional groups within the “SUGAR” may be protected. Suitable amino-protecting groups include, for example, acetyl and azido. Suitable hydroxy-protecting groups include, for example acetyl, benzyl, benzoyl and benzylidine.

The term “SUGAR-LINKER”, unless otherwise indicated, refers to the acyl fragment that is formed by chemical bonding of the “SUGAR” and “LINKER” and in turn chemically bonds to the “SPACER-ANTISENSE” to form the full “SUGAR-LINKER-SPACER-ANTISENSE AGENT”.

The term “SUGAR-LINKER-SPACER”, unless otherwise indicated, refers to the acyl fragment that is formed by chemical bonding of the “SUGAR” and “LINKER” and “SPACER” that in turn chemically bonds to the “ANTISENSE” to form the full “SUGAR-LINKER-SPACER-ANTISENSE AGENT”.

As used herein, the term ‘alkyl’ includes stable straight and branched chain aliphatic carbon chains which may be optionally substituted. Preferred examples include methyl, ethyl, n-propyl, isopropyl, n-butyl, isobutyl, t-butyl, pentyl, isopentyl and hexyl and any simple isomers thereof. Preferably, the alkyl group is a C₁₋₄ alkyl group. Substituents for the alkyl group may be halogen, e.g. fluorine, chlorine, bromine and iodine, OH or C₁-C₄ alkoxy. Other substituents for the alkyl group may alternatively be used. Other substituents for the alkyl groups include COOH and NH₂ (and protected analogues thereof).

‘Halogen’ or ‘Halo’ as applied herein encompasses F, Cl, Br, I.

‘Heteroatom’ as applied herein encompasses O, S, P and N, more preferably, O, S and N.

As used herein, the term “cycloalkyl” refers to a cyclic alkyl group (i.e. a carbocyclic ring) which may be substituted (mono- or poly-) or unsubstituted. Substituents for the cycloalkyl group may be halogen, e.g. fluorine, chlorine, bromine and iodine, OH, C₁-C₄ alkyl or C₁-C₄ alkoxy. Other substituents for the cycloalkyl group may alternatively be used. Suitable substituents include, for example, one or more halo groups. Preferably, the cycloalkyl group is a C₃₋₆-cycloalkyl. Examples include cyclopropyl, cyclobutyl, cyclopentyl, cyclohexyl and the like. In addition, the carbocyclic ring itself may optionally contain one or more heteroatoms, for example, to give a heterocycloalkyl group such as tetrahydrofuran, pyrrolidine, piperidine, piperazine or morpholine.

Aromatic groups (e.g. phenyl and benzyl) may be optionally substituted, for example, by one or more C₁₋₆ alkoxy, OH, COOH, COOMe, NH₂, NMe₂, NHMe, NO₂, CN, CF₃ and/or halo groups.

Heteroaromatic groups may be optionally substituted, for example, by one or more C₁₋₆ alkoxy, OH, COOH, COOMe, NH₂, NMe₂, NHMe, NO₂, CN, CF₃ and/or halo groups.

The present invention includes all salts, hydrates, solvates, complexes of the compounds of this invention. The term “compound” is intended to include all such salts, hydrates, solvates, complexes and prodrugs, unless the context requires otherwise.

The present invention also includes deutero analogues of the compounds of this invention (see (a) Tung, R., “Deuterium medicinal chemistry comes of age”, Future Med. Chem., 8(5), 491-4, 2016; (b) Uttamsingh, V. et al., “Altering metabolic profiles of drugs by precision deuteration”, J. Pharmacol. Exp. Ther., 354(1), 43-54, 2015). The term “compound” is intended to also include all deutero analogues, unless the context requires otherwise.

Abbreviations and symbols commonly used in the peptide and chemical arts are used herein to describe compounds of the present invention, following the general guidelines presented by the IUPAC-IUB Joint Commission on Biochemical Nomenclature as described in Eur. J. Biochem., 158, 9-, 1984. Compounds of formula (I) and the intermediates and starting materials used in their preparation are named in accordance with the IUPAC rules of nomenclature in which the characteristic groups have decreasing priority for citation as the principle group.

The term “AAT antisense agent”, as used herein, includes but is not limited to a compound of the invention that includes the ANTISENSE sequences listed in Table 1A-Table 1D, wherein the “SUGAR-LINKER-SPACER” is covalently attached through the preferred functional groups detailed. The AAT antisense agent may be therapeutically inactive until cleaved to release the parent ANTISENSE or may retain inherent antibacterial activity of its own.

The term “parent ANTISENSE” refers to the antisense oligomer sequence of the AAT antisense agent, without the “SUGAR-LINKER-SPACER”. The terms “parent ANTISENSE”, “parent” and “parent compound” are used interchangeably herein.

Unless otherwise specified, the term “naturally occurring” refers to occurring in nature, for example, in bacteria or in a mammal (e.g., a human).

In one embodiment, the term “pharmaceutically acceptable salts” embraces salts commonly used to form alkali metal salts and to form addition salts of free acids or free bases. The nature of the salt is not critical, provided that it is pharmaceutically acceptable. Suitable pharmaceutically acceptable acid addition salts may be prepared from an inorganic acid or an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic (e.g., trifluoroacetic acid), propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, p-hydroxybutyric, salicylic, galactaric and galacturonic acid. Suitable pharmaceutically acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenedi amine, meglumine (N-methylglucamine) and procaine. These salts may be prepared, for example, by reacting, in another embodiment, the appropriate acid or base with the compound.

In one embodiment, the term “pharmaceutically acceptable carriers” includes, but is not limited to, 0.01-0.1 M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In one embodiment the level of phosphate buffer used as a pharmaceutically acceptable carrier is between about 0.01 to about 0.1M, or between about 0.01 to about 0.09M in another embodiment, or between about 0.01 to about 0.08M in another embodiment, or between about 0.01 to about 0.07M in another embodiment, or between about 0.01 to about 0.06M in another embodiment, or between about 0.01 to about 0.05M in another embodiment, or between about 0.01 to about 0.04M in another embodiment, or between about 0.01 to about 0.03M in another embodiment, or between about 0.01 to about 0.02M in another embodiment, or between about 0.01 to about 0.015 in another embodiment.

The term “systemic administration” as used herein refers to oral, sublingual, buccal, transnasal, transdermal, rectal, intramuscular, intravenous, intraventricular, intrathecal, and subcutaneous routes.

The term “intravenous administration” includes injection and other modes of intravenous administration.

The terms “administration of” or “administering a” compound refers to providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

Techniques and compositions for making useful dosage forms using the present invention are described in one or more of the following references: Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), and the like, relevant portions of each incorporated herein by reference.

The term “subject” refers to a mammal, such as humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, and cats, avian species, such as chickens, turkeys, and songbirds. Preferably, the subject is a human. The subject can be, for example, a child, such as an adolescent, or an adult.

The term “treatment” refers to any treatment of a pathologic condition in a mammal, particularly a human, and includes: (i) preventing the pathologic condition from occurring in a subject which may be predisposed to the condition but has not yet been diagnosed with the condition and, accordingly, the treatment constitutes prophylactic treatment for the disease condition; (ii) inhibiting the pathologic condition, i.e., arresting its development; (iii) relieving the pathologic condition, i.e., causing regression of the pathologic condition; or (iv) relieving the conditions mediated by the pathologic condition.

The term “therapeutically effective amount” refers to that amount of a compound of the invention that is sufficient to effect treatment, as defined above, when administered to a mammal in need of such treatment. The therapeutically effective amount will vary depending upon the subject and disease condition being treated, the weight and age of the subject, the severity of the disease condition, the manner of administration and the like, which can readily be determined by one of ordinary skill in the art.

Pharmaceutical Compositions:

The pharmaceutical composition may include one or more excipients including, but not limited to, lubricants (such as magnesium stearate, calcium stearate, zinc stearate, powdered stearic acid, hydrogenated vegetable oils, talc, polyethylene glycol, and mineral oil), colorants, binders (sucrose, lactose, gelatin, starch paste, acacia, tragacanth, povidone, polyethylene glycol, Pullulan and corn syrup), glidants (such as colloidal silicon dioxide and talc), surface active agents (such as sodium lauryl sulfate, dioctyl sodium sulfosuccinate, triethanolamine, polyoxyethylene sorbitan, poloxalkol, and quaternary ammonium salts), preservatives, stabilizers, adhesives (such as mucoadhesives), disintegrants, bulking substances, flavorings, sweeteners, pharmaceutically acceptable carriers, and other excipients (such as lactose, mannitol, glucose, fructose, xylose, galactose, sucrose, maltose, xylitol, sorbitol, chloride, sulfate and phosphate salts of potassium, sodium, and magnesium).

The AAT antisense agents of the invention may be formulated into an oral dosage forms (such as tablets and capsules) by methods known in the art. Examples of dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, troches, lozenges, chewable lozenges, beads, powders, granules, particles, microparticles, dispersible granules, cachets, thin strips, oral films, transdermal patches, and combinations thereof.

The AAT antisense agents of the invention may be formulated into an intravenous dosage form by any suitable method detailed in the techniques and composition references cited herein.

The AAT antisense agents of the invention may be formulated into an intranasal (transnasal) dosage form by any suitable method detailed in the techniques and composition references cited herein.

Tablets, capsules and intravenous formulations of presentation are provided in discrete units conveniently contain a daily dose, or an appropriate fraction thereof, of one or more of the AAT antisense agents of the invention. For example, the units may contain from about 1 mg to about 1000 mg, alternatively from about 5 mg to about 500 mg, alternatively from about 5 mg to about 250 mg, alternatively from about 10 mg to about 100 mg of one or more of the AAT antisense agents or combinations of the present invention.

Methods of Treatment:

The AAT antisense agents and pharmaceutical compositions of the present invention alone or in combination with other antibiotics can be administered to treat bacterial infections caused by Gram-negative and/or Gram-positive bacteria. More preferred, the AAT antisense agents and pharmaceutical compositions of the present invention alone or in combination with other antibiotics can be administered to treat bacterial infections caused by Gram-negative bacteria.

Typically, a therapeutically effective amount of the AAT antisense agents or pharmaceutical composition is administered to treat the infection.

The dose range for adult or paediatric human beings will depend on a number of factors including the age, weight and condition of the patient. Suitable oral dosages of the AAT antisense agents of the present invention can range from about 1 mg to about 2000 mg.

The AAT antisense agents or a combination of antibacterial agent(s) and AAT antisense agents may be administered once-a-day, or two, or three or more times a day. Preferably, the AAT antisense agents or combinations are administered by intravenous infusion from once-a-day to four times a day, preferably with each infusion lasting from 30 to 60 mins.

Polymorphs

The invention further relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

If different structural isomers are present, and/or one or more chiral centres are present, all isomeric forms are intended to be covered. Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985). It is also intended to include compounds of general formula (I) where any hydrogen atom has been replaced by a deuterium atom.

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase synthesis followed by purification. When referring to a sequence of the oligonucleotide, reference is made to the sequence or order of nucleobase moieties, or modifications thereof, of the covalently linked nucleotides or nucleosides starting at 5′-end and finishing at the 3′-end (irrespective of the backbone of the oligonucleotide). The oligonucleotide of the invention is man-made, and is chemically synthesized, and is typically purified or isolated. The oligonucleotide of the invention may comprise one or more modified nucleosides or nucleotides.

Antisense Oligonucleotides

The term “Antisense oligonucleotide” as used herein is defined as an oligonucleotide capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides employed in the compounds of the present invention are single stranded. It is understood that single stranded oligonucleotides employed in the compounds of the present invention can form hairpins or intermolecular duplex structures (duplex between two molecules of the same oligonucleotide), as long as the degree of intra or inter self-complementarity is less than 50% across of the full length of the oligonucleotide.

The term “antisense” refers to any composition containing a nucleic acid sequence which is complementary to the “sense” strand of a specific nucleic acid sequence. Once introduced into a cell, the complementary nucleotides combine with natural sequences produced by the cell to form duplexes and to block either transcription or translation, thereby altering gene expression and/or interfering with post-transcriptional RNA processing (e.g. splicing, microRNA regulation, etc.). The designation “negative” or “minus” can refer to the antisense strand, and the designation “positive” or “plus” can refer to the sense strand.

An “antisense oligomer”, “antisense oligonucleotide” or “ASO” refers to an antisense molecule or anti-gene agent that comprises an oligomer of at least about 10 nucleotides in length. In particular embodiments an antisense oligomer comprises at least 15, 18, 20, 25, 30, 35, 40, or 50 nucleotides. ASOs may be synthesized by standard methods known in the art. As examples, phosphorothioate oligomers may be synthesized by the method of Stein et al. (1988) Nucleic Acids Res. 16, 3209 3021), methylphosphonate oligomers can be prepared by use of controlled pore glass polymer supports (Sarin et al. (1988) Proc. Natl. Acad. Sci. USA. 85, 7448-7451). Morpholino oligomers may be synthesized by the method of Summerton and Weller U.S. Pat. Nos. 5,217,866 and 5,185,444.

The antisense oligomers included in the AAT ASOs of the present invention may target genes that contribute to virulence, antibiotic resistance, biofilm formation or essential growth and survival processes in bacteria. The antisense oligomers included in the AAT ASOs of the present invention may be useful in the monotherapy treatment of bacterial infections or, through the use of combinations with known antibiotics, useful in imparting improved and clinically meaningful activity (minimum inhibitory concentration; MIC) against bacterial infections.

Preferred Sub-Structures of Formula (I)

In an embodiment, p is 1.

In an embodiment, R₁ is selected from the group consisting of: H, C₁₋₆ alkyl, C₁₋₆ substituted alkyl, C₃₋₈ cycloalkyl and C₃₋₈ substituted cycloalkyl.

In an embodiment, R₁ is selected from the group consisting of: H, C₁₋₆ alkyl and C₁₋₆ substituted alkyl.

In an embodiment, R₁ is H.

In an embodiment, R₁ is C₁₋₆ alkyl. Preferably, R₁ is C₁₋₄ alkyl. More preferably, R₁ is Me.

In an embodiment, p is 0.

In an embodiment, n is 1.

In an embodiment, n is 2.

In an embodiment, R₂ and R₃ are each independently selected from the group consisting of: H, C₁₋₆ alkyl, C₁₋₆ substituted alkyl, C₃₋₈ cycloalkyl and C₃₋₈ substituted cycloalkyl.

In an embodiment, R₂ and R₃ are each independently selected from the group consisting of: H, C₁₋₆ alkyl and C₁₋₆ substituted alkyl.

In an embodiment, R₂ and R₃ are each independently selected from the group consisting of: H and C₁₋₆ alkyl.

In an embodiment, one of R₂ and R₃ is H and the other is C₁₋₆ alkyl. Preferably, one of R₂ and R₃ is H and the other is C₁₋₄ alkyl. More preferably, one of R₂ and R₃ is H and the other is Me.

In an embodiment, R₂ and R₃ are each H.

In an embodiment, R₄ and R₅ are each independently selected from the group consisting of: H, C₁₋₆ alkyl, C₁₋₆ substituted alkyl, C₃₋₈ cycloalkyl and C₃₋₈ substituted cycloalkyl.

In an embodiment, R₄ and R₅ are each independently selected from the group consisting of: H, C₁₋₆ alkyl and C₁₋₆ substituted alkyl.

In an embodiment, R₄ and R₅ are each independently selected from the group consisting of: H and C₁₋₆ alkyl.

In an embodiment, one of R₄ and R₅ is H and the other is C₁₋₆ alkyl. Preferably, one of R₄ and R₅ is H and the other is C₁₋₄ alkyl. More preferably, one of R₄ and R₅ is H and the other is Me.

In an embodiment, R₄ and R₅ are each H.

In an embodiment, m is 0 and R₄ and R₅ are absent. In an embodiment, m is 0 and R₄ and R₅ are absent and one of R₂ and R₃ is H and the other is C₁₋₆ alkyl. Preferably, m is 0 and R₄ and R₅ are absent and one of R₂ and R₃ is H and the other is C₁₋₄ alkyl. More preferably, m is 0 and R₄ and R₅ are absent and one of R₂ and R₃ is H and the other is Me. Most preferably, m is 0 and R₄ and R₅ are absent and the R₂ and R₃ groups are selected such that the moiety:

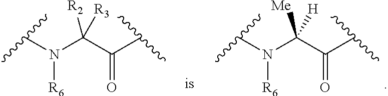

In an embodiment, m is 2 and R₄ and R₅ are H. In an embodiment, m is 0 and R₄ and R₅ are H and R₂ and R₃ are H.

In an embodiment, R₆ is selected from the group consisting of: H, C₁₋₆ alkyl, C₁₋₆ substituted alkyl, C₃₋₈ cycloalkyl and C₃₋₈ substituted cycloalkyl.

In an embodiment, R₆ is selected from the group consisting of: H, C₁₋₆ alkyl and C₁₋₆ substituted alkyl.

In an embodiment, R₆ is H.

In an embodiment, q is 1.

In an embodiment, q is 1 and L₂ has the structure:

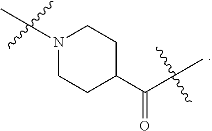 [SMILES: *C(=O)C1CCN(*)CC1]

In an embodiment, q is 1 and L₂ has the structure:

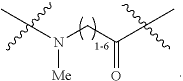

In an embodiment, q is 1 and L₂ has the structure:

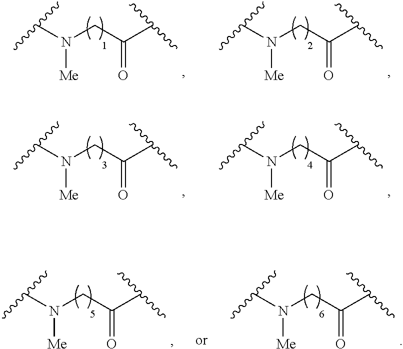

In an embodiment, q is 1 and L₂ has the structure:

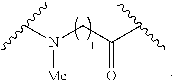

In an embodiment, q is 1 and L₂ has the structure:

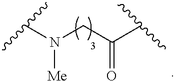

In an embodiment, q is 0.

Linker-1:

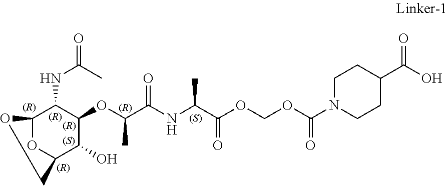

In an embodiment, p is 1, n is 1 and q is 1.

In an embodiment, p is 1, n is 1, q is 1, R₁ is H, one of R₂ and R₃ is H and the other is C₁₋₆ alkyl, m is 0 and R₄ and R₅ are absent, R₆ is H and L₂ has the structure:

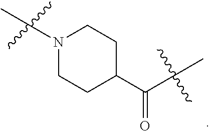 [SMILES: *C(=O)C1CCN(*)CC1]

In an embodiment, p is 1, n is 1, q is 1, R₁ is H, one of R₂ and R₃ is H and the other is Me, m is 0 and R₄ and R₅ are absent, R₆ is H and L₂ has the structure:

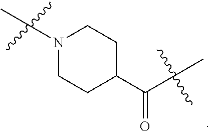 [SMILES: *C(=O)C1CCN(*)CC1]

In an embodiment, p is 1, n is 1, q is 1, R₁ is H, one of R₂ and R₃ is H and the other is Me, m is 0 and R₄ and R₅ are absent, R₆ is H, L₂ has the structure:

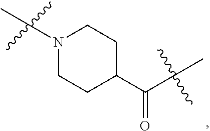 [SMILES: *C(=O)C1CCN(*)CC1]

and the R₂ and R₃ groups are selected such that the moiety:

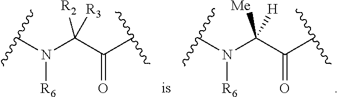

Linker-2:

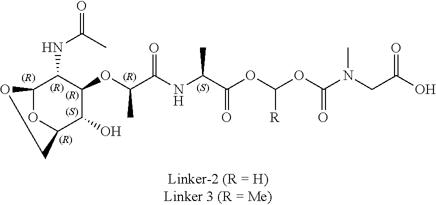

In an embodiment, p is 1, n is 1 and q is 1.

In an embodiment, p is 1, n is 1, q is 1, R₁ is H, one of R₂ and R₃ is H and the other is C₁₋₆ alkyl, m is 0 and R₄ and R₅ are absent, R₆ is H and L₂ has the structure:

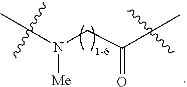

In an embodiment, p is 1, n is 1, q is 1, R₁ is H, one of R₂ and R₃ is H and the other is Me, m is 0 and R₄ and R₅ are absent, R₆ is H and L₂ has the structure:

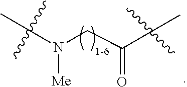

In an embodiment, p is 1, n is 1, q is 1, R₁ is H, one of R₂ and R₃ is H and the other is C₁₋₆ alkyl, m is 0 and R₄ and R₅ are absent, R₆ is H and L₂ has the structure:

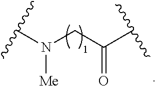

In an embodiment, p is 1, n is 1, q is 1, R₁ is H, one of R₂ and R₃ is H and the other is Me, m is 0 and R₄ and R₅ are absent, R₆ is H and L₂ has the structure:

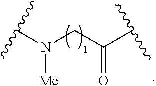

Linker-3:

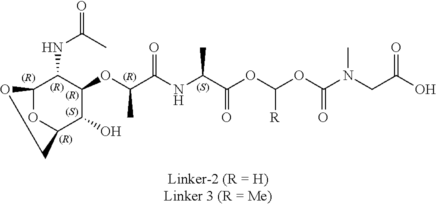

In an embodiment, p is 1, n is 1 and q is 1.

In an embodiment, p is 1, n is 1, q is 1, R₁ is C₁₋₆ alkyl, one of R₂ and R₃ is H and the other is C₁₋₆ alkyl, m is 0 and R₄ and R₅ are absent, R₆ is H and L₂ has the structure:

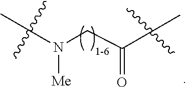

In an embodiment, p is 1, n is 1, q is 1, R₁ is C₁₋₆ alkyl, one of R₂ and R₃ is H and the other is Me, m is 0 and R₄ and R₅ are absent, R₆ is H and L₂ has the structure:

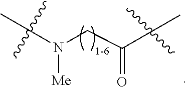

In an embodiment, p is 1, n is 1, q is 1, R₁ is Me, one of R₂ and R₃ is H and the other is C₁₋₆ alkyl, m is 0 and R₄ and R₅ are absent, R₆ is H and L₂ has the structure:

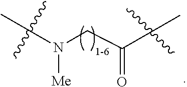

In an embodiment, p is 1, n is 1, q is 1, R₁ is Me, one of R₂ and R₃ is H and the other is Me, m is 0 and R₄ and R₅ are absent, R₆ is H and L₂ has the structure:

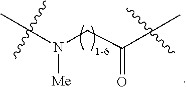

In an embodiment, p is 1, n is 1, q is 1, R₁ is C₁₋₆ alkyl, one of R₂ and R₃ is H and the other is C₁₋₆ alkyl, m is 0 and R₄ and R₅ are absent, R₆ is H and L₂ has the structure:

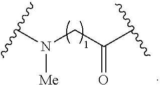

In an embodiment, p is 1, n is 1, q is 1, R₁ is Me, one of R₂ and R₃ is H and the other is Me, m is 0 and R₄ and R₅ are absent, R₆ is H and L₂ has the structure:

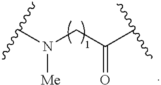

Linker-4:

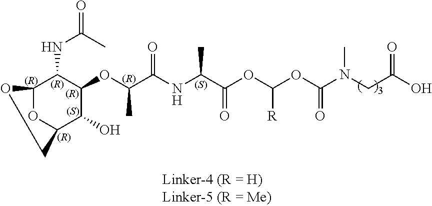

In an embodiment, p is 1, n is 1 and q is 1.

In an embodiment, p is 1, n is 1, q is 1, R₁ is H, one of R₂ and R₃ is H and the other is C₁₋₆ alkyl, m is 0 and R₄ and R₅ are absent, R₆ is H and L₂ has the structure:

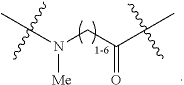

In an embodiment, p is 1, n is 1, q is 1, R₁ is H, one of R₂ and R₃ is H and the other is Me, m is 0 and R₄ and R₅ are absent, R₆ is H and L₂ has the structure:

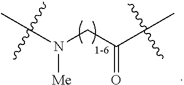

In an embodiment, p is 1, n is 1, q is 1, R₁ is H, one of R₂ and R₃ is H and the other is C₁₋₆ alkyl, m is 0 and R₄ and R₅ are absent, R₆ is H and L₂ has the structure:

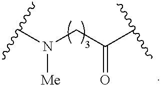

In an embodiment, p is 1, n is 1, q is 1, R₁ is H, one of R₂ and R₃ is H and the other is Me, m is 0 and R₄ and R₅ are absent, R₆ is H and L₂ has the structure:

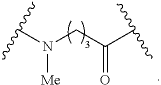

Linker-5:

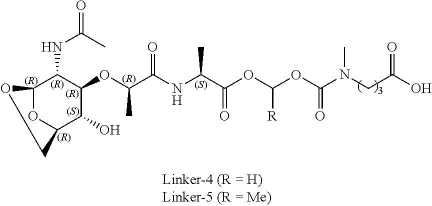

In an embodiment, p is 1, n is 1 and q is 1.

In an embodiment, p is 1, n is 1, q is 1, R₁ is C₁₋₆ alkyl, one of R₂ and R₃ is H and the other is C₁₋₆ alkyl, m is 0 and R₄ and R₅ are absent, R₆ is H and L₂ has the structure:

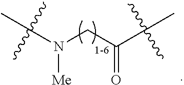

In an embodiment, p is 1, n is 1, q is 1, R₁ is C₁₋₆ alkyl, one of R₂ and R₃ is H and the other is Me, m is 0 and R₄ and R₅ are absent, R₆ is H and L₂ has the structure:

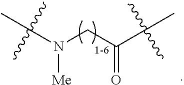

In an embodiment, p is 1, n is 1, q is 1, R₁ is Me, one of R₂ and R₃ is H and the other is C₁₋₆ alkyl, m is 0 and R₄ and R₅ are absent, R₆ is H and L₂ has the structure:

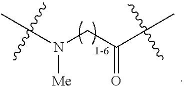

In an embodiment, p is 1, n is 1, q is 1, R₁ is Me, one of R₂ and R₃ is H and the other is Me, m is 0 and R₄ and R₅ are absent, R₆ is H and L₂ has the structure:

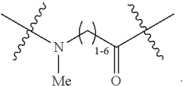

In an embodiment, p is 1, n is 1, q is 1, R₁ is C₁₋₆ alkyl, one of R₂ and R₃ is H and the other is C₁₋₆ alkyl, m is 0 and R₄ and R₅ are absent, R₆ is H and L₂ has the structure:

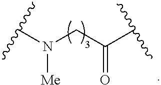

In an embodiment, p is 1, n is 1, q is 1, R₁ is Me, one of R₂ and R₃ is H and the other is Me, m is 0 and R₄ and R₅ are absent, R₆ is H and L₂ has the structure:

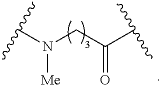

Linker-6:

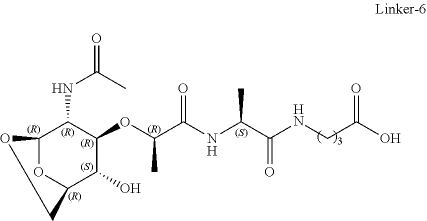

Linker-6

In an embodiment, p is 0, n is 2 and q is 0, wherein one

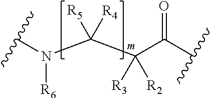

residue is

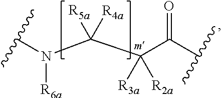

wherein R₂ₐ, R₃ₐ, R₄ₐ, R₅ₐ, R₆ₐ and m′ have the same respective definition as the moieties R₂, R₃, R₄, R₅, R₆ and m (as described in any embodiment herein), but may be independently selected therefrom.

In an embodiment, p is 0, n is 2, q is 0, R₂ and R₃ are each independently selected from the group consisting of: H and C₁₋₆ alkyl, R₂ₐ and R₃ₐ are each H, R₄, R₅, R₄ₐ and R₅ₐ are each H, m is 0, m′ is 2, R₆ is H and R₆ₐ is H.

In an embodiment, p is 0, n is 2, q is 0, R₂ and R₃ are each independently selected from the group consisting of: H and Me, R₂ₐ and R₃ₐ are each H, R₄, R₅, R₄ₐ and R₅ₐ are each H, m is 0, m′ is 2, R₆ is H and R₆ₐ is H.

In an embodiment,

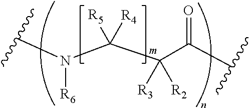

is bonded to the sugar moiety on the left side and

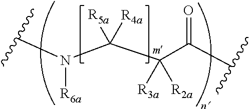

is bonded to the antisense moiety on the right side.

In an embodiment, R₁ is selected from the group consisting of: H, C₁₋₆ alkyl, C₁₋₆ substituted alkyl, C₃₋₈ cycloalkyl and C₃₋₈ substituted cycloalkyl; R₂ and R₃ are each independently selected from the group consisting of: H, C₁₋₆ alkyl, C₁₋₆ substituted alkyl, C₃₋₈ cycloalkyl and C₃₋₈ substituted cycloalkyl; R₄ and R₅ are each independently selected from the group consisting of: H, C₁₋₆ alkyl, C₁₋₆ substituted alkyl, C₃₋₈ cycloalkyl and C₃₋₈ substituted cycloalkyl; and R₆ is selected from the group consisting of: H, C₁₋₆ alkyl, C₁₋₆ substituted alkyl, C₃₋₈ cycloalkyl and C₃₋₈ substituted cycloalkyl.

In an embodiment, R₁ is selected from the group consisting of: H, C₁₋₆ alkyl and C₁₋₆ substituted alkyl; R₂ and R₃ are each independently selected from the group consisting of: H, C₁₋₆ alkyl and C₁₋₆ substituted alkyl; R₄ and R₅ are each independently selected from the group consisting of: H, C₁₋₆ alkyl and C₁₋₆ substituted alkyl; and R₆ is selected from the group consisting of: H, C₁₋₆ alkyl and C₁₋₆ substituted alkyl.

In an embodiment, R₁ is H; R₂ and R₃ are each independently selected from the group consisting of: H and C₁₋₆ alkyl; R₄ and R₅ are each independently selected from the group consisting of: H and C₁₋₆ alkyl; and R₆ is H.

In an embodiment, R₁ is H; one of R₂ and R₃ is H and the other is C₁₋₆ alkyl, preferably, one of R₂ and R₃ is H and the other is C₁₋₄ alkyl; one of R₄ and R₅ is H and the other is C₁₋₆ alkyl, preferably, one of R₄ and R₅ is H and the other is C₁₋₄ alkyl; and R₅ is H.

In an embodiment, R₁ is selected from the group consisting of: H, C₁₋₆ alkyl, C₁₋₆ substituted alkyl, C₃₋₈ cycloalkyl and C₃₋₈ substituted cycloalkyl; R₂ and R₃ are each independently selected from the group consisting of: H, C₁₋₆ alkyl, C₁₋₆ substituted alkyl, C₃₋₈ cycloalkyl and C₃₋₈ substituted cycloalkyl; m is 0; and R₆ is selected from the group consisting of: H, C₁₋₆ alkyl, C₁₋₆ substituted alkyl, C₃₋₈ cycloalkyl and C₃₋₈ substituted cycloalkyl.

In an embodiment, R₁ is selected from the group consisting of: H, C₁₋₆ alkyl and C₁₋₆ substituted alkyl; R₂ and R₃ are each independently selected from the group consisting of: H, C₁₋₆ alkyl and C₁₋₆ substituted alkyl; m is 0; and R₆ is selected from the group consisting of: H, C₁₋₆ alkyl and C₁₋₆ substituted alkyl.

In an embodiment, R₁ is H; R₂ and R₃ are each independently selected from the group consisting of: H and C₁₋₆ alkyl; m is 0; and R₆ is H.

In an embodiment, R₁ is H; one of R₂ and R₃ is H and the other is C₁₋₆ alkyl, preferably, one of R₂ and R₃ is H and the other is C₁₋₄ alkyl; m is 0; and R₆ is H.

In an embodiment, R₁ is C₁₋₆ alkyl; R₂ and R₃ are each independently selected from the group consisting of: H and C₁₋₆ alkyl; R₄ and R₅ are each independently selected from the group consisting of: H and C₁₋₆ alkyl; and R₆ is H.

In an embodiment, R₁ is C₁₋₆ alkyl, preferably C₁₋₄ alkyl; one of R₂ and R₃ is H and the other is C₁₋₆ alkyl, preferably, one of R₂ and R₃ is H and the other is C₁₋₄ alkyl; one of R₄ and R₅ is H and the other is C₁₋₆ alkyl, preferably, one of R₄ and R₅ is H and the other is C₁₋₄ alkyl; and R₆ is H.

In an embodiment, R₁ is C₁₋₆ alkyl; R₂ and R₃ are each independently selected from the group consisting of: H and C₁₋₆ alkyl; m is 0; and R₆ is H.

In an embodiment, R₁ is C₁₋₆ alkyl, preferably C₁₋₄ alkyl; one of R₂ and R₃ is H and the other is C₁₋₆ alkyl, preferably, one of R₂ and R₃ is H and the other is C₁₋₄ alkyl; m is 0; and R₆ is H.

In an embodiment, p is 0 and n is 0.

In an embodiment p is 1, n is 1, 2, 3 or 4 and q is 1. In an embodiment p is 1, n is 1 and q is 1.

In an embodiment p is 1, n is 0 and q is 1.

In an embodiment, p is 0, n is 0 and q is 1.

In an embodiment, R₇ is H. In an embodiment, R₇ is acetyl. In an embodiment, R₇ is benzoyl.

In an embodiment, R₈ is H. In an embodiment, R₈ is acetyl. In an embodiment, R₈ is benzoyl.

In an embodiment, R₉ is H. In an embodiment, R₉ is acetyl. In an embodiment, R₉ is benzoyl.

In an embodiment, R₁₀ is methyl. In an embodiment, R₁₀ is ethyl. In an embodiment, R₁₀ is propyl.

By way of example, compounds according to the present invention include but are not limited from the following preferred substructures:

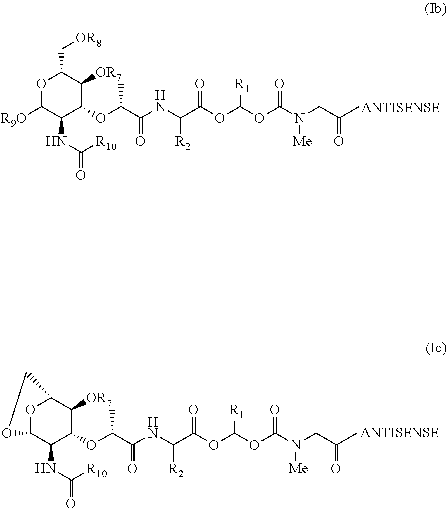

wherein m is selected as ‘0’, n is selected as ‘1’, p is selected as ‘1’, q is selected as ‘1, R₃ is H, R₆ is H and “SPACER” is N-methylglycine and R₁, R₂, R₇, R₈, R₉ and R₁₀ are as defined above; or

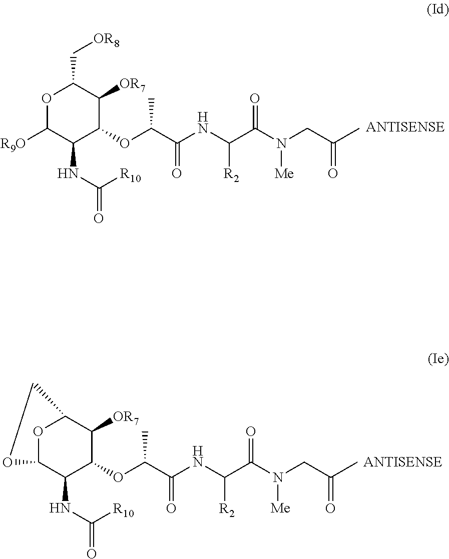

wherein m is selected as ‘0’, n is selected as ‘1’, p is selected as ‘0’, q is selected as ‘1’, R₃ is H, R₆ is H and “SPACER” is N-methylglycine and R₂, R₇, R₈, R₉ and R₁₀ are as defined above; or

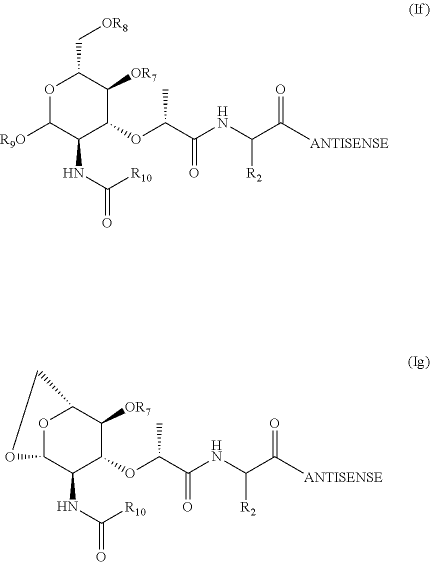

wherein m is selected as ‘0’, n is selected as ‘1’, p is selected as ‘0’, q is selected as ‘0’, R₃ is H, and R₂, R₇, R₈, R₉ and R₁₀ are as defined above; or

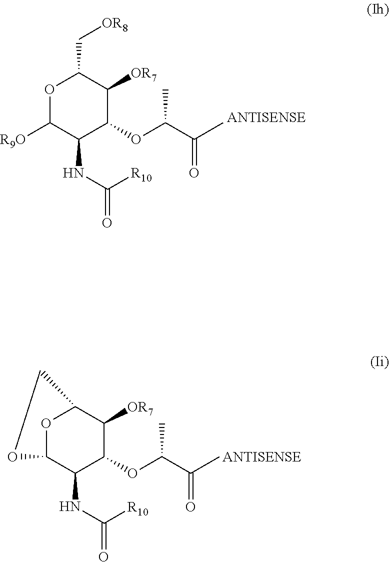

wherein n is selected as ‘0’, p is selected as ‘0’, q is selected as ‘0’, and R₇, R₈, R₉ and R₁₀ are as defined above;

In a preferred embodiment substructures (Ic), (Ie), (Ig), and (Ii) are chosen.

In an even more preferred embodiment substructures (Ic) and (Ig) are chosen.

By way of example, compounds according to the present invention include but are not limited from the following combination of features:
n is 1, m is 0, R₆ is H, R₂ is H, R₃ is Me, p is 1, R₁ is H or Me, and L₂ has the structure:

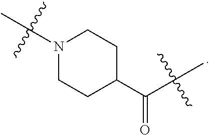 [SMILES: *C(=O)C1CCN(*)CC1]

n is 1, m is 0, R₆ is H, R₂ is H, R₃ is Me, p is 1, R₁ is H or Me, and L₂ has the structure:

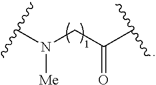

n is 1, m is 0, R₆ is H, R₂ is H, R₃ is Me, p is 1, R₁ is H or Me, and L₂ has the structure:

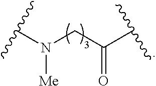

n is 1, m is 0, R₆ is H, R₂ is H, R₃ is Me, p is 0 and L₂ has the structure:

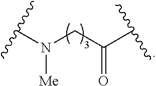

Preferred Antisense Agents

The ANTISENSE agent contains a terminal amino functional group for chemical bond formation with the remainder of the molecule to provide molecules of general formula (I). The following Tables 1A, 1B, 1C, 1D are not intended to be an exhaustive list of potential antisense targets but detail the sequences of bases that the ANTISENSE agent might include.

Preferred ASOs are of sufficient length and complementarity to specifically hybridize to a bacterial nucleic acid target that encodes a protein in a biochemical pathway and/or cellular process that is essential for bacterial survival and growth. General examples of such biochemical pathways or cellular processes include cell division, murein biosynthesis, global regulatory mechanisms, fatty acid biosynthesis, DNA replication, ribosomal proteins, transcription, translation initiation, lipopolysaccharide biosynthesis, nucleic acid biosynthesis, biofilm growth and intermediary metabolism. Particular examples of genes in biochemical pathways and cellular processes include: RpsJ and RpmB (ribosomal proteins); LpxC, WaaC, WaaG, WaaA, WaaF, LpxA, LpxB (lipopolysaccharide biosynthesis); murA, mraY, murB, murC, murE, murF, murG (murein peptidoglycan biosynthesis); acpP, accA, accB, fabG, fabZ (fatty acid biosynthesis); acpS (acyl carrier protein synthase); fabI (enoyl-acyl carrier protein reductase); fabD (malonyl coenzyme A acyl carrier protein transcyclase); folP (dihydropteroate synthase); fmhB (protein in glycine attachement); gyrA (DNA gyrase subunit); adk (adenylate kinase, cell energy homeostasis); infA (protein biosynthesis); ftsZ (cell division); rpoD (RNA synthesis); aroC (aromatic compound biosynthesis); inhA (enoyl-acyl carrier protein reductase); ompA (outer membrane protein A); blaT, cml, adeA (antibiotic resistance-associated genes); cepL, cepR, suhB, CsuE, SecA, Pg1L, PiIU1, AlgZ, AlgU, LasR, FleR, PelF (biofilm formation-associated).

TABLE 1A
Exemplary Antibiotic Resistance Targeting
Sequences

| Target | Antisense | Sequence |
|---|---|---|
| Gene | sequence (5′-3′) | ID |
| NDM-1 | TCA AGT TTT CC | 1 |
| NDM-1 | TCC TTT TAT TC | 2 |
| NDM-1 | CCA TCA AGT TT | 3 |
| NDM-1 | GGC AAT TCC AT | 4 |
| adeA | ATA CTG TCC AA | 5 |
| OmpA | CAT GGA TAT CC | 6 |
| AcrA | ATG TAA ACC TC | 7 |
| AcrA | GTT CAT ATG TA | 8 |
| AcrA | ACC CCT CTG TT | 9 |
| AcrA | TGT TCA TAT GT | 10 |
| AcrB | GTC TTA ACG GC | 11 |
| AcrB | AGG CAT GTC TT | 12 |
| AcrB | TAG GCA TGT CT | 13 |
| AcrR | TAT GTT CGT GA | 14 |
| TolC | TTC ATT TGC AT | 15 |
| TolC | ATT CCT TGT GG | 16 |
| TolC | TTT GCA TTC CT | 17 |
| KPC | GAT ACA GTG AC | 18 |
| KPC 1-4 | AAC GAT ATT CC | 19 |

TABLE 1B
Exemplary Biofilm Formation Targeting Sequences

| Target | Antisense | Sequence |
|---|---|---|
| Gene | sequence (5′-3′) | ID |
| cepl | AAG GTC TGC AT | 20 |
| cepl | TCG GAT CTG TG | 21 |
| cepl | CAT GGA TGT CC | 22 |
| cepl | CGT GAA CGA AG | 23 |
| cepl | CGT GTG GCA AC | 24 |
| cepl | GCC CGA GAT CC | 25 |
| cepl | CTT TCG TTC GC | 26 |
| suhB | ATG CAT GAG CC | 27 |
| suhB | GGA TGC ATG AG | 28 |
| CsuE | TTA TAT TCA TGG | 29 |
| CsuE | TCA TGG CAA AG | 30 |
| CsuE | TTT CCT GTC AA | 31 |
| SecA | TTG CCA ACA TG | 32 |
| Pg1L | CAT TAC CCA AG | 33 |
| PilU1 | TTA AAA TCC AT | 34 |
| AlgZ | TAG GCA TCG AC | 35 |
| AlgU | AAA GCT CCT CT | 36 |
| LasR | AGG CCA TAG CG | 37 |
| FleR | TTA CTC CTG AA | 38 |
| PelF | TTC GGT CAT GT | 39 |

TABLE 1C
Exemplary Fatty Acid Biosynthesis-associated
Targeting Sequences

| Target | Antisense | Sequence |
|---|---|---|
| Gene | sequence (5′-3′) | ID |
| acpP | GTC CAT TAC CC | 40 |
| acpP | CAT TAC CCC TC | 41 |
| acpP | CCA TTA CCC CT | 42 |
| acpP | TCC ATT ACC CC | 43 |
| acpP | TGT CCA TTA CC | 44 |
| acpP | TTG TCC ATT AC | 45 |
| acpP | GTT GTC CAT TA | 46 |
| acpP | TGT TGT CCA TT | 47 |
| acpP | ATG TTG TCC AT | 48 |
| acpP | TTT ACA AGT GC | 49 |
| acpP | CCT CCG AGG GA | 50 |
| acpP | ACA CGT TGT TC | 51 |
| acpP | AGT TCA GCG AC | 52 |
| acpP | CTC ATA CCT TG | 53 |
| acpP | TGC TCA TAC TC | 54 |
| acpP | CTC ATA CTC T | 55 |
| acpP | CTC ATA CTA T | 56 |
| acpP | CTT CGA TAG TG | 57 |
| acpP | ATA TCG CTC AC | 58 |
| acpP | ATT CTC CTC AT | 59 |
| acpP | CAC AGG AAT TC | 60 |
| acpP | CAT TGC TTG TG | 61 |
| acpP | CAT ACC TTG TT | 62 |
| acpS | TTG CCA TTA GC | 63 |
| acp-E | CTG TAG TGA TTT CAC CA | 64 |
| fabA | TTA TCT ACC AT | 65 |
| fabB | CGT TTC ATT AA | 66 |
| fabB | GCA CGT TTC AT | 67 |
| fabI | AGA AAA CCC AT | 68 |
| fabI | GCT TTA ATC C | 69 |
| fabI | CCC ATA GCT T | 70 |
| fabI | CAT GTA AGA T | 71 |
| fabI | AGA TAA CTC C | 72 |
| gapA | TTG ATA GTC AT | 73 |
| accA | GCT TTT TTC AT | 74 |
| accA | AGG CTT CCG TC | 75 |
| fabD | GTC ATG TTT T | 76 |
| inhA | GTC ATT TGG T | 77 |
| inhA | CAT TTG GTG ACT | 78 |

TABLE 1D
Exemplary Targeting Sequences associated with
other pathways or cellular processes

| Target | Antisense | Sequence |
|---|---|---|
| Gene | sequence (5′-3′) | ID |
| RpoD | TCA TCT TTG CT | 79 |
| RpoD | TTT TGC TCC AT | 80 |
| PilB | AGT AAC TCC AC | 81 |
| murA | TTT ATC CAT TG | 82 |
| rpsJ | GCA TTT GAC CT | 83 |
| rpsJ | TAG ACA TAC CA | 84 |
| rpsJ | TAG CAG TAA AC | 85 |
| rpsJ | TGG TTC TGC AT | 86 |
| rpsJ | CCT CAG ACT CC | 87 |
| rpsJ | GCA TTT GAC CT | 88 |
| ftsZ | AGT TTC TCT CC | 89 |
| ftsZ | GTT CAA ACA TA | 90 |
| ftsZ | TCA AAT GAG GC | 91 |
| ftsz | AAT GAG GCC AT | 92 |
| ftsZ | ATA GTT TCT CTC C | 93 |
| gyrA | CGC TCA TCT AA | 94 |
| gyrA | CTA TAC ATA GAC | 95 |
| gyrA | GCC ATC TCG GAC ATC | 96 |
| gyrA | ATA CCA GGT GTT ATC T | 97 |
| dnaB | TTC CTG CCA TA | 98 |
| LpxC | TTT GAT CAT CG | 99 |
| LpxC | TGT TTG ATC AT | 100 |
| LpxC | TGT TTC ACC AT | 101 |
| LpxC | GTT GTT TGA TC | 102 |
| 23S rRNA | AGT GCT CTA CC | 103 |
| 23S rRNA | GCC TGT TAT CC | 104 |
| 16S rRNA | CCA TGC AGC AC | 105 |
| 16S rRNA | TTG CGC TCG TT | 106 |
| 16S rRNA | GGC TGC TGG CA | 107 |
| fmhB | CCA TGA AAA A | 108 |
| polA | TTC ATG CCT GT | 109 |
| murA | ATC CAT TTA GT | 110 |
| murA | CAT TTA GTT TG | 111 |
| murA | AAT TTA TCC AT | 112 |
| murA | AAA TTT ATC CA | 113 |
| rpmB | ACT CGG GAC AT | 114 |
| rpmB | CTA TTC TCC AA | 115 |
| rpmB | GGC AGA CTC GG | 116 |
| rpmB | CTT AGA CAT GG | 117 |
| adk | ATG ATA CGC AT | 118 |
| adk | AGT GCC CTC C | 119 |
| infA | TCT TTG GCC AT | 120 |

TABLE 1D
Exemplary Targeting Sequences associated with
other pathways or cellular processes

| Target | Antisense | Sequence |
|---|---|---|
| Gene | sequence (5′-3′) | ID |
| aroC | TTT CCA GCC AT | 121 |
| aroC | TTC CCT GCC AT | 122 |
| murF | ACG CTA ATC AT | 123 |
| murF | ACC TCC CAG GC | 124 |
| kdtA | AAT TCG AGC AT | 125 |
| boxA | TGT TTA AGA GC | 126 |
| boxA | CTC TTA ATG AT | 127 |
| boxC | ATC CAC CAC AG | 128 |
| rpoD-E | CTT GTA ACC ACA CCA | 129 |
| rpoD-E | TCC ACC AAG TCA CCA | 130 |
| pryC | GGT GCA GTC AT | 131 |
| pryC | AGA GTT CAA GG | 132 |
| pryA | GAC TTA ATC AA | 133 |
| lgt | CTA CTG GTC AT | 134 |
| folA | CAT TGA GAT TT | 135 |
| infB | ACA TCT GTC AT | 136 |
| nrdA | TTC TGA TTC AT | 137 |
| nrdB | GTA TAT GCC AT | 138 |
| zipA | TCC TGC ATC AT | 139 |
| caoA | ATA TAC CTC AT | 140 |
| gyrA-E | GTT ACC CTG ACC GAC CA | 141 |
| gyrA-E | GTT ACC CTG ACC ACC A | 142 |
| mrdA | TGT TTC ATA CG | 143 |
| LpxB | GGT TTG CCA AG | 144 |
| LpxB | TAA TCC GTC AG | 145 |
| carA | GGT GCT CAA AC | 146 |
| adeA | ATA CTG TAA AA | 147 |
| blaT | CTC TTC CTT TT | 148 |
| crmI | TCC TTC TGA TT | 149 |
| folP | ATG TTA TCC C | 150 |
| FmhB | CCA TGA TTT A | 151 |
| hmrB | TCC ACG TCG A | 152 |
| rpmB | GTC TAT TCT CC | 153 |
| rpmB | GAG ATG TCT AT | 154 |
| hmrB | TCC ACG TCG A | 155 |
| FabG | TTC TCT CCT TT | 156 |
| RpmB | CTC TAG ACA TG | 157 |
| WaaC | AGC ACC CTC AT | 158 |
| MryA | TGA CTC TCC TC | 159 |
| MurC | CCA CCT CCA GG | 160 |
| LpxA | ATC AAA CTC AT | 161 |
| WaaG | GCC AGG GTC AT | 162 |
| WaaA | GTACGG TTC AT | 163 |
| murB | CAG TCG CCC CT | 164 |
| murE | AGG CTC ATA GG | 165 |
| AccB | CTA GCA CTC CC | 166 |
| FabZ | ATG TCC ATC AT | 167 |
| MurG | GCA AAG TCC TC | 168 |
| AmpR | GTC GAA CCA AT | 169 |
| LepB | ATT GAG TGT CAT | 170 |
| LptD | TGC CAT CTT GTT | 171 |
| MraY | CAG GAG CAT TAG | 172 |

One skilled in the art will appreciate that the sequences listed in Tables 1A, 1B, 1C and 1D describe targeting antisense sequences and these may be increased in length through the addition of extra monomer units to either or both the 5′- and 3′-ends. Also, the targeting sequences listed in Tables 1A, 1B, 1C and 1D may differ by one, two or three monomer units and still retain the ability to bind to the bacterial nucleic acid of interest.

Preferred “Sugar Reagents”

The “SUGAR” of the present invention is prepared through the utilisation of the “Sugar Reagents”. Utilisation of the “Sugar Reagents” provides chemoselective formation of the chemical bond (primarily an amide bond formation) between the α-carbonyl of the terminal carboxylic acid of the “Sugar Reagent” and the remainder of the molecule of general formula I. When the terminal carboxylic acid of the “Sugar Reagent” is the α-carbonyl of the lactyl residue of the “SUGAR”, the following are known and preferred reagents (2-6) for these steps;

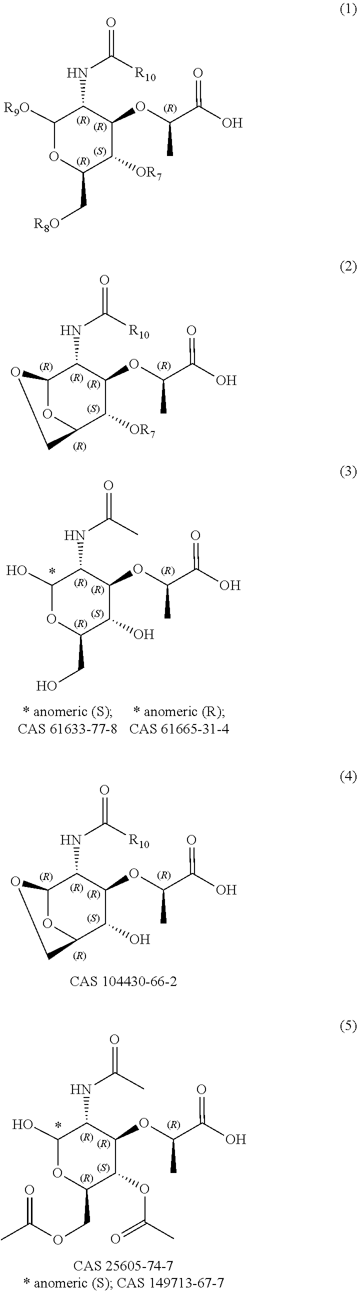

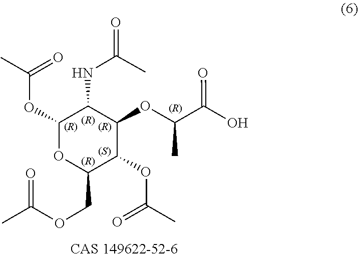

These reagents are commercially available or full synthetic procedures have been described in the literature (e.g. for (3), commercially available and see Merten, H and Brossmer, R., Carbohydrate Res., 191(1), 144-9, 1991; for (4) see Paulsen, H et al., Liebigs Annalen der Chemie, 4, 664-74, 1986; Hesek, 5 D. et al., JACS, 131(14), 5187-93, 2009; Wang, Q. et al., Org. Biomol. Chem. 14(3), 1013-23, 2016; Calvert, M. B. et al., Beilstein J. Org. Chem. 13, 2631-2636, 2017 for (5) see Osawa, T et al., Biochemistry, 8(8), 3369-75, 1969; for (6) see Wacker, O and Traxler, P. EP541486.

In the variation wherein ‘n’ is chosen as 1, wherein for example the terminal carboxylic acid of the “Sugar Reagent” is the α-carbonyl of an L-Alanine residue, it may be advantageous to extend reagents (2-6) and use these further intermediates in subsequent reactions. In this tactical variation, the following are preferred reagents (7 to 9b) for these steps;

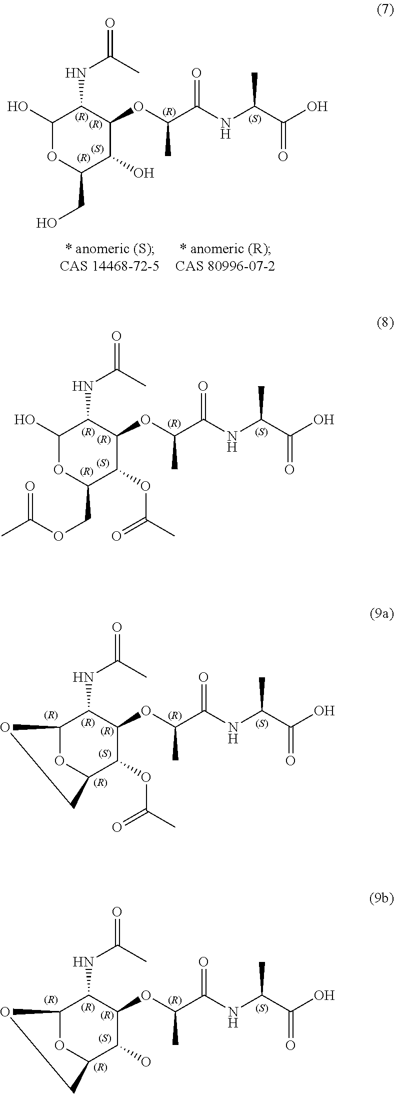

These reagents have full synthetic procedures described in the literature or are available by simple adaptations (e.g. for (7) see Chaturvedi, N. C. at al, J. Med. Chem., 9, 971-3, 1966; Klaic, B. Carbohydrate Res., 110(2), 320-5, 1982; for (8) see Bacic, A. and Pecar, S. Tet. Assym., 19, 2265-71, 2008; for (9b) see WO2014/002039, cpd 17 pg 87 wherein (S)-amphetamine can simply be replaced by L-alanine benzyl ester and synthesis commences from commercially available CAS 55682-47-8 (2S, 3R, 4R)-4-azido-2-(benzyloxy)-6,8-dioxabicyclo[3.2.1]octan-3-ol). Also see WO2016/172615 wherein routes to N-acyl variants of the N-acetyl reagent (7-9b) are detailed.

In a further variation wherein ‘n’ is chosen as 1, it may be advantageous to prepare a LINKER-SPACER-ANTISENSE intermediate and then utilise reagents such as (1-4).

In a further variation wherein ‘n’ is chosen as 1, it may be advantageous to prepare a SPACER-ANTISENSE intermediate and then utilise reagents such as (7-9b).

In yet a further variation, it may be advantageous to prepare an ANTISENSE intermediate and then utilise reagents such as (1-4, 7-9b or 9c-f).

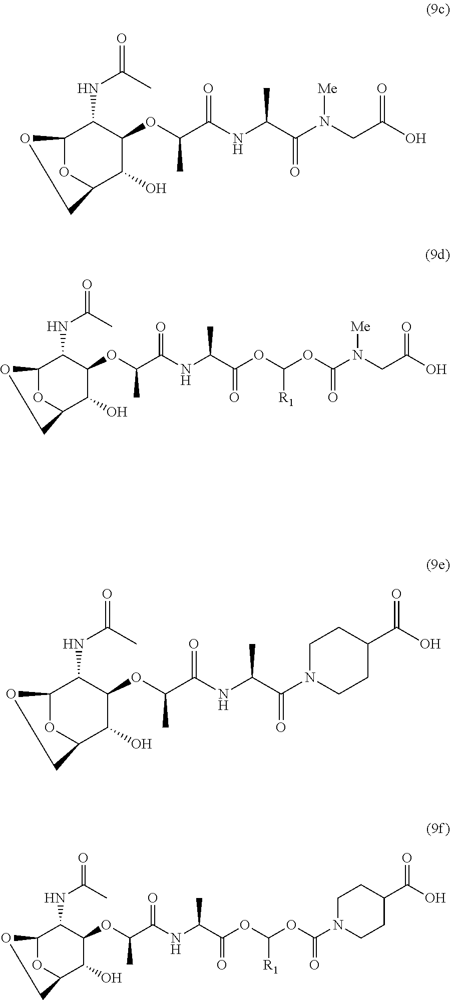

In additional variations wherein ‘n’ is chosen as 2, 3 or 4 it may be advantageous to extend “Sugar Reagents” (7-9b) and use these further intermediates that contain increasing similarity to the full structure of the bacterial cell wall peptide (NAc-Mur-L-Ala-D-Glu-meso-DAP-D-Ala) in subsequent reactions. In this tactical variation, the following are preferred reagents (10 to 18) for these steps. Reagents 10 to 18 may be prepared from the reagents such as 1 to 9 by standard peptide synthesis methods well known to those in the art. In order to provide chemoselective reaction with the terminal α-carboxylic acid of the “Sugar Reagents” 10 to 18, the side-chain carboxylic acid functional group of D-Glutamic acid and where present the side-chain carboxylic acid functional group and the sidechain amino functional group of meso-diaminopimelic acid (DAP) are protected with a protecting group Pg. Preferred protecting groups are the benzyl or tert-butyl ester and the benzyloxycarbonyl (Cbz) and tert-butoxycarbonyl (Boc) urethanes.

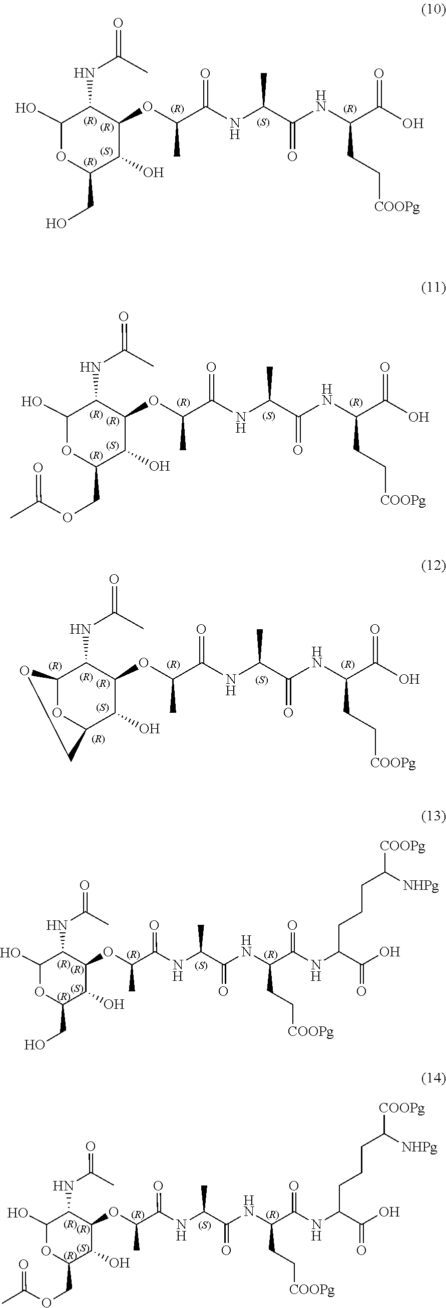

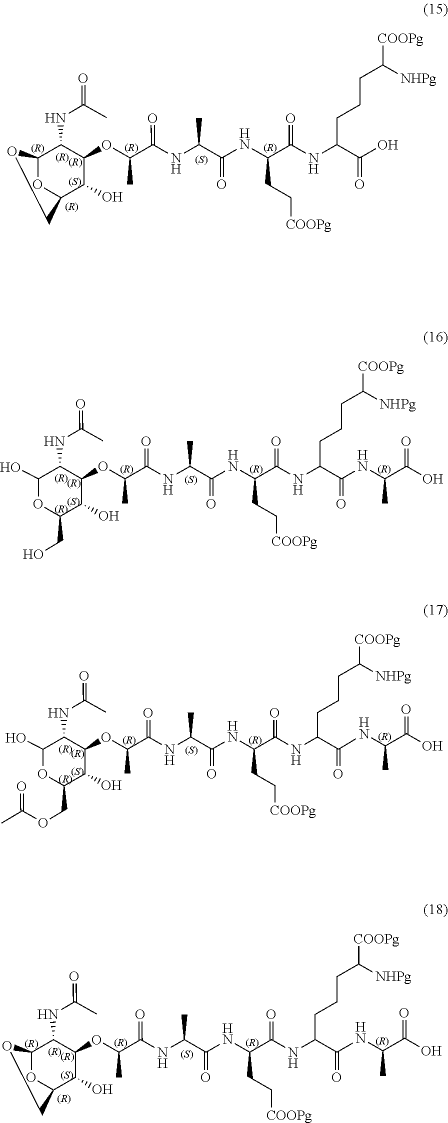

### Preparation of the ‘SUGAR-LINKER-SPACER-ANTISENSE AGENT’ of the Invention

Compounds of the present invention are prepared by the general methods provided herein and detailed in Schemes 3 and 4. One skilled in the art will recognize that the methods are by no means an exhaustive description and other routes may be available to make similar compounds. One skilled in the art will also appreciate that the vast majority of ANTISENSE agents contain more than one functional group that may participate in chemical reactions. Therefore, within the schemes “ANTISENSE” refers to intermediates wherein the nucleobases are temporarily protected during synthesis. As a final step in all schemes, the selective removal of the protecting group(s) “PGs” provides the compounds of general formula (I). For a description of an example synthesis of PNAs see Lee, H. et al., Org. Lett. 9(17), 3291-3, 2007. For a description of an example synthesis of PMOs see Summerton, J. and Weller, D. Antisense & Nucl. Acid Drug Dev., 7, 187-195, 1997. For a description of an example coupling to PMOs see O'Donovan, L. et al., Nucl. Acid Ther., 25(1), 1-10, 2015.

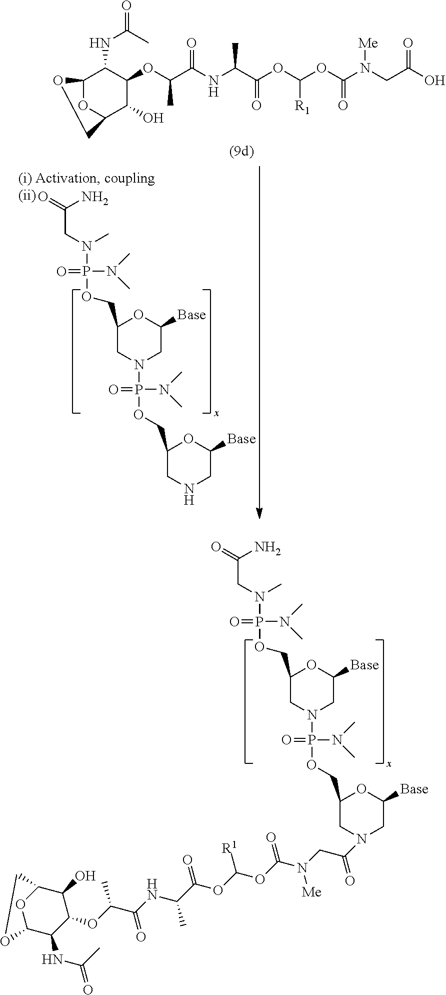

Scheme 3. General synthesis of compounds of formula (I) wherein “ANTISENSE” is a PMO conjugated at the 3′-end by a SUGAR-LINKER-SPACER. In an analogous manner, sugar reagent (9d) can readily be replaced by sugar reagents such as 1 to 18 to provide variants covering the range of definitions within formula (I).

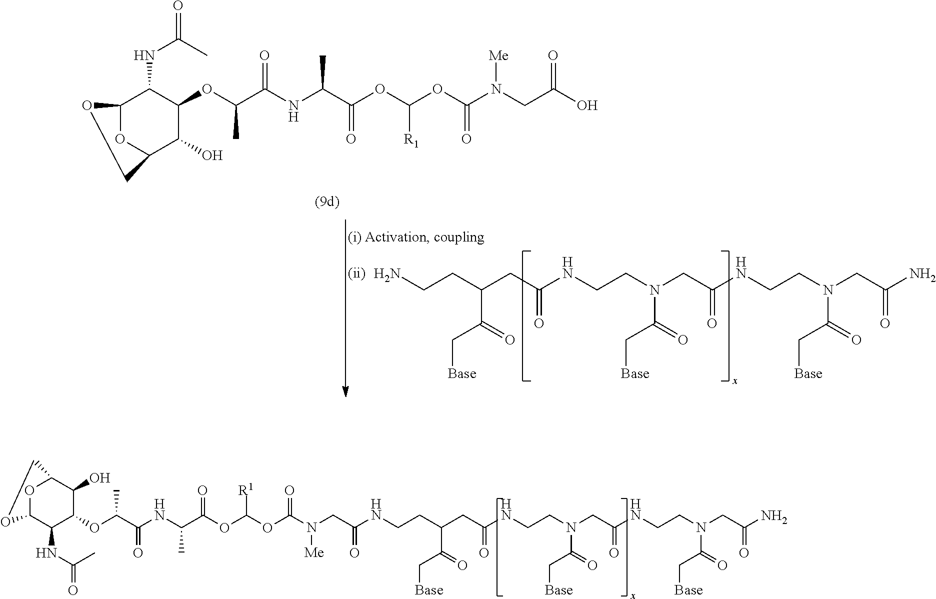

Scheme 4. General synthesis of compounds of formula (I) wherein “ANTISENSE” is a PNA conjugated at the N-terminus by a SUGAR-LINKER-SPACER. In an analogous manner, sugar reagent (9d) can readily be replaced by sugar reagents such as 1 to 18 to provide variants covering the range of definitions within formula (I).

For variants of the R₁ group, a range of chloroalkoxyacylchlorides are known or commercially available, e.g.,

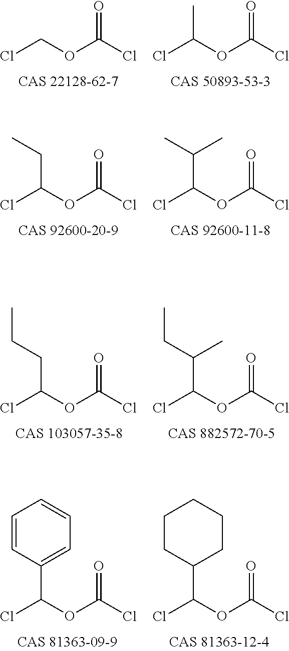

### EXAMPLES

The present invention is further illustrated by reference to the following Examples. However, it should be noted that these Examples, like the embodiments described above, are illustrative and are not to be construed as restricting the enabled scope of the invention in any way.

The following examples serve to more fully describe the manner of making and using the above-described invention. It is understood that these examples in no way serve to limit the true scope of the invention, but rather are presented for illustrative purposes.

Synthetic Chemistry

In the examples and the synthetic schemes below, the following abbreviations have the following meanings. If an abbreviation is not defined, it has its generally accepted meaning.
AcOH acetic acid
Ac₂O acetic anhydride
aq aqueous
BOC (Boc)N-tert-butoxycarbonyl or tert-butyloxycarbonyl
CBz carboxybenzyl
DCE dichloroethane
DCM dichloromethane
DCM/EA dichloromethane/ethanol
DIPEA (or DIEA) N,N-diisopropylethylamine, or Hünig's base
DME dimethoxyethane
DMF dimethylformamide
DMP Dess-Martin periodinane
DMSO-d6 deuterated dimethylsulfoxide
DMSO dimethylsulfoxide
EC₅₀ 50% effective concentration
EDTA ethylenediaminetetraacetic acid
Et ethyl
Et₂O diethyl ether
EtOH ethanol
EtOAc, EA, AcOEt ethyl acetate
h hour(s)
HPLC high performance liquid chromatography
IC₅₀ 50% inhibition concentration
iPrOH isopropyl alcohol or isopropanol
LCMS Liquid chromatography mass spectroscopy
LDA lithium di-isopropyl amide
Me methyl
MeOH methanol
NaBH(OAc)₃ sodium triacetoxyborohydride
NMR Nuclear Magnetic Resonance spectroscopy
Pd₂(dba)₃ Tris(dibenzylideneacetone)dipalladium(0)
PE petroleum ether or petrol
PPh₃ triphenylphosphine
Pr propyl
SFC supercritical fluid chromatography T3P 1-Propanephosphonic anhydride solution, 2,4,6-Tripropyl-1,3,5,2,4,6-trioxatriphosphorinane-2,4,6-trioxide
TFA trifluoroacetic acid
THE tetrahydrofuran
uv ultraviolet

1: LC-MS Method

Instrument: Agilent 1260 infinity HPLC with Agilent 6130 single quadrupole mass spec.

Column: Phenomenex Kinetex XB-C₁₈, 50×4.6 mm, 2.6 μm

Elution profile: See table below

| | % AQUEOUS (A) | |
|---|---|---|
| TIME | (0.1% FORMIC ACID | % ORGANIC (B) |
| (MINUTES) | IN WATER) | (100% ACETONITRILE) |
| 0 | 95 | 5 |
| 1.37 | 2 | 98 |
| 1.60 | 2 | 98 |
| 1.83 | 95 | 5 |
| 2.25 | 95 | 5 |

Flow rate: 2 ml/min; Detector wavelength: 225±50 nm bandwidth; Column temperature: 40° C.

Injection volume: 1 μl.

On occasion, a longer gradient using the same conditions but over 10 mins was also used.

Mass spec parameters: Scanning in ES+/−& APCI over 70-1000m/z; Needle wash: MeOH wash in vial 4, autosampler set up to do 5 needle washes (to wash the outside of the needle prior to injecting the sample; Sample preparation: 0.5-1.0 mg/ml in either acetonitrile or DMSO depending on the nature of the sample in terms of solubility.

2. Analytical NMR Method

BrukerAvance III 400 MHz with Ultrashield magnet & B-ACS-60 autosampler. ¹H and ¹³C NMR spectra were recorded in CDCl₃, DMSO-d₆ and D₂O. Chemical shifts were reported in ppm (δ) using Me₃Si as internal standard

3. Preparative HLPC Purification
Column Phenomenex Gemini C18, 5μ, 100×21.2 mm with Phenomenex Gemini C18, 15×21.2 mm Security Guard.
Flow Rate 20 ml/min (0-14.0 mins, then 1 ml/min)
Inj Vol 500 μl of DMSO solution at approximately 25 mg/ml concentration
Mobile Phase A: 0.1% TFA in water
   B: Acetontrile
Run Time 14 mins
Gradient

| Time (mins) | % A | % B |
|---|---|---|
| 0 | 90 | 10 |
| 0.6 | 90 | 10 |
| 9.0 | 60 | 40 |
| 10.5 | 0 | 100 |
| 12.0 | 0 | 100 |
| 12.2 | 90 | 10 |
| 14.0 | 90 | 10 |
| 14.4 | 90 | 10 |
| 14.5 | 90 | 10 |

4. Lyophilisation

Freeze drying was performed a Mechatech LyoDry Compact (−55C condenser) lyophiliser.

### Part 1: Synthesis of 1,6-Anhydro-N-acetylmuramic acid

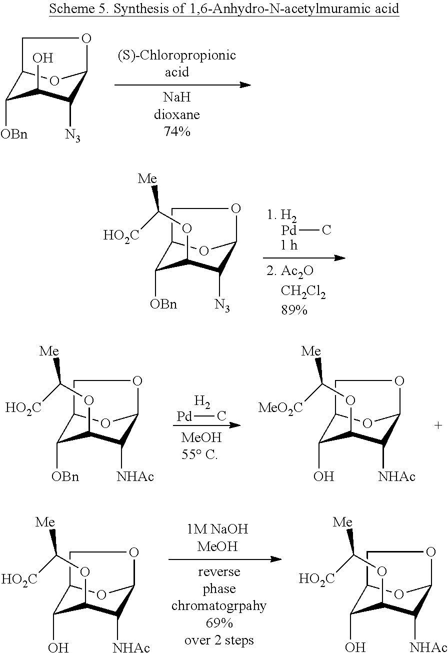

### Step 1. Preparation of (R)-2-(((1R,2S,3R,4R,5R)-4-Azido-2-(benzyloxy)-6,8-dioxabicyclo[3.2.1]octan-3-yl)oxy) propanoic acid

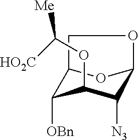 [SMILES: C[C@@H](O[C@H]1C(N=[N+]=[N-])[C@@H]2OCC(O2)[C@H]1OCc1ccccc1)C(=O)O]

To a solution of (1R,2S,3R,4R,5R)-4-azido-2-(benzyloxy)-6,8-dioxabicyclo[3.2.1]octan-3-ol (200 mg, 0.72 mmol) in anhydrous 1,4-dioxane (4 mL) was added sodium hydride (60% dispersion in oil; 191 mg, 4.76 mmol) and the mixture was heated at 45° C. for 10 min. The suspension was cooled to room temperature, (S)-2-chloropropioninc acid (188 mg, 148 μL, 1.73 mmol) was added and the mixture was heated at 90° C. for 2 h. The brown suspension was cooled to room temperature and concentrated to give a pale brown solid. This material was cautiously quenched with water (10 mL) and the solution was acidified to pH 3 with conc. hydrochloric acid. The aqueous suspension was extracted with dichloromethane (7×10 mL). The combined organic layers were washed with water (25 mL), dried (MgSO₄) and concentrated to give a green oil. This material was purified using a Biotage Isolera automated chromatography system under normal phase conditions (silica column, gradient of 0→100% methanol in dichloromethane) with detection at 254 nm to give titled acid (185 mg, 74%) as a green oil. R_{f}=0.40 (methanol-dichloromethane, 8:92 v/v)

¹H NMR (400 MHz, CDCl₃) δ 7.38 (m, 5H, 5×ArH), 5.57 (m, 1H, CH), 4.69 (m, 3H, CH and benzylic CH₂), 4.00 (m, 2H, 2×CH), 3.76 (dd, J=7.5, 5.6 Hz, 1H, CH), 3.63 (m, 1H, CH), 3.38 (m, 1H, CH), 3.31 (m, 1H, CH), 1.40 (d, J=6.9 Hz, 3H, CH₃).

### Step 2. Preparation of (R)-2-(((1R,2S,3R,4R,5R)-4-Acetamido-2-(benzyloxy)-6,8-dioxabicyclo[3.2.1]octan-3-yl)oxy) propanoic acid

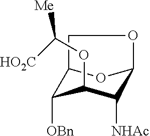 [SMILES: CC(=O)NC1[C@@H]2OCC(O2)[C@@H](OCc2ccccc2)[C@H]1O[C@H](C)C(=O)O]

To suspension of 10% Pd—C(19 mg, 10% w/w) in anhydrous methanol (1 mL) under nitrogen was added a solution of Step 1 acid (185 mg, 0.53 mmol) in anhydrous methanol (3 mL) and the reaction mixture was stirred under a hydrogen atmosphere for 1 h 15 min. The suspension was filtered through Celite and the filtrate was concentrated to give a white solid (172 mg). This material was dissolved in anhydrous dichloromethane (3.8 mL), acetic anhydride (1.56 mL) was added and the reaction mixture was stirred at room temperature overnight. The solution was concentrated and the residue was partitioned between dichloromethane (10 mL) and water. The layers were separated and the aqueous layer was extracted with dichloromethane (2×10 mL). The combined organic layers were washed with saturated brine (25 mL), dried (MgSO₄) and concentrated to give colourless oil. This material was purified using a Biotage Isolera automated chromatography system under normal phase conditions (silica column, gradient of 0→100% methanol in dichloromethane) with detection at 254 nm to give titled acid (173 mg, 89%) as a white foam.

LCMS r.t.=7.5 min, ESI-MS (m/z): 364 [M−H]⁻

¹H NMR (400 MHz, CDCl₃) δ 7.34 (m, 5H, 5×ArH), 6.17 (d, J=6.9 Hz, 1H, NH), 5.34 (m, 1H, CH), 4.61 (m, 3H, CH and benzylic CH₂), 4.23 (q, J=6.8 Hz, 1H, CH), 4.12 (m, 2H, 2×CH), 3.72 (dd, J=7.3, 5.9 Hz, 1H, CH), 3.47 (m, 1H, CH), 3.39 (m, 1H, CH), 1.94 (s, 3H, CH₃), 1.41 (d, J=6.9 Hz, 3H, CH₃).

### Step 3. Preparation of 1,6-Anhydro-N-acetylmuramic acid

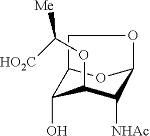 [SMILES: CC(=O)NC1[C@@H]2OCC(O2)[C@@H](O)[C@H]1O[C@H](C)C(=O)O]

To suspension of 10% Pd—C (26 mg, 15% w/w) in methanol (1 mL) under nitrogen was added a solution of Step 2 acid (171 mg, 0.47 mmol) in methanol (3 mL) and the reaction mixture was heated under hydrogen atmosphere at 55° C. for 4 h. The suspension was cooled to room temperature, filtered through Celite and the filtrate was concentrated to give a white solid (136 mg, mixture of 1,6-anhydro-N-acetylmuramic acid and 1,6-anhydro-N-acetylmuramic acid methyl ester). This material was dissolved in methanol (4 mL), 1 M aqueous solution of NaOH (4 mL) was added and the mixture was stirred for 1 h at room temperature. The methanol was removed and the aqueous solution was acidified to pH 3 with conc. hydrochloric acid and concentrated. The material was purified using a Biotage Isolera automated chromatography system under reversed-phase conditions (C18 column, gradient of 10→100% acetonitrile in water) with detection at 210 nm to give 1,6-anhydro-N-acetylmuramic acid (87 mg, 67%) as a white solid.

LCMS r.t.=1.3 min ESI-MS (m/z): 276.00 [M+H]⁺

¹H NMR (300 MHz, CD₃CN) δ 6.76 (d, J=8.7 Hz, 1H, NH), 5.51 (m, 1H, CH), 4.68 (m, 1H, CH), 4.44 (q, J=6.9 Hz, 1H, CH), 4.30 (dd, J=7.4, 1.0 Hz, 1H, CH), 4.05 (m, 1H, CH), 3.85 (m, 2H, 2×CH), 3.56 (m, 1H, CH), 2.10 (s, 3H, CH₃), 1.56 (d, J=6.9 Hz, 3H, CH₃).

### Part 2: Synthesis of ((R)-2-(((1R,2S,3R,4R,5R)-4-acetamido-2-hydroxy-6,8-dioxabicyclo[3.2.1]octan-3-yl)oxy)propanoyl)-L-alanine, (1,6-Anhydro-N-acetyl muramic acid-L-alanine)

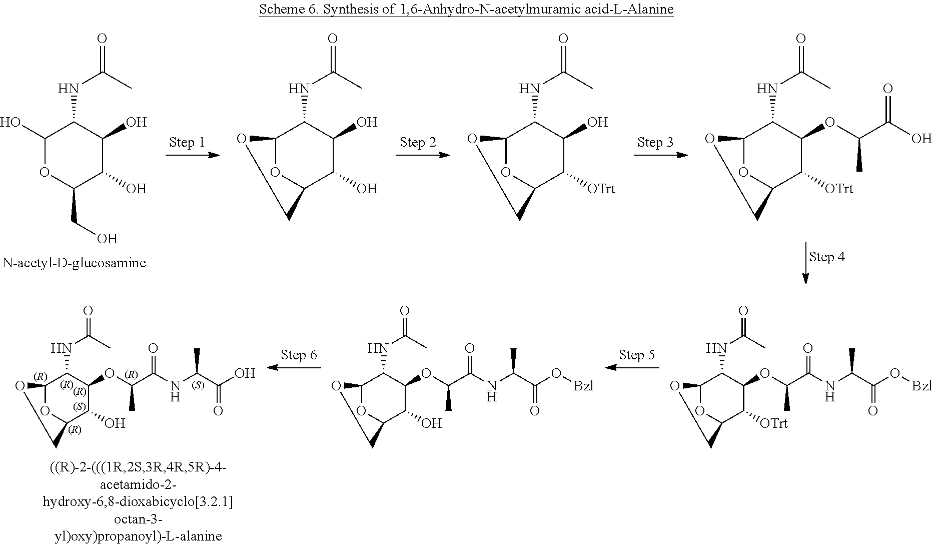

### Step 1. Preparation of N-((1R,2S,3R,4R,5R)-2,3-dihydroxy-6,8-dioxabicyclo[3.2.1]octan-4-yl)acetamide

To a stirred suspension of N-acetyl-D-glucosamine (30.0 g, 136 mmol) in pyridine (300 ml) at 0-5° C. was added dropwise a solution of 4-toluenesulphonyl chloride (31.0 g, 163 mmol) in pyridine (200 ml) over 1 hour. The reaction was stirred at 0-5° C. for 4 hours. MeOH (20 ml) was added in one portion and the solvent was removed in vacuo. The resulting brown viscous oil was taken up in ethanol (1 L) and DBU (52.5 mL, 407 mmol) was added in one portion. The mixture was stirred overnight at RT, and then concentrated in vacuo (water bath temperature <50° C.). The brown viscous oil residue was purified in two equal batches by flash chromatography on silica (eluent: 5% then 10% MeOH in EtOAc). Fractions containing mainly desired product (as judged by TLC) were combined and concentrated in vacuo to afford an off-white solid. NMR indicates that this material contains a minor aromatic impurity. The material was recrystallized from MeOH. After cooling in an ice bath, the title compound was isolated by vacuum filtration as a white solid, washed with a little cold MeOH and dried in a vacuum oven for 3 h at 40° C. (13.8 g, 50.6% yield).

¹H NMR (400 MHz, methanol-d4) δ5.25 (s, 1H), 4.48 (d, J=5.6 Hz, 1H), 4.18 (dd, J=7.2, 0.8 Hz, 1H), 3.83 (s, 1H), 3.68 (dd, J=7.2, 6.0 Hz, 1H), 3.56-3.54 (m, 2H), 1.98 (s, 3H)

### Step 2. Preparation of N-((1R,2S,3R,4R,5R)-3-hydroxy-2-(trityloxy)-6,8-dioxabicyclo[3.2.1]octan-4-yl)acetamide

To a suspension of Step 1 amide (8.5 g, 41.8 mmol) in DCM (300 ml) at RT was added 2,4,6-trimethylpyridine (10.15 g, 83.5 mmol) in one portion. To a solution of triphenylmethanol (16.3 g, 62.5 mmol) in DCM (200 ml) was added TMSOTf (12.1 ml, 62.5 mmol) in one portion under nitrogen. The resulting orange/brown solution and added dropwise over 15 mins to the amide solution with stirring under a nitrogen atmosphere. After 1 hour the reaction was quenched with pyridine (10 ml) followed by methanol (20 ml), and chloroform (800 ml) was added. The solution was extracted with water (300 ml), 1 M HCl (300 ml×2), saturated NaHCO₃ solution (300 ml), and water (300 ml). The organic phase was dried over sodium sulphate, filtered and concentrated in vacuo to afford an off white solid. This was purified by flash column chromatography on silica (eluent 1:1 EtOAc-hexane until elution of TrOH by product, then 100% EtOAc). Pure fractions (as judged by TLC) were combined and concentrated in vacuo to give a white foam.

This procedure was repeated in an identical fashion, and the products combined.

The combined product was triturated with 1:1 diethyl ether-hexane (100 ml) to afford the title compound as a white powder solid that was isolated by vacuum filtration and dried overnight in a 40° C. vacuum oven (16.8 g, 45% yield over two batches)

¹H NMR (400 MHz, acetone-d6) δ 7.57-7.54 (m, 6H), 7.38-7.33 (m, 6H), 7.31-7.27 (m, 3H), 6.66 (d, J=8.5 Hz, 1H), 5.18 (s, 1H), 4.08 (d, J=4.4 Hz, 1H), 3.93 (dd, J=7.2, 0.8 Hz, 1H), 3.75-3.71 (m, 2H), 3.66 (s, 1H), 3.35-3.34 (m, 1H), 3.29 (dd, J=6.8, 6.0 Hz, 1H), 1.95 (s, 3H)

### Step 3. Preparation of (R)-2-(((1R,2S,3R,4R,5R)-4-acetamido-2-(trityloxy)-6,8-dioxabicyclo[3.2.1]octan-3-yl)oxy)propanoic acid

To a stirred solution of Step 2 amide (4.00 g, 8.98 mmol) in anhydrous dioxane (60 ml) at RT was added NaH (60% in dispersion oil) (2.32 g, 60.6 mmol) in small portions over 15 minutes under an atmosphere of nitrogen. The resulting suspension was heated to 45° C. for 10 minutes and allowed to cool to RT. (2S)-2-chloropropanoic acid (2.06 mL, 22.4 mmol) was added dropwise via syringe over 10 minutes under nitrogen. The mixture was then heated to 90° C. for 1.5 hours and allowed to cool to RT. The solvent was removed in vacuo. To the residue was added ice cold water (100 ml) initially dropwise until effervescence stopped. The resulting solution was acidified to pH 3 with 2M HCl solution, precipitating a thick white solid. This was extracted into EtOAc (2×100 ml). The extracts were dried over sodium sulphate, filtered, and concentrated in vacuo to afford an off-white foamy solid. The crude product was purified by flash column chromatography on silica (eluent 5% MeOH in EtOAc+0.1% AcOH). Pure fractions (as judged by TLC analysis) were combined and concentrated in vacuo to afford a white foam that was triturated with 5% diethyl ether in hexane to afford the title compound as a white powder solid (3.98 g, 85% yield). [α]=−51.3° (c=0.0115 in chloroform, 20.8° C.).

LCMS: 96.7% purity; RT=1.720 min; m/z calcd for C₃₀H₃₁NO₇ [M−H]⁻ 516, found 516.

¹H NMR (400 MHz, DMSO-d6) δ 12.14 (s, 1H), 7.60 (br d, J=6.4 Hz, 1H), 7.48-7.46 (m, 6H), 7.37-7.27 (m, 9H), 5.22 (s, 1H), 3.74-3.68 (m, 3H), 3.59 (d, J=6.0 Hz), 3.49 (s, 1H), 3.32 (s, 1H), 3.25-3.33 (m, 1H, obscured by solvent water peak), 1.93 (s, 3H), 1.06 (d, J=6.8 Hz, 3H);

### Step 4. Preparation of benzyl ((R)-2-(((1R,2S,3R,4R,5R)-4-acetamido-2-(trityloxy)-6,8-dioxabicyclo[3.2.1]octan-3-yl)oxy)propanoyl)-L-alaninate

Step 3 acid (8.00 g, 15.5 mmol) and L-Alanine-OBzl (3.67 g, 17.0 mmol) were dissolved in DMF (150 ml, 0.1 M) at room temperature. DIEA (8.88 mL, 51.0 mmol) and HATU (6.47 g, 17.0 mmol) were then added and the mixture was stirred for 18 hours at RT. The reaction mixture was added to ice water (2 L) and the resulting white precipitate was extracted with 20% EtOAc in ether (3×300 ml). The combined extracts were washed with brine (2×300 ml) and dried over sodium sulphate, filtered, and concentrated in vacuo to afford a white foam. This was purified by flash column chromatography on silica (eluent: 50% to 75% EtOAc in hexane gradient). Pure fractions by TLC were combined and concentrated in vacuo. The white foam product was triturated with 10% EtOAc in diethyl ether and isolated by vacuum filtration to afford the title compound as a white solid (9.13 g, 87%).

LCMS: 100% purity; RT=6.55 min; m/z calcd for C₄₀H₄₂N₂O₈ [M−H]⁻ 677, found 677.

¹H NMR (400 MHz, CDCl3) δ 7.85 (d, J=8.0 Hz), 7.50-7.47 (m, 6H), 7.36-7.29 (m, 14H), 5.37 (s, 1H), 5.15-5.07 (m, 2H), 4.64-4.56 (1H, m), 4.04 (d, J=7.6 Hz, 1H), 3.98 (d, J=9.2 Hz, 1H), 3.78-3.73 (m, 2H), 3.68 (s, 1H), 3.45 (dd, J=7.6 Hz and 6.0 Hz, 1H), 3.12 (s, 1H), 2.01 (s, 3H), 1.39 (d, J=7.2 Hz, 3H), 1.15 (d, J=6.8 Hz, 3H)

### Step 5. Preparation of benzyl ((R)-2-(((1R,2S,3R,4R,5R)-4-acetamido-2-hydroxy-6,8-dioxabicyclo[3.2.1]octan-3-yl)oxy)propanoyl)-L-alaninate

To a solution of Step 4 ester (8.50 g, 12.5 mmol) in DCM (165 ml) was added TFA (9.30 mL, 125 mmol) in one portion at RT. A yellow colour developed immediately. The solution was stirred for 3 hours at RT. The solution was added very slowly into stirred conc. NaHCO₃ solution (500 ml) and extracted with DCM (2×250 ml). The crude product was purified by flash column chromatography on silica (eluent: 5-10% MeOH in EtOAc). Pure fractions (as judged by TLC) combined to afford the title compound as a white foam (5.05 g, 92% yield).

LCMS: 98.1% purity; RT=1.402 min; m/z calcd for C₂₁H₂₈N₂O₈ [M+H]⁺ 437, found 437.

¹H NMR (400 MHz, CDCl3) δ 7.84 (d, J=8.0 Hz, 1H), 7.37-7.31 (m, 5H), 6.22 (d, J=9.2 Hz, 1H), 5.41 (s, 1H), 5.20-5.12 (m, 2H), 4.67-4.59 (1H, m), 4.52 (d, J=5.2 Hz, 1H), 4.26 (d, J=7.2 Hz, 1H), 4.17-4.05 (m, 2H), 3.99 (d, J=9.2 Hz, 1H), 3.76-3.73 (m, 2H), 3.44 (s, 1H), 2.86 (br s, 1H), 1.98 (s, 3H), 1.44 (d, J=7.2 Hz, 3H), 1.38 (d, J=6.8 Hz, 3H).

### Step 6. Preparation of ((R)-2-(((1R,2S,3R,4R,5R)-4-acetamido-2-hydroxy-6,8-dioxabicyclo[3.2.1]octan-3-yl)oxy)Propanoyl)-L-alanine

To a solution of Step 5 ester (3.58 g, 8.20 mmol) in EtOH (100 ml) was added Pd/C (10.0%, 0.437 g, 0.410 mmol) moistened with 4 drops of water. The mixture was stirred under hydrogen (50 PSI) overnight at RT in a steel autoclave. The reaction mixture was filtered through a short pad of celite and the filter cake washed with ethanol. The filtrate was concentrated in vacuo to afford a colourless viscous glass solid. This material was triturated with diethyl ether (with sonication). The precipitate was isolated by vacuum filtration and dried to constant mass in a 40° C. vacuum oven to afford the title compound as a white powder solid (2.62 g, 92%). [α]=−49.4° (c=0.0115 in MeOH, 20.8° C.).

LCMS: 100% purity; RT=0.963 min; m/z calcd for C₁₄H₂₂N₂O₈ [M+H]⁺ 347; ¹H NMR (400 MHz, DMSO-d6) δ 12.54 (br s, 1H), 7.70 (d, J=7.6 Hz, 1H), 7.63 (d, J=8.8 Hz, 1H), 5.29 (s, 1H), 5.24 (br s, 1H), 4.48 (d, J=5.2 Hz, 1H), 4.29-4.23 (m, 1H), 4.12-4.02 (m, 2H), 3.69 (d, J=8.8 Hz, 1H), 3.61 (dd, J=7.2 Hz and 6.0 Hz, 1H), 3.53 (s, 1H), 3.26 (signal obscured by solvent water peak), 1.98 (s, 3H), 1.29 (d, J=7.2 Hz, 3H), 1.24 (d, J=6.8 Hz, 3H). found 347.

### Part 3; Preparation of Linkers

### (a) Synthesis of Linker-1

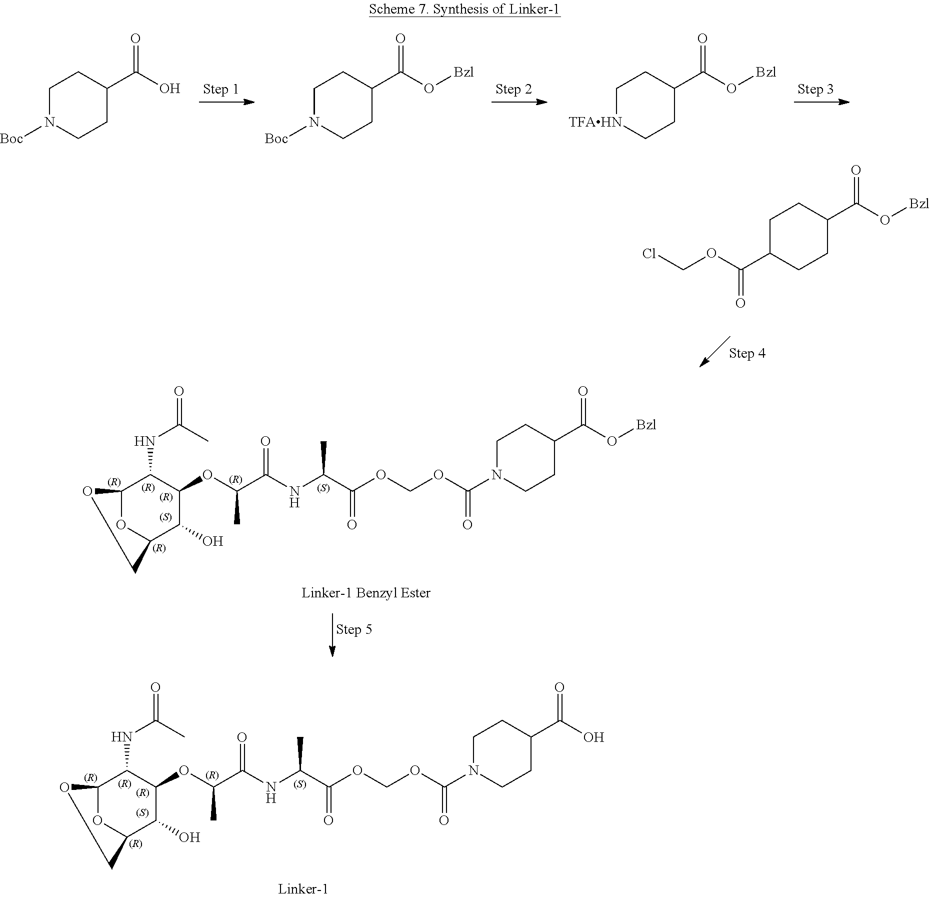

### Step 1. Preparation of 4-benzyl 1-(tert-butyl) piperidine-1,4-dicarboxylate

To a suspension of 1-tert-butoxycarbonylpiperidine-4-carboxylic acid (7.00 g, 30.5 mmol) in DMF (70 ml) was added K₂CO₃ (11.0 g, 79.4 mmol) followed by benzylchloride (5.02 g, 39.7 mmol). The reaction was stirred at RT for 4 days. The mixture was poured into cold water (1.5 L) and extracted with diethyl ether (2×300 ml). The extracts were washed with brine and concentrated in vacuo to afford a colourless oil. The crude product was purified by flash column chromatography on silica (eluent: 10-50% EtOAc in hexane gradient) to afford the title compound as colourless oil (9.50 g, 97% yield).

LCMS: 95% purity; RT=6.20 min; m/z calcd for C₁₈H₂₅NO₄ [M+H-Boc]⁺ 220; ¹H NMR (400 MHz, CDCl3) δ 7.38-7.32 (m, 5H), 5.12 (s, 2H), 4.01 (br d, J=10.4 Hz, 2H), 2.82 (t, J=12 Hz, 2H), 2.53-2.46 (m, 1H), 1.88 (m, 2H), 1.69-1.59 (m, 2H), 1.45 (s, 9H)

### Step 2. Preparation of benzyl piperidine-4-carboxylate.trifluoroacetate

To a stirred solution of Step 1 ester (9.40 g, 29.4 mmol) in DCM (120 ml) was added TEA (13.1 mL, 177 mmol) in one portion. The resulting yellow solution was stirred for 18 h. The solvent was removed in vacuo and the residue co-evaporated with toluene (2×50 ml) to remove TEA traces. The title compound (pale yellow viscous oil, 11.8 g, 120%) was used in next step without purification.

LCMS: 83% purity; RT=2.81 min; m/z calcd for free base C₁₃H₁₇NO₂ [M+H]⁺ 220, found 220.

### Step 3. Preparation of 4-benzyl 1-(chloromethyl) piperidine-1,4-dicarboxylate

To a stirred solution of Step 2 salt (3.27 g, 9.81 mmol) in DCM (60 ml) was added TEA (2.73 mL, 19.6 mmol) in one portion. The resulting yellow solution was cooled in an ice/water bath and chloromethyl carbonochloridate (0.960 mL, 10.8 mmol) was added dropwise over 15 mins with stirring under an atmosphere of nitrogen. The mixture was allowed to warm to RT and stirred for 18 hours. The solvent was removed in vacuo and the residue taken up in DCM (150 ml). This solution was washed with 1M HCl (50 ml) and concentrated sodium bicarbonate solution (50 ml) and dried over sodium sulphate. The solution was concentrated in vacuo to afford the title compound as a colourless viscous oil (2.35 g, 76%).

LCMS: 97% purity; RT=5.71 min; m/z calcd C₁₅H₁₈ClNO₄ [M+H]⁺312, found 312; ¹H NMR (400 MHz, CDCl3) δ 7.38-7.30 (m, 5H), 5.16 (d, J=7.6 Hz, 2H), 5.13 (s, 2H), 4.13-3.99 (m, 2H), 3.10-2.91 (m, 2H), 2.60-2.50 (m, 1H), 2.03-1.85 (m, 2H), 1.85-1.61 (m, 2H)

### Step 4. Preparation of Linker-1 Benzyl Ester

To a suspension of Step 3 ester (0.250 g, 0.722 mmol) and Cs₂CO₃ (0.259 g, 0.794 mmol) in anhydrous DMF (4 ml) was added Part 2 Step 6 acid ((R)-2-(((1R,2S,3R,4R,5R)-4-acetamido-2-hydroxy-6,8-dioxabicyclo[3.2.1]octan-3-yl)oxy)propanoyl)-L-alanine) (0.225 g, 0.722 mmol) in one portion.. The reaction mixture was poured into cold water (100 ml) and extracted with EtOAc (3×30 ml). The extracts were washed with brine (2×50 ml) and dried over sodium sulphate. The crude product was purified by flash column chromatography on silica (eluent: 5-10% MeOH in EtOAc) to afford the title compound as a colourless viscous oil (259 mg, 57%).

LCMS: 100% purity; RT=4.85 min; m/z calcd C₂₉H₃₉N₃O₁₂ [M+H]⁺ 622, found 622; ¹H NMR (400 MHz, CDCl3) δ 7.73 (d, J=7.6 Hz, 1H), 7.38-7.30 (m, 5H), 6.19 (d, J=9.2 Hz, 1H), 5.79 (br s, 1H), 5.72 (d, J=5.6 Hz, 1H), 5.41 (s, 1H), 5.12 (s, 2H), 4.57 (d, J=5.6 Hz, 1H), 4.50-4.42 (m, 1H), 4.28 (d, J=7.6 Hz, 1H), 4.09-4.02 (m, 4H), 3.81 (dd, J=7.2 Hz and 5.6 Hz, 1H), 3.74 (s, 1H), 3.46 (s, 1H), 3.15 (br d, J=10.4 Hz, 1H), 3.06-2.90 (m, 3H), 2.58-2.50 (m, 1H), 2.02-1.83 (m, 5H), 1.85-1.58 (m, 2H), 1.45 (d, J=7.2 Hz, 3H), 1.36 (d, J=6.8 Hz, 3H)

### Step 5. Preparation of Linker-1

To a solution of Step 4 ester (0.220 g, 0.354 mmol) in EtOH (10 ml) was added Pd/C (10.0%, 0.0377 g, 0.0354 mmol) moistened with a drop of water. The mixture was stirred under hydrogen (50 PSI) for 18 h at RT. The mixture was filtered through a pad of celite and the filter cake washed with EtOH (2×10 ml). The filtrate was concentrated in vacuo and the residue triturated with EtOAc (5 ml) to afford the title compound as a white solid (128 mg, 68%) LCMS: 100% purity; RT=1.19 min; m/z calcd C₂₂H₃₃N₃O₁₂ [M+H]⁺ 532, found 532.

¹H NMR (400 MHz, MeOD-d4) δ 8.19 (d, J=7.6 Hz, 1H), 5.88-5.73 (m, 2H), 5.39 (s, 1H), 4.56 (d, J=5.2 Hz, 1H), 4.51-4.42 (m, 2H), 4.25 (d, J=7.2 Hz, 1H), 4.20-4.15 (m, 1H), 4.00 (br d, J=13.6 Hz, 2H), 3.09 (s, 1H), 3.72 (dd, J=7.6 and 6.0 Hz, 1H), 3.69 (d, J=1.2 Hz, 1H), 3.41-3.40 (m, 1H), 2.57-2.49 (m, 1H), 1.90 (s, 3H), 2.00-1.82 (br s, 1H), 1.68-1.50 (m, 2H), 1.43 (d, J=7.2 Hz, 3H), 1.35 (d, J=6.8 Hz, 3H)

### (b) Synthesis of Linker-2 and Linker 3

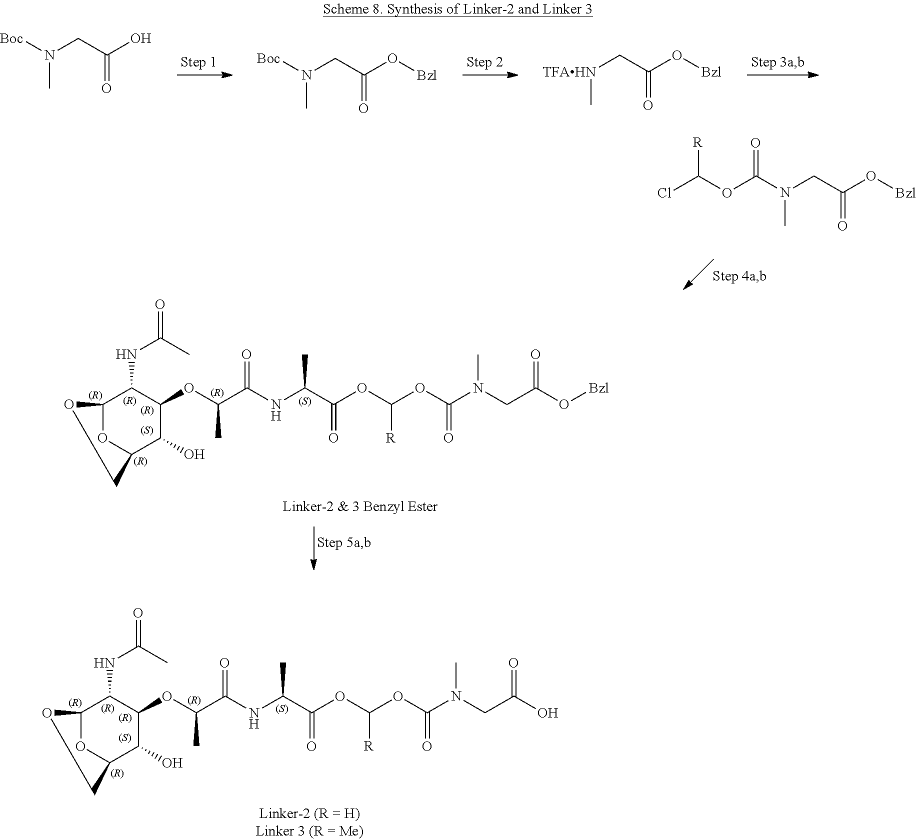

### Step 1. Preparation of Benzyl N-(tert-butoxycarbonyl)-N-methylglycinate

To a suspension of 2-[tert-butoxycarbonyl(methyl)amino]acetic acid (7.00 g, 37.0 mmol) in DMF (70 ml) was added K₂CO₃ (13.3 g, 96.2 mmol) followed by benzylbromide (8.23 g, 48.1 mmol). The reaction was stirred at RT for 3 days. The mixture was poured into cold water (1.5 L) and extracted with diethyl ether (2×200 ml). The extracts were washed with brine and concentrated in vacuo to afford a colourless oil. The crude product was purified by flash column chromatography on silica (eluent: 10-50% EtOAc in hexane gradient) to afford the title compound as colourless oil (10.0 g, 96.8% yield).

LCMS: 100% purity; RT=1.77 min; m/z calcd for C₁₅H₂₁NO₄ [M+H-Boc]⁺ 180; ¹H NMR (400 MHz, CDCl3) δ 7.42-7.29 (m, 5H), 5.17 (d, J=3.6 Hz, 2H), 4.02 (s, 1H), 3.93 (s, 1H), 2.92 (d, J=9.6 Hz, 3H), 1.46-1.37 (app d, 9H)

### Step 2. Preparation of Benzyl methylglycinate. Trifluoroacetate

To a stirred solution of Step 1 ester (2.00 g, 7.16 mmol) in DCM (30 ml) was added TFA (3.19 mL, 43.0 mmol) in one portion. The resulting yellow solution was stirred for 18 h. The solvent was removed in vacuo and the residue co-evaporated with toluene (2×20 ml) to remove TFA traces. The title compound, as a colourless viscous oil, (2.69 g, 128%) was used in next step without purification.

LCMS: 92.8% purity; RT=0.87 min; m/z calcd for free base C₁₀H₁₃NO₂ [M+H]⁺ 180, found 180; ¹H NMR (400 MHz, CDCl3) δ 7.40-7.25 (m, 5H), 5.20 (s, 2H), 5.10-4.00 (br s, 2H), 3.87 (s, 2H), 2.80 (s, 3H)

### Step 3a. Preparation of Benzyl N-((chloromethoxy)carbonyl)-N-methylglycinate

To a stirred solution of Step 2 salt (2.10 g, 7.16 mmol) in DCM (30 ml) was added TEA (2.00 mL, 14.3 mmol) in one portion. The resulting yellow solution was cooled in an ice/water bath and chloromethyl carbonochloridate (0.700 mL, 7.88 mmol) was added dropwise over 15 mins with stirring under an atmosphere of nitrogen. The mixture was allowed to warm to RT and stirred for 18 hours. The solvent was removed in vacuo and the residue taken up in DCM (150 ml). This solution was washed with 1M HCl (50 ml) and concentrated sodium bicarbonate solution (50 ml) and dried over sodium sulphate. The solution was concentrated in vacuo to afford the title compound as a colourless viscous oil (1.49 g, 76%).

LCMS: 98.7% purity; RT=5.32 min; m/z calcd for C₁₂H₁₄ClNO₄ [M+H]⁺ 272, found 272; ¹H NMR (400 MHz, CDCl3) δ 7.42-7.28 (m, 5H), 5.79 (s, 1H), 5.71 (s, 1H), 5.18 (d, J=2.4 Hz, 2H), 4.10 (s, 1H), 4.04 (s, 1H), 3.02 (d, J=8.8 Hz, 3H).

### Step 3b. Preparation of Benzyl N-((1-chloroethoxy)carbonyl)-N-methylglycinate

To a stirred solution of Step 2 ester (2.50 g, 8.95 mmol) in DCM (30 ml) was added TFA (3.99 mL, 53.7 mmol) in one portion. The resulting yellow solution was stirred for 18 h. The solvent was removed in vacuo and the residue co-evaporated with toluene (2×20 ml) to remove TFA traces. The crude TFA salt product was taken up in DCM (50 ml) and TEA (2.49 mL, 17.9 mmol) was added in one portion. The solution was cooled in an ice/water bath and 1-chloroethyl carbonochloridate (1.06 mL, 9.84 mmol) was added dropwise over 15 mins with stirring under an atmosphere of nitrogen. The mixture was allowed to warm to RT and stirred for 18 hours. The solvent was removed in vacuo and the residue taken up in DCM (150 ml). This solution was washed with 1M HCl (50 ml) and concentrated sodium bicarbonate solution (50 ml) and dried over sodium sulphate. The solution was concentrated in vacuo to afford the title compound as a pale brown viscous oil (2.43 g, 95%).

¹H NMR (400 MHz, CDCl3) δ 7.42-7.28 (m, 5H), 6.58-6.50 (m, 1H), 5.23-5.15 (m, 2H), 4.27-4.12 (m, 1H), 3.97-3.89 (m, 1H), 3.01 (d, J=7.2 Hz, 3H), 1.82 (d, J=6.0 Hz, 1.5H), 1.68 (d, J=5.6 Hz, 1.5H)

### Step 4a. Preparation of (((2-(benzyloxy)-2-oxoethyl)(methyl)carbamoyl)oxy)methyl ((R)-2-(((1R,2S,3R,4R,5R)-4-acetamido-2-hydroxy-6,8-dioxabicyclo[3.2.1]octan-3-yl)oxy)propanoyl)-L-alaninate

To a suspension of Part 2 Step 6 acid (0.150 g, 0.43 mmol) and Cs₂CO₃ (0.155 g, 0.47 mmol) in anhydrous DMF (2.5 ml) was added Step 3a chloride (0.129 g, 0.47 mmol) in one portion. The suspension was stirred for 20 hours at RT. The reaction mixture was poured into cold water (60 ml) and extracted with EtOAc (3×30 ml). The extracts were washed with brine (2×50 ml) and dried over sodium sulphate. The crude product was purified by flash column chromatography on silica (eluent: 5-10% MeOH in EtOAc) to afford the title compound as a colourless viscous oil (168 mg, 66%).

LCMS 97.0% purity; RT=1.45 min; m/z calcd for C₂₆H₃₅N₃O₁₂ [M+H]⁺ 582, found 582; ¹H NMR (400 MHz, CDCl3) δ 8.01 (br s, 1H), 7.75-7.33 (m, 5H), 6.19 (d, J=9.2 Hz, 0.5H), 6.12 (d, J=9.2 Hz, 0.5H), 5.79 (s, 1H), 5.72 (q, J=5.6 Hz, 1H), 5.41 (s, 1H), 5.18 (app d, J=8.4 Hz, 2H), 4.60-4.47 (m, 2H), 4.32-4.27 (m, 1H), 4.20-3.95 (m, 5H), 3.80 (dd, J=7.6 Hz and 6.0 Hz, 1H), 3.74 (d, J=7.6 Hz, 1H), 3.44 (d, J=8.4 Hz, 1H), 2.99 (s, 3H), 2.78 (br s, 1H), 1.98 (s, 3H), 1.47-1.43 (m, 3H), 1.43-1.38 (m, 3H)

### Step 4b. Preparation of 1-(((2-(benzyloxy)-2-oxoethyl)(methyl)carbamoyl)oxy)ethyl ((R)-2-(((1R,2S,3R,4R,5R)-4-acetamido-2-hydroxy-6,8-dioxabicyclo[3.2.1]octan-3-yl)oxy)propanoyl)-L-alaninate

To a suspension of Part 2 Step 6 acid (0.150 g, 0.433 mmol) and Cs₂CO₃ (0.169 g, 0.520 mmol) in anhydrous DMSO (0.5 ml) was added Step 3b chloride (0.148 g, 0.520 mmol) in one portion. The suspension was stirred for 20 hours at RT. The reaction mixture was poured into cold water (50 ml) and extracted with EtOAc (4×30 ml). The extracts were washed with brine (2×50 ml) and dried over sodium sulphate. The crude product, a brown viscous oil, was purified by flash column chromatography on silica (eluent 5-10% MeOH in EtOAc) to afford a yellow brown viscous oil (81 mg, 31%).

LCMS 97.1% purity; RT=1.48 min; m/z calcd for C₂₇H₃₇N₃O₁₂ [M+H]⁺ 596, found 596; ¹H NMR (400 MHz, CDCl3) δ 7.88-7.76 (m, 1H), 7.42-7.30 (m, 5H), 6.90-6.73 (m, 1H), 6.17 (d, J=9.2 Hz, 1H), 5.41 (s, 1H), 5.17 (dd, J=8.8 Hz and 2.4 Hz, 2H), 4.62-4.45 (m, 2H), 4.38-4.30 (m, 2H), 4.22-4.08 (m, 2H), 4.08-3.90 (m, 2H), 3.85-3.73 (m, 2H), 3.44 (s, 1H), 2.70 (br s, 1H), 1.98 (s, 3H), 1.52 (app t, J=5.6 Hz, 2H), 1.44-1.37 (m, 8H)

### Step 5a. Preparation of Linker-2

To a solution of Step 4a ester (220 mg, 0.378 mmol) in EtOH (10 ml) was added Pd/C (10.0%, 40 mg, 0.0378 mmol) moistened with a drop of water. The mixture was stirred under hydrogen (50 PSI) overnight. The mixture was then filtered through a short pad of celite and the filter cake washed with EtOH. Filtrate concentrated in vacuo to afford the title compound as a colourless glassy solid (149 mg, 80%).

LCMS 100% purity by ELSD; RT=1.13 min; m/z calcd for C₁₉H₂₉N₃O₁₂ [M+H]⁺ 492, found 492; ¹H NMR (400 MHz, MeOD-d4) δ 5.88-5.72 (m, 2H), 5.39 (s, 1H), 4.56 (d, J=5.2 Hz, 1H), 4.51-4.43 (m, 1H), 4.25 (d, J=7.6 Hz, 1H), 4.21-4.15 (m, 1H), 4.01-4.00 (m, 2H), 3.90 (s, 1H), 3.75 (dd, J=7.2 Hz and 5.6 Hz, 1H), 3.69 (d, J=1.2 Hz, 1H), 3.40 (d, J=1.6 Hz, 1H), 2.97 (d, J=5.6 Hz, 3H), 1.97 (s, 3H), 1.44 (dd, J=7.6 and 3.6 Hz, 3H), 1.36 (d, J=6.8 Hz, 3H)

### Step 5b. Preparation of Linker-3

To a solution of Step 4b ester (0.190 g, 0.319 mmol) in EtOH (10 ml) was added Pd/C (10.0%, 0.0339 g, 0.0319 mmol) moistened with a drop of water. The mixture was stirred under hydrogen (50 PSI) overnight, and then filtered through a short pad of celite. Filter cake washed with EtOH (2×5 ml) and filtrate concentrated on vacuo. Residue triturated with ether (5 ml) to afford the title compound as off white solid (131 mg, 81%).

LCMS 100% purity by ELSD; RT=1.17 min; m/z calcd for C₂₀H₃₁N₃O₁₂ [M+H]⁺ 506, found 506; ¹H NMR (400 MHz, MeOD-d4) δ 6.79-6.75 (m, 1H), 5.38 (d, J=1.6 Hz, 1H), 4.56 (d, J=2.8 Hz, 1H), 4.52-4.35 (m, 1H), 4.25 (d, J=7.2 Hz, 1H), 4.23-4.13 (m, 1H), 4.12-3.85 (m, 3H), 3.80-3.72 (m, 1H), 3.69 (s, 1H), 3.40 (s, 1H), 2.98-2.95 (m, 3H), 1.57-1.28 (m, 9H).

### (c) Synthesis of Linker-4 and Linker 5

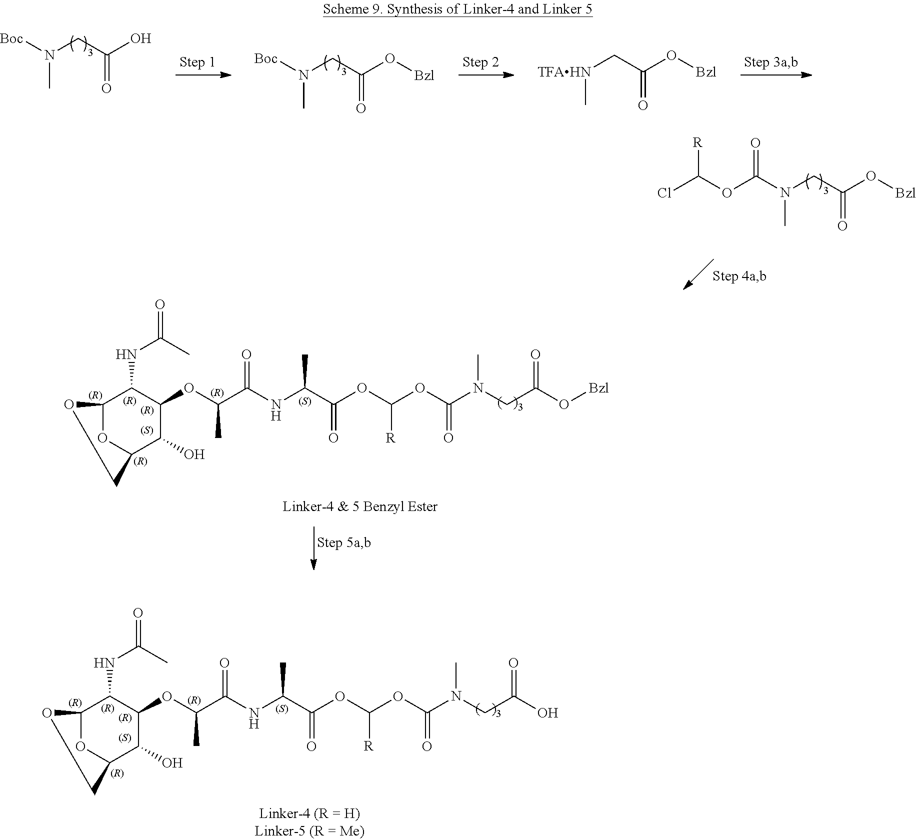

### Step 1. Preparation of Benzyl 4-((tert-butoxycarbonyl)(methyl)amino)butanoate

To a suspension of 4-[tert-butoxycarbonyl(methyl)amino]butanoic acid (2.50 g, 11.5 mmol) in DMF (25 ml) was added K₂CO₃ (4.13 g, 29.9 mmol) followed by benzylbromide (2.56 g, 15.0 mmol). The reaction was stirred at RT for 4 days. The mixture was poured into cold water (0.5 L) and extracted with diethyl ether (2×100 ml). The extracts were washed with brine and concentrated in vacuo to afford a colourless oil. The crude product was purified by flash column chromatography on silica (eluent: 10-50% EtOAc in hexane gradient) to afford the title compound as colourless viscous oil (3.29 g, 10.7 mmol, yield: 93.0

LCMS: 95.5% purity; RT=1.87 min; m/z calcd for C₁₇H₂₅NO₄ [M+H-Boc]⁺ 208; ¹H NMR (400 MHz, CDCl3) δ 7.40-7.28 (m, 5H), 5.12 (s, 2H), 3.24 (app t, J=6.8 Hz, 2H), 2.82 (s, 3H), 2.36 (app t, J=7.2 Hz, 2H), 1.85 (dq, J=7.2 Hz, 2H), 1.44 (s, 9H)

### Step 2. Preparation of Benzyl 4-(methylamino)butanoate. trifluoroacetate

To a stirred solution of Step 1 ester (3.29 g, 10.7 mmol) in DCM (50 ml) was added TFA (4.77 mL, 64.2 mmol) in one portion. The resulting yellow solution was stirred for 18 h. The solvent was removed in vacuo and the residue co-evaporated with toluene (2×20 ml) to remove TFA traces. The title compound (4.10 g, 119%) was obtained as a yellow oil. The material was used directly in Steps 3a and 3b without purification or characterisation.

### Step 3a. Preparation of Benzyl 4-(((chloromethoxy)carbonyl)(methyl)amino)butanoate

To a stirred solution of Step 2 salt (1.72 g, 5.35 mmol) in DCM (30 ml) was added TEA (2.24 mL, 16.1 mmol) in one portion. The resulting yellow solution was cooled in an ice/water bath and chloromethyl carbonochloridate (0.524 mL, 5.89 mmol) was added dropwise over 15 mins with stirring under an atmosphere of nitrogen. The mixture was allowed to warm to RT and stirred for 18 hours. The solvent was removed in vacuo and the residue taken up in EtOAc (150 ml). This solution was washed with 0.5 M HCl (50 ml) and concentrated sodium bicarbonate solution (50 ml) and dried over sodium sulphate. The solution was concentrated in vacuo. Purified by flash column chromatography on silica (eluent 20-50% EtOAc in hexane gradient) to afford the title compound as a colourless oil (0.92 g, 57% over two steps from CR-0056).

LCMS: 95.2% purity; RT=1.74 min; m/z calcd for C₁₄H₁₈ClNO₄ [M+H]⁺300, found 300; ¹H NMR (400 MHz, CDCl3) δ 7.42-7.26 (m, 5H), 5.71 (d, J=16.0 Hz, 2H), 5.12 (s, 2H), 3.50-3.20 (m, 2H), 3.01-2.80 (m, 3H), 2.42-2.28 (m, 2H), 2.00-1.80 (m, 2H)

### Step 3b. Preparation of Benzyl 4-(((1-chloroethoxy)carbonyl)(methyl)amino)butanoate

To a stirred solution of Step 2 salt (1.33 g, 4.13 mmol) in DCM (20 ml) was added DIEA (2.12 mL, 12.4 mmol) in one portion. The resulting yellow solution was cooled in an ice/water bath and 1-chloroethyl carbonochloridate (0.407 mL, 4.13 mmol) in DCM (5 ml) was added dropwise over 15 mins with stirring under an atmosphere of nitrogen. The mixture was allowed to warm to RT and stirred for 18 hours. The solvent was removed in vacuo and the residue taken up in DCM (150 ml). This solution was washed with water (100 ml) and dried over sodium sulphate. The crude product was purified by flash column chromatography on silica (eluent: 10-50% EtOAc in hexane) to afford the title compound (0.91 g, 70%) as colourless oil.

¹H NMR (400 MHz, CDCl3) δ 7.42-7.28 (m, 5H), 6.56 (app dq, J=5.6 Hz, 1H), 5.12 (s, 2H), 3.48-3.24 (m, 2H), 2.91 (app d, J=8.8 Hz, 3H), 2.44-2.30 (m, 2H), 1.98-1.82 (m, 2H), 1.78 (app dd, J=13.2 Hz and 6.0 Hz, 3H)

### Step 4a. Preparation of Benzyl 4-(((((((R)-2-(((1R,2S,3R,4R,5R)-4-acetamido-2-hydroxy-6,8-dioxabicyclo[3.2.1]octan-3-yl)oxy)propanoyl)-L-alanvl)oxy)methoxv)carbonvl)(methvl)amino)butanoate

To a suspension of Part 2 Step 6 acid (0.150 g, 0.433 mmol) and Step 3a chloride (0.260 g, 0.866 mmol) in anhydrous DMF (2 ml) was added DIEA (0.156 mL, 0.910 mmol) in one portion, followed by NaI (0.130 g, 0.866 mmol). The suspension was stirred for 48 hours at RT. The reaction mixture was poured into cold water (100 ml) and extracted with EtOAc (4×30 ml). The extracts were washed with brine (50 ml) and 5% LiCl solution (50 ml) and dried over sodium sulphate. The crude product, a brown viscous oil, was purified by flash column chromatography on silica (eluent 5-10% MeOH in EtOAc) to afford the title compound as colourless viscous oil (181 mg, 68%).

LCMS: 99.3% purity; RT=1.49 min; m/z calcd for C₂₈H₃₉N₃O₁₂ [M+H]⁺610, found 610; ¹H NMR (400 MHz, CDCl3) δ 7.71 (d, J=7.6 Hz, 1H), 7.42-7.28 (m, 5H), 6.20-6.10 (m, 1H), 5.82-5.66 (m, 2H), 5.41 (s, 1H), 5.12 (s, 2H), 4.55 (d, J=5.2 Hz, 1H), 4.52-4.42 (m, 1H), 4.28 (d, J=7.6 Hz, 1H), 4.10-3.98 (m, 2H), 3.34-3.22 (m, 2H), 3.46 (s, 1H), 3.40-3.15 (m, 2H), 2.97 (dd, J=16.5 Hz and 3.6 Hz, 1H), 2.89 (d, J=5.2 Hz, 3H), 2.44-2.30 (m, 2H), 1.97 (s, 3H), 1.88 (dd, J=7.2 Hz and 5.2 Hz, 3H), 1.40-1.30 (m, 3H).

### Step 4b. Preparation of Benzyl 4-(((1-((((R)-2-(((1R,2S,3R,4R,5R)-4-acetamido-2-hydroxy-6,8-dioxabicyclo[3.2.1]octan-3-yl)oxy)propanoyl)-L-alanvl)oxy)ethoxv)carbonvl)(methvl)amino)butanoate

To a suspension of Part 2 Step 6 acid (0.200 g, 0.577 mmol) and Step 3b chloride (0.362 g, 1.15 mmol) in anhydrous DMF (3 ml) was added DIEA (0.208 mL, 1.21 mmol) in one portion, followed by NaI (0.173 g, 1.15 mmol). The suspension was stirred for 24 hours at RT. The reaction mixture was poured into cold water (100 ml) and extracted with EtOAc (4×30 ml). The extracts were washed with brine (50 ml) and 5% LiCl solution (50 ml) and dried over sodium sulphate. The crude product, a brown viscous oil, was purified by flash column chromatography on silica (eluent 5-10% MeOH in EtOAc) to afford the title compound (0.11 g, 30%) as pale yellow viscous oil.

LCMS: 98.6% purity; RT=1.53 min; m/z calcd for C₂₉H₄₁N₃O₁₂ [M+H]⁺ 624, found 624; ¹H NMR (400 MHz, CDCl3) δ 7.86-7.78 (m, 1H), 7.42-7.28 (m, 5H), 6.90-6.74 (m, 1H), 6.10 (d, J=9.2 Hz, 1H), 5.41 (d, J=2.0 Hz, 1H), 5.11 (s, 2H), 4.68-4.48 (m, 2H), 4.35 (app t, J=7.6 Hz, 1H), 4.18-4.10 (m, 1H), 3.99 (d, J=9.2 Hz, 1H), 3.86-3.74 (m, 2H), 3.44 (s, 1H), 3.40-3.16 (m, 2H), 2.87 (s, 3H), 2.49-2.35 (m, 3H), 1.99 (s, 3H), 1.94-1.80 (m, 2H), 1.51-1.44 (m, 3H), 1.44-1.36 (m, 6H)

### Step 5a. Preparation of Linker-4

To a solution of Step 4a ester (170 mg, 0.279 mmol) in EtOH (10 ml) was added Pd/C (10.0%, 33.9 mg, 0.0319 mmol) moistened with a drop of water. The mixture was stirred under hydrogen (50 PSI) overnight, and then filtered through a short pad of celite. Filter cake washed with EtOH (2×5 ml) and filtrate concentrated on vacuo. Residue triturated with ether (5 ml) to afford the title compound as colourless glassy solid (127 mg, 87%).

LCMS: 100% purity by ELSD; RT=1.18 min; m/z calcd for C₂₁H₃₃N₃O₁₂ [M+H]⁺ 520, found 520; ¹H NMR (400 MHz, MeOD-d4) δ 5.81-5.74 (m, 2H), 5.38 (s, 1H), 4.55 (d, J=5.2 Hz, 1H), 4.52-4.42 (m, 1H), 4.25-4.15 (m, 2H), 3.90 (s, 1H), 3.78-3.72 (m, 1H), 3.69 (s, 1H), 3.61 (dt, J=6.0 Hz, 1H), 3.40 (s, 1H), 3.38-3.25 (m, obscured by solvent residual peak), 2.92 (s, 3H), 2.29 (dt, J=7.6 Hz, 2H), 1.97 (s, 3H), 1.90-1.76 (m, 2H), 1.44 (d, J=7.2 Hz, 3H), 1.35 (d, J=6.8 Hz, 3H)

### Step 5b. Preparation of Linker-5

To a solution of Step 4b ester (110 mg, 0.176 mmol) in EtOH (10 ml) was added Pd/C (10.0%, 33.9 mg, 0.0319 mmol) moistened with a drop of water. The mixture was stirred under hydrogen (50 PSI) overnight, and then filtered through a short pad of celite. Filter cake washed with EtOH (2×5 ml) and filtrate concentrated on vacuo. Residue triturated with ether (5 ml) to afford the title compound as colourless glassy solid (90 mg, 95%).

LCMS: 100% purity by ELSD; RT=1.22 min; m/z calcd for C₂₂H₃₅N₃O₁₂ [M+H]⁺ 534, found 534; ¹H NMR (400 MHz, MeOD-d4) δ 6.82-6.70 (m, 1H), 5.39 (s, 1H), 4.57 (d, J=5.6 Hz, 1H), 4.56-4.36 (m, 1H), 4.25 (d, J=7.2 Hz, 1H), 4.19 (dt, J=6.4 Hz, 1H), 3.89 (s, 1H), 3.80-3.72 (m, 1H), 3.69 (s, 1H), 3.40 (d, J=1.6 Hz, 1H), 3.38-3.23 (m, obscured by solvent residual peak), 2.94-2.86 (m, 3H), 2.30-2.25 (m, 2H), 1.97 (s, 3H), 1.83 (qd, J=6.8 Hz, 2H), 1.50 (dd, J=5.6 Hz, 3H), 1.42 (dd, J=7.2 Hz and 3.2 Hz, 3H), 1.38-1.28 (m, 3H)

### (d) Synthesis of Linker-6

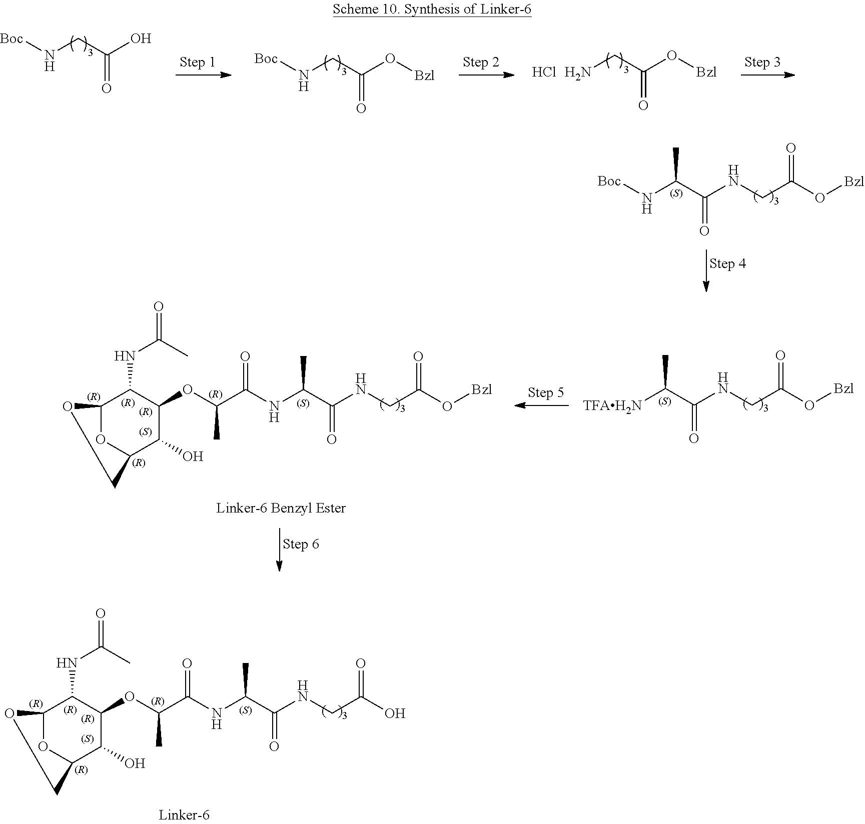

### Step 1. Preparation of Benzyl 4-((tert-butoxycarbonyl)amino)butanoate

To a suspension of 4-(tert-butoxycarbonylamino)butanoic acid (5.00 g, 24.6 mmol) in DMF (50 ml) was added K₂CO₃ (8.84 g, 64.0 mmol) followed by benzylbromide (5.47 g, 32.0 mmol). The reaction was stirred at RT for 4 days. The mixture was poured into cold water (1.5 L) and extracted with diethyl ether (2×300 ml). The extracts were washed with brine and concentrated in vacuo to afford a colourless oil that solidified on standing. This was triturated with hexane and the title compound was isolated by filtration as a white solid (6.93 g, 96%).

LCMS: 96.5% purity; RT=1.76 min; m/z calcd for C₁₆H₂₃NO₄ [M+H-Boc]⁺ 194; ¹H NMR (400 MHz, CDCl3) δ 7.40-7.31 (m, 5H), 5.12 (s, 2H), 4.59 (br s, 1H), 3.23-3.07 (m, 2H), 2.40 (app t, J=7.2 Hz, 2H), 1.83 (app q, J=6.8 Hz, 2H), 1.43 (s, 9H)

### Step 2. Preparation of Benzyl 4-aminobutanoate. hydrochloride

To a stirred solution of Step 1 ester (1.00 g, 3.41 mmol) in dioxane (30 ml) was added HCl (4M in dioxane) (4.00 M, 12.8 mL, 51.1 mmol). The resulting solution was stirred for 3 h. The solvent was removed in vacuo and the residue co-evaporated with dioxane (2×20 ml) to remove HCl traces. Triturated with ether and filtered to afford the title compound as white powder (0.57 g, 72%).

LCMS: 99.0% purity; RT=1.05 min; m/z calcd for free base C₁₁H₁₅NO₂ [M+H]⁺ 194; ¹H NMR (400 MHz, DMSO-d6) δ 8.02 (br s, 3H), 7.40-7.31 (m, 5H), 5.12 (s, 2H), 2.81 (app t, J=7.6 Hz, 2H), 2.55-2.45 (m, obscured by solvent water peak), 1.85 (app q, J=7.6 Hz, 2H)

### Step 3. Preparation of Benzyl (S)-4-(2-((tert-butoxycarbonyl)amino)propanamido)butanoate

Step 2 HCl salt (0.500 g, 2.18 mmol) and N-Boc L-Alanine-OH (0.487 g, 2.57 mmol) were dissolved in DMF (15 ml, 0.1 M) at room temperature. DIEA (1.25 mL, 7.18 mmol) and HATU (0.910 g, 2.39 mmol) were then added and the mixture was stirred for 18 hours at RT. The reaction mixture was added to ice water (250 ml) and the resulting white precipitate was extracted with 20% EtOAc in ether (3×300 ml). The combined extracts were washed with brine (2×300 ml) and dried over sodium sulphate, filtered, and concentrated in vacuo to afford a colourless oil. This was purified by flash column chromatography using a Biotage Flashmaster system (eluent: 10% to 90% EtOAc in hexane gradient). Pure fractions by TLC were combined and concentrated in vacuo to afford the title compound as colourless viscous oil (0.541 g, 68.2%).

LCMS: 100% purity; RT=1.62 min; m/z calcd for C₁₉H₂₈N₂O₅ [M+H]⁺ 365; ¹H NMR (400 MHz, CDCl3) δ 7.38-7.29 (m, 5H), 6.31 (br s, 1H), 5.11 (s, 1H), 4.92 (br s, 1H), 4.13-4.07 (m, 1H), 3.32-3.27 (m, 2H), 2.40 (app t, J=7.2 Hz, 2H), 1.85 (app q, J=7.2 Hz, 2H),

### Step 4. Preparation of Benzyl (S)-4-(2-aminopropanamido)butanoate.trifluoroacetate

To a solution of Step 3 ester (0.540 g, 1.48 mmol) in DCM (10 ml) was added TFA (1.10 mL, 14.8 mmol) in one portion at RT. The solution was stirred for 20 hours at RT. The solvent was removed in vacuo and the residue co-evaporated with toluene (10 ml×3) to remove residual TFA. The title compound was isolated as colourless oil (0.75 g, 134%). The material was used directly in next step.

LCMS: 96.4% purity; RT=1.12 min; m/z calcd for free base C₁₄H₂₀N₂O₃ [M+H]⁺ 265.

### Step 5. Preparation of Benzyl 4-((S)-2-((R)-2-(((1R,2S,3R,4R,5R)-4-acetamido-2-hydroxy-6,8-dioxabicyclo[3.2.1]octan-3-yl)oxy)propanamido)propanamido)butanoate

Step 4 salt (366 mg, 0.966 mmol) and Part 1 Step 3 acid (1,6-Anhydro-N-acetylmuramic acid) (266 mg, 0.966 mmol) were dissolved in DMF (10 ml, ca. 0.1 M) at room temperature. DIEA (0.673 mL, 3.87 mmol) and HATU (404 mg, 1.06 mmol) were then added and the mixture was stirred for 18 hours at RT. The reaction mixture was added to ice water (300 ml) and extracted with EtOAc (3×100 ml), then with 1:1 chloroform-IPA (3×80 ml). Organic fractions combined, dried over sodium sulphate and concentrated in vacuo. The crude product was purified by flash column chromatography on silica (eluent: 5-10% MeOH in DCM gradient). Pure fractions by TLC were combined and concentrated in vacuo. Fractions contaminated with DMF were combined and concentrated in vacuo. The residue was taken up in EtOAc (200 ml) and washed with 5% LiCl aq solution (2×50 ml). Organic solution dried over sodium sulphate and concentrated in vacuo. The crude product was purified by flash column chromatography on silica (eluent: 5-10% MeOH in DCM gradient). Pure fractions by TLC were combined with the first pure batch to afford the title compound as a colourless viscous oil that slowly solidified to a white crystalline solid on standing (387 mg, 76%).

LCMS: 100% purity; RT=1.36 min; m/z calcd for C₂₅H₃₅N₃O₉ [M+H]⁺ 522; ¹H NMR (400 MHz, CDCl3) δ 7.76 (d, J=7.2 Hz, 1H), 7.35-7.31 (m, 5H), 6.61 (app t, J=5.6 Hz, 1H), 6.25 (d, J=9.2 Hz, 1H), 5.41 (s, 1H), 5.10 (s, 2H), 4.52 (d, J=5.2 Hz, 1H), 4.39-4.36 (m, 1H), 4.23 (d, J=7.6 Hz, 1H), 4.18-4.13 (m, 1H), 3.98 (d, J=9.2 Hz, 1H), 3.82-3.78 (m, 1H), 3.73 (s, 1H), 3.43 (s, 1H), 3.37-3.17 (m, 2H), 2.38 (app t, J=7.6 Hz, 2H), 1.98 (s, 3H), 1.84 (app q, J=6.8 Hz, 2H), 1.38-1.35 (m, 6H)

### Step 6. Preparation of Linker-6

To a solution of Step 5 ester (0.300 g, 0.575 mmol) in EtOH (10 ml) was added Pd/C (10.0%, 0.0612 g, 0.0575 mmol) moistened with a drop of water. The mixture was stirred under hydrogen (50 PSI) overnight, and then filtered through a short pad of celite. Filter cake washed with EtOH (2×5 ml) and filtrate concentrated on vacuo. Residue triturated with ether (5 ml) to afford the title compound as off white foamy solid (180 mg, 72%).

LCMS: 100% purity; RT=1.00 min; m/z calcd for C₁₈H₂₉N₃O₉ [M+H]⁺ 432; ¹H NMR (400 MHz, MeOD-d6) δ 5.38 (s, 1H), 4.55 (d, J=5.6 Hz, 1H), 4.37-4.34 (m, 1H), 4.29 (d, J=7.6 Hz, 1H), 4.21-4.16 (m, 1H), 3.87 (s, 1H), 3.76 (dd, J=7.6 and 6.0 Hz, 1H), 3.40 (m, 1H), 3.25-2.22 (m, 2H), 2.32 (app t, J=7.2 Hz, 2H), 1.97 (s, 3H), 1.78 (app q, J=7.2 Hz, 2H), 1.38-1.35 (m, 6H)

### Part 4. Preparation of “SUGAR-LINKER-SPACER-ANTISENSE AGENTS”

Following the general principles detailed in Scheme 3, PMO antisense sequences were coupled with ‘Linkers-1→6’ to provide a range of full ‘constructs’. The PMO antisense agents were purchased pre-prepared from GeneTools Inc (see https://www.gene-tools.com/) with the 5′ end as detailed below and the 3′ end as the free morpholino for attachment of the various linkers.

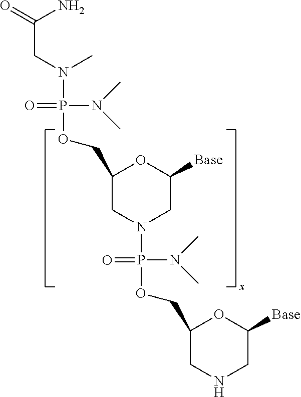

The following ten PMOs based upon the associated Sequence IDs are referred to herein by their PED number as follows:

TABLE 2
PMO, Sequence ID and PED designations

| PED Number | Sequence (5′→3′) | Sequence ID |
|---|---|---|
| PED-1 | CTT CGA TAG TG | 57 |
| PED-2 | TCA AAT GAG GC | 91 |
| PED-3 | CGC TCA TCT AA | 94 |
| PED-4 | ATT GAG TGT CAT | 170 |
| PED-5 | TGC CAT CTT GTT | 171 |
| PED-6 | GTT GTT TGA TC | 102 |
| PED-7 | CAG GAG CAT TAG | 172 |
| PED-8 | GTC TAT TCT CC | 153 |
| PED-9 | CCT CAG ACT CC | 87 |
| PED-10 | AGT GCT CTA CC | 103 |

(a) Preparation of Linkers-1→6 coupled with PMO PED-1.

### Preparation of L1-PED-1 (PED-006)

To a solution of Linker-1 (Part 3(a) Step 5 acid) (2.32 mg, 4.36 μmol) in DMSO (127 μL) was successively added HBTU (0.300 M in DMSO, 15.9 μL, 4.76 μmol), HOAt (0.300 M in DMSO, 15.9 μL, 4.76 μmol) in and DIEA (0.600 M in DMSO, 16.5 μL, 9.91 μmol). The mixture was then immediately added to PED-1 (0.01 M in DMSO, 396 μL, 3.96 μmol). The vial containing the activated acid was washed with DMSO (2×50 μL) and the washings added to the reaction mixture. The mixture was stirred for 2 hours at 40° C. The crude mixture was analysed by LCMS—desired product was observed. The reaction solution was stored overnight at −25 C and purified by preparative HPLC in two equal injections. This gave two product fractions of ca. 10 ml. each. These were combined in a 28 ml glass vial and lyophilised to afford title construct as a white solid (9.1 mg, 53%)

LCMS purity (225 nm+/−50)=100%, RT=1.109 min; m/z calcd for [M]³⁺ 1433.5, found 1433.2.

### Preparation of L2-PED-1 (PED-007)

To a solution of Linker-2 (Part 3(b) Step 5a acid) (2.14 mg, 4.36 μmol) in DMSO (127 μL) was successively added HBTU (0.300 M in DMSO, 15.9 μL, 4.76 μmol), HOAt (0.300 M in DMSO, 15.9 μL, 4.76 μmol) in and DIEA (0.600 M in DMSO, 16.5 μL, 9.91 μmol). The mixture was then immediately added to PED-1 (0.01 M in DMSO, 396 μL, 3.96 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections. This gave two product fractions of ca. 10 ml. each. These were combined in a 28 ml glass vial and lyophilised to afford title construct as a white lyophilised solid (7.4 mg, 43%).

LCMS purity (225 nm+/−50)=98.8%, RT=1.100 min; m/z calcd for [M]³⁺ 1420.1, found 1419.9.

### Preparation of L3-PED-1 (PED-008)

To a solution of Linker-3 (Part 3(b) Step 5b acid) (2.20 mg, 4.36 μmol) in DMSO (127 μL) was successively added HBTU (0.300 M in DMSO, 15.9 μL, 4.76 μmol), HOAt (0.300 M in DMSO, 15.9 μL, 4.76 μmol) in and DIEA (0.600 M in DMSO, 16.5 μL, 9.91 μmol). The mixture was then immediately added to PED-1 (0.01 M in DMSO, 396 μL, 3.96 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections.

This gave two product fractions of ca. 10 ml. each. These were combined in a 28 ml glass vial and lyophilised to afford title construct as a white lyophilised solid (8.5 mg, 50%).

LCMS purity (225 nm+/−50)=96.6%; RT=1.109 min; m/z calcd for [M]³⁺ 1424.8, found 1424.5.

### Preparation of L4-PED-1 (PED-009)

To a solution of Linker-4 (Part 3(c) Step 5a acid) (2.27 mg, 4.36 μmol) in DMSO (127 μL) was successively added HBTU (0.300 M in DMSO, 15.9 μL, 4.76 μmol), HOAt (0.300 M in DMSO, 15.9 μL, 4.76 μmol) in and DIEA (0.600 M in DMSO, 16.5 μL, 9.91 μmol). The mixture was then immediately added to PED-1 (0.01 M in DMSO, 396 μL, 3.96 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections. This gave two product fractions of ca. 10 ml. each. These were combined in a 28 ml glass vial and lyophilised to afford title construct as a white lyophilised solid (10.7 mg, 63%).

LCMS purity (225 nm+/−50)=98.5%, RT=1.111 min; m/z calcd for [M]³⁺ 1429.5, found 1429.3.

### Preparation of L5-PED-1 (PED-010)

To a solution of Linker-5 (Part 3(c) Step 5b acid) (2.33 mg, 4.36 μmol) in DMSO (127 μL) was successively added HBTU (0.300 M in DMSO, 15.9 μL, 4.76 μmol), HOAt (0.300 M in DMSO, 15.9 μL, 4.76 μmol) in and DIEA (0.600 M in DMSO, 16.5 μL, 9.91 μmol). The mixture was then immediately added to PED-1 (0.01 M in DMSO, 396 μL, 3.96 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections. This gave two product fractions of ca. 10 ml. each. These were combined in a 28 ml glass vial and lyophilised to afford title construct as a white lyophilised solid (11.2 mg, 65%).

LCMS purity (225 nm+/−50)=100%, RT=1.119 min; m/z calcd for [M]³⁺ 1434.2, found 1434.0.

### Preparation of L6-PED-1 (PED-011)

To Linker-6 (Part 3(d) Step 6 acid) (solution in anhydrous DMSO) (0.0350 M, 109 μL, 3.81 μmol) was successively added HBTU (solution in DMSO) (0.300 M, 13.8 μL, 4.15 μmol), HOAt (solution in DMSO) (0.300 M, 13.8 μL, 4.15 μmol) and DIEA (solution in DMSO) (0.300 M, 28.8 μL, 8.65 μmol). The mixture was then immediately added to PED-1 oligonucleotide (solution in DMSO) (0.0100 M, 346 μL, 3.46 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections. This gave two product fractions of ca. 10 ml. each. These were combined in a 28 ml glass vial and lyophilised to afford title construct as a white solid (11.9 mg, 2.84 μmol, yield: 81.9%).

LCMS purity (225 nm+/−50)=98.3%, RT=1.114 min; m/z calcd for [M]³⁺ 1400.1, found 1399.9.

### Preparation of L6-PED-2 (PED-012)

To Linker-6 (Part 3(d) Step 6 acid) (solution in anhydrous DMSO) (0.0350 M, 106 μL, 3.70 μmol) was successively added HBTU (0.300 M in DMSO, 13.5 μL, 4.04 μmol), HOAt (0.300 M in DMSO, 13.5 μL 4.04 μmol) and DIEA (0.300 M in DMSO, 28.1 μL, 8.42 μmol). The mixture was then immediately added to PED-2 oligonucleotide (0.0100 M in DMSO, 337 μL, 3.37 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections. This gave two product fractions of ca. 10 ml. each. These were combined in a 28 ml glass vial and lyophilised to afford title construct as a white solid (9.8 mg, 69%).

LCMS purity (225 nm+/−50)=97.3%, RT=1.117 min; m/z calcd for [M]³⁺ 1406.1, found 1405.9.

### Preparation of L6-PED-3 (PED-013)

To Linker-6 (Part 3(d) Step 6 acid) (solution in anhydrous DMSO) (0.0350 M, 79.6 μL, 2.78 μmol) was successively added HBTU (solution in DMSO) (0.300 M, 10.1 μL, 3.04 μmol), HOAt (solution in DMSO) (0.300 M, 10.1 μL, 3.04 μmol) and DIEA (solution in DMSO) (0.300 M, 21.1 μL, 6.33 μmol). The mixture was then immediately added to PED-3 oligonucleotide (solution in DMSO) (0.0100 M, 253 μL, 2.53 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections. This gave two product fractions of ca. 10 ml. each. These were combined in a 28 ml glass vial and lyophilised to afford title construct as a white solid (9.30 mg, 2.25 μmol, yield: 89.0%).

LCMS purity (225 nm+/−50)=96.6%, RT=1.107 min; m/z calcd for [M]³⁺ 1376.5, found 1376.2.

### Preparation of L6-PED-4 (PED-014)

To Linker-6 (Part 3(d) Step 6 acid) (solution in anhydrous DMSO) (0.0350 M, 86.7 μL, 3.03 μmol) was successively added HBTU (solution in DMSO) (0.300 M, 11.0 μL, 3.31 μmol), HOAt (solution in DMSO) (0.300 M, 11.0 μL, 3.31 μmol) and DIEA (solution in DMSO) (0.300 M, 23.0 μL, 6.89 μmol). The mixture was then immediately added to PED-4 oligonucleotide (solution in DMSO) (0.0100 M, 276 μL, 2.76 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections. This gave two product fractions of ca. 10 ml. each. These were combined in a 28 ml glass vial and lyophilised to afford title construct as a white solid (8.00 mg, 1.76 μmol, yield: 63.7%).

LCMS purity (225 nm+/−50)=96.5%, RT=1.155 min; m/z calcd for [M]⁴⁺ 1138.9, found 1139.9.

### Preparation of L6-PED-5 (PED-015)

To Linker-6 (Part 3(d) Step 6 acid) (solution in anhydrous DMSO) (0.0350 M, 95.9 μL, 3.36 μmol) was successively added HBTU (solution in DMSO) (0.300 M, 12.2 μL, 3.66 μmol), HOAt (solution in DMSO) (0.300 M, 12.2 μL, 3.66 μmol) and DIEA (solution in DMSO) (0.300 M, 25.4 μL, 7.63 μmol). The mixture was then immediately added to PED-5 oligonucleotide (solution in DMSO) (0.0100 M, 305 μL, 3.05 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections. This gave two product fractions of ca. 10 ml. each. These were combined in a 28 ml glass vial and lyophilised to afford title construct as a white solid (8.70 mg, 1.94 μmol, yield: 63.7%).

LCMS purity (225 nm+/−50)=100%, RT=1.152 min; m/z calcd for [M]³⁺ 1493.9, found 1493.8.

### Preparation of L6-PED-6 (PED-016)

To Linker-6 (Part 3(d) Step 6 acid) (solution in anhydrous DMSO) (0.0350 M, 78.8 μL, 2.76 μmol) was successively added HBTU (solution in DMSO) (0.300 M, 10.0 μL, 3.01 μmol) HOAt (solution in DMSO) (0.300 M, 10.0 μL, 3.01 μmol) and DIEA (solution in DMSO) (0.300 M, 20.9 μL, 6.27 μmol). The mixture was then immediately added to PED-6 oligonucleotide (solution in DMSO) (0.0100 M, 251 μL, 2.51 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections. This gave two product fractions of ca. 10 ml. each. These were combined in a 28 ml glass vial and lyophilised to afford title construct as a white solid (6.80 mg, 1.62 μmol, yield: 64.5%).

LCMS purity (225 nm+/−50)=95.4%, RT=1.163 min; m/z calcd for [M]³⁺ 1402.1, found 1402.0.

### Preparation of L6-PED-7 (PED-017)

To Linker-6 (Part 3(d) Step 6 acid) (solution in anhydrous DMSO) (0.0350 M, 88.4 μL, 3.10 μmol) was successively added HBTU (solution in DMSO) (0.300 M, 11.3 μL, 3.38 μmol), HOAt (solution in DMSO) (0.300 M, 11.3 μL, 3.38 μmol) and DIEA (solution in DMSO) (0.300 M, 23.5 μL, 7.04 μmol). The mixture was then immediately added to PED-7 oligonucleotide (solution in DMSO) (0.0100 M, 281 μL, 2.81 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections. This gave two product fractions of ca. 10 ml. each. These were combined in a 28 ml glass and lyophilised to afford title construct as a white solid (7.00 mg, 1.53 μmol, yield: 54.4%).

LCMS purity (225 nm+/−50)=97.5%, RT=1.104 min; m/z calcd for [M]⁴⁺ 1143.7, found 1143.5.

### Preparation of L6-PED-8 (PED-018)

To Linker-6 (Part 3(d) Step 6 acid) (solution in anhydrous DMSO) (0.0350 M, 94.4 μL, 3.30 μmol) was successively added HBTU (solution in DMSO) (0.300 M, 12.0 μL, 3.60 μmol), HOAt (solution in DMSO) (0.300 M, 12.0 μL, 3.60 μmol) and DIEA (solution in DMSO) (0.300 M, 25.0 μL, 7.51 μmol). The mixture was then immediately added to PED-8 oligonucleotide (solution in DMSO) (0.0100 M, 300 μL, 3.00 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections. This gave two product fractions of ca. 10 ml. each. These were combined in a 28 ml glass vial and lyophilised to afford title construct as a white solid (9.30 mg, 2.26 μmol, yield: 75.4%).

LCMS purity (225 nm+/−50)=95.3%, RT=1.112 min; m/z calcd for [M]³⁺ 1370.4, found 1370.3.

### Preparation of L6-PED-9 (PED-019)

To Linker-6 (Part 3(d) Step 6 acid) (solution in anhydrous DMSO) (0.0350 M, 93.2 μL, 3.26 μmol) was successively added HBTU (solution in DMSO) (0.300 M, 11.9 μL, 3.56 μmol), HOAt (solution in DMSO) (0.300 M, 11.9 μL, 3.56 μmol) and DIEA (solution in DMSO) (0.300 M, 24.7 μL, 7.42 μmol). The mixture was then immediately added to PED-9 oligonucleotide (solution in DMSO) (0.0100 M, 297 μL, 2.97 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections. This gave two product fractions of ca. 10 ml. each. These were combined in a 28 ml glass vial and lyophilised to afford title construct as a white solid (8.20 mg, 2.01 μmol, yield: 67.6%).

LCMS purity (225 nm+/−50)=97%, RT=1.080 min; m/z calcd for [M]³⁺ 1363.4, found 1363.4.

### Preparation of L6-PED-10 (PED-020)

To Linker-6 (Part 3(d) Step 6 acid) (solution in anhydrous DMSO) (0.0350 M, 87.7 μL, 3.07 μmol) was successively added HBTU (solution in DMSO) (0.300 M, 11.2 μL, 3.35 μmol), HOAt (solution in DMSO) (0.300 M, 11.2 μL, 3.35 μmol) and DIEA (solution in DMSO) (0.300 M, 23.2 μL, 6.97 μmol). The mixture was then immediately added to PED-10 oligonucleotide (solution in DMSO) (0.0100 M, 279 μL, 2.79 μmol). The vial containing the activated acid was washed with DMSO (2×50 μL) and the washings added to the reaction mixture. The mixture was stirred for 2 hours at 40° C. The crude mixture was analysed by LCMS—desired product is observed. The reaction solution was stored overnight at −25 C and purified by preparative HPLC in two equal injections. This gave two product fractions of ca. 10 ml. each. These were combined in a 28 ml glass vial and lyophilised to afford title construct as a white solid (8.20 mg, 1.98 μmol, yield: 71.0%).

LCMS purity (225 nm+/−50)=100%. RT=1.100 min; m/z calcd for [M]³⁺ 1381.8, found 1381.7

Stability Testing Procedures

(i) Plasma Stability (Human, Mouse and/or Rat)

To quantify the degradation of the test compound in plasma over a 1 hour period. The percent of parent compound present at 0, 30 and 60 mins after initiating incubations in plasma is determined. Compounds are taken from 10 mM DMSO stock solutions and added to plasma, which has previously been incubated at 37° C., to give a final concentration of 25 μM and re-incubated. Aliquots are removed at the appropriate timepoints and quenched with an equal volume of cold acetonitrile. After mixing vigorously, the precipitated protein matter are removed by filtration (Multiscreen Solvinert filter plates, Millipore, Bedford, Mass., USA) and the filtrate analysed by reverse phase HPLC with mass spectrometric detection, using single ion monitoring of the [M+H]⁺ species. Metabolic turnover is determined by comparison of peak areas from the ion chromatograms of the parent before and after incubation and expressed as percent remaining at each timepoint.

Plasma stability data for the six full conjugates derived from PED-1 and the Linkers-1-6 is detailed in Table 3.

TABLE 3
Plasma stability for different linker constructs

| | Gram | Antisense | | | | |
|---|---|---|---|---|---|---|
| | Negative | Oligonucleotide | | | | Plasma Stability |
| Target | Organism | Sequence | Compound | Sugar-Spacer-Linker | Species | Half-life (min) |
| AcpP | E.Coli | CTT CGA TAG TG | PED-006 (L1) | Sugar-L- Ala-OCH₂O-PIP-OH | Mouse | 161 |
| | | (PED-1) | | | Human | 133 |
| | | | PED-007 (L2) | Sugar-L-Ala-OCH₂O-N-Me-Gly-OH | Mouse | 59 |
| | | | | | Human | 77 |
| | | | PED-008 (L3) | Sugar-L-Ala-OCH(Me)O-N-Me-Gly-OH | Mouse | 136 |
| | | | | | Human | 83 |
| | | | PED-009 (L4) | Sugar-L-Ala-OCH₂O-N-Me(CH₂)₃COOH | Mouse | 6 |
| | | | | | Human | 47 |
| | | | PED-010 (L5) | Sugar-L-Ala-OCH(Me)O-N-Me(CH₂)₃COOH | Mouse | 26 |
| | | | | | Human | 101 |
| | | | PED-011 (L6) | Sugar-L-Ala-NH(CH₂)₃COOH | Mouse | 262 |
| | | | | | Human | 263 |

(ii) Microsomal Metabolic Stability (Human, Mouse and/or Rat)

Test compound (3 μM) is incubated with pooled liver microsomes. Test compound is incubated at 5 time points over the course of a 45 min experiment and the test compound is analysed by LC-MS/MS. An intrinsic clearance value (CLᵢₙₜ) with standard error and t % value are calculated.

Microsomes (final protein concentration 0.5 mg/mL), 0.1M phosphate buffer pH7.4 and test compound (final substrate concentration 3 μM; final DMSO concentration 0.25%) are pre-incubated at 37 C prior to the addition of NADPH (final concentration 1 mM) to initiate the reaction. The final incubation volume is 50 μL. A minus cofactor control incubation is included for each compound tested where 0.1 M phosphate buffer pH7.4 is added instead of NADPH (minus NADPH). Two control compounds are included with each species. All incubations are performed singularly for each test compound. Each compound is incubated for 0, 5, 15, 30 and 45 min. The control (minus NADPH) is incubated for 45 min only. The reactions are stopped by transferring 20 μL of incubate to 60 μL methanol at the appropriate time points. The termination plates are centrifuged at 2,500 rpm for 20 min at 4 C to precipitate the protein. Following protein precipitation, the sample supernatants are combined in cassettes of up to 4 compounds and analysed using generic LC-MS/MS conditions. From a plot of ln peak area ratio (compound peak area/internal standard peak area) against time, the gradient of the line is determined. Subsequently, half-life and intrinsic clearance are calculated using the equations below:

$$Elimination rate constant ( k ) = ( - gradient )$$

$$Half \text{-} life ( {t}_{1 / 2} ) ( min ) = \frac{0.693}{k}$$

$$Intrinsic clearance ( C {L}_{i n t} ) ( µL \text{/} min \text{/} mg protein ) = \frac{V \times 0.693}{{t}_{1 / 2}}$$

where V=Incubation volume (μL)/Microsomal protein (mg)

Relevant control compounds are assessed, ensuring intrinsic clearance values fall within the specified limits.

(iii) Hepatocyte Stability (Human, Mouse and/or Dog)

Test compound (3 μM) is incubated with cryopreserved hepatocytes in suspension. Samples are removed at 6 time points over the course of a 60 min experiment and test compound is analysed by LC-MS/MS. An intrinsic clearance value (CLᵢₙₜ) with standard error and half-life (t_{1/2}) are calculated. Cryopreserved pooled hepatocytes are stored in liquid nitrogen prior to use. Williams E media supplemented with 2 mM L-glutamine and 25 mM HEPES and test compound (final substrate concentration 3 μM; final DMSO concentration 0.25%) are pre-incubated at 37 C prior to the addition of a suspension of cryopreserved hepatocytes (final cell density 0.5×10⁶ viable cells/mL in Williams E media supplemented with 2 mM L-glutamine and 25 mM HEPES) to initiate the reaction. The final incubation volume is 500 μL. A control incubation is included for each compound tested where lysed cells are added instead of viable cells. Two control compounds are included with each species.

The reactions are stopped by transferring 50 μL of incubate to 100 μL methanol containing internal standard at the appropriate time points. The control (lysed cells) is incubated for 60 min only. The termination plates are centrifuged at 2500 rpm at 4° C. for 30 min to precipitate the protein. Following protein precipitation, the sample supernatants are combined in cassettes of up to 4 compounds and analysed using generic LC-MS/MS conditions. From a plot of ln peak area ratio (compound peak area/internal standard peak area) against time, the gradient of the line is determined. Subsequently, half-life (t_{1/2}) and intrinsic clearance (CLᵢₙₜ) are calculated using the equations below:

$$Elimination rate constant ( k ) = ( - gradient )$$

$$Half \text{-} life ( {t}_{1 / 2} ) ( min ) = \frac{0.693}{k}$$

$$Intrinsic clearance ( C {L}_{i n t} ) ( µL \text{/} min \text{/} million cells ) = \frac{V \times 0.693}{{t}_{1 / 2}}$$

where V=Incubation volume (μL)/Number of cells

Two control compounds for each species are included in the assay and if the values for these compounds are not within the specified limits the results are rejected and the experiment repeated.
Result; The construct L6-PED-1 (PED-011) showed human, mouse & dog hepatocyte stability all with low Clint <10 mL/min/10⁶ cells

(iv) Whole Human Blood Stability (Human, Mouse and/or Rat)

The test compound is incubated with fresh human (mixed sex) blood at 37 C at 5 time points over a 60 min period. The samples are analysed by LC-MS/MS and the percent of parent compound remaining is calculated for each time-point. The percent parent compound remaining at each time point is determined.

Fresh human (mixed sex) blood is used. Single incubations are performed at a test or control compound concentration of 1 μM in blood at 37° C. The final DMSO concentration in the incubation is 0.25%. A control compound is included with each species. Reactions are terminated following 0, 5, 15, 30 and 60 min by acetonitrile containing internal standard. The sampling plate is centrifuged (3000 rpm, 45 min, 4 C) and the supernatants from each time point analysed for parent compound by LC-MS/MS. The percentage of parent compound remaining at each time point relative to the 0 min sample is then calculated from LC-MS/MS peak area ratios (compound peak area/internal standard peak area).

(v) Log D Determinations:

Log D_{(PBS)} determinations is performed in 96 well microtitre plates using a miniaturised “shake-flask” method. In brief, compounds are taken from 10 mM DMSO stock solutions and added to wells containing equal volumes of phosphate buffered saline (10 mM; pH 7.4) (PBS) and 1-octanol (Sigma-Aldrich, Poole, Dorset, UK) to give a final concentration of 50 μM. The plates are then capped and mixed vigorously for 1 hour on a microtitre plate shaker, after which they were left to stand, allowing the PBS and octanol phases to separate. The PBS layer is analysed by reverse phase HPLC with mass spectrometric detection, using single ion monitoring of the [M+H]⁺ species. Log D_{(PBS)} is determined by comparison of the peak area from the ion chromatogram of the compound in the PBS phase with that of a 50 μM standard of the same compound dissolved in acetonitrile/water (50:50) and calculated using the following formula:

$$Log D = Log [ \frac{A U C s t d - A U C p b s}{A U C p b s} ]$$

Where AUCstd and AUCpbs are the peak areas from the standard and test ion chromatograms respectively. Log D_{(PBS)} determinations were also made using PBS at pH6.9 and 5.5 by adjusting the pH of the buffer prior to the start of the assay, with 0.1 M HCL.
Result; The construct L6-PED-1 (PED-011) Log D_{7.4}=0.74

Determination of Chemical Stability as a Function of PH

The chemical stability of the compounds of the invention is studied as a function of pH vs time. The loss of the compound and formation of released parent is quantified by RP-HPLC as appropriate.

General Procedure for HPLC Stability Tests

All chemical stability tests (0.2-2.0 mg/mL) are performed at 37° C. in duplicate with or without co-solvent (MeCN or DMSO) depending on the solubility in the following pH buffered solutions. The results are presented as the mol % of both the compound and parent antibacterial present initially and at the final time point (measured by HPLC peak integration). In order to calculate the concentration of the parent antibacterial formed, it is necessary to calibrate the HPLC using a pure standard to take into account any difference in the extinction coefficients.

(i) pH 1.2 0.1 M Chloride Buffer

This is prepared by dissolving NaCl (0.2 g) in 90 mL of distilled water and adjusting the pH to 1.2 with approximately 5 mL of 1 M hydrochloric acid. The volume is made up to 100 mL with distilled water and if required, adjusted to pH 1.2 with a few drops of 1 M hydrochloric acid. The test conditions are 37° C. and a total time of 1 hour.

(ii) pH 3.0 0.1 M Citrate Buffer

This is prepared by adding 1 M sodium hydroxide (4-5 mL) to 100 mL of 0.1 M aqueous citric acid until a pH of 3.0 is obtained. The test conditions are 20° C. and a total time of 2 hours.

(iii) pH 6.8 0.1 M Phosphate buffer

This is prepared by adding 1 M sodium hydroxide to 100 mL of 0.1 M aqueous sodium dihydrogen phosphate until a pH of 6.8 is obtained. The test conditions are 37° C. and a total time of 2 hours.

(iv) pH 7.4 0.1 M Phosphate buffer

This is prepared by adding 1 M sodium hydroxide to 100 mL of 0.1 M aqueous sodium dihydrogen phosphate until a pH of 7.4 is obtained. The test conditions are 37° C. and a total time of 2 hours.

(v) pH 8.0 0.1 M Phosphate buffer

This is prepared by adding 1 M sodium hydroxide to 100 mL of 0.1 M aqueous sodium dihydrogen phosphate until a pH of 8.0 is obtained. The test conditions are 20° C. and a total time of 2 hours. Chemical stability data for the six full conjugates derived from PED-1 and the Linkers-1-6 is detailed in Table 4:

TABLE 4
Chemical stability for different linker-constructs

| | Gram | Antisense | | | | |
|---|---|---|---|---|---|---|
| | Negative | Oligonucleotide | | | Buffer | Chemical Stability |
| Target | Organism | Sequence | Compound | Sugar-Spacer-Linker | pH | Half-life (min) |
| AcpP | E.Coli | CTT CGA TAG TG | PED-006 (L1) | Sugar-L- Ala-OCH₂O-PIP-OH | 3.5 | 143 |
| | | (PED-1) | | | 5.0 | 326 |
| | | | | | 7.4 | 276 |
| | | | PED-007 (L2) | Sugar-L-Ala-OCH₂O-N-Me-Gly-OH | 3.0 | 438 |
| | | | | | 5.0 | >480 |
| | | | | | 7.4 | >480 |
| | | | PED-008 (L3) | Sugar-L-Ala-OCH(Me)O-N-Me-Gly-OH | 3.0 | >480 |
| | | | | | 5.0 | 303 |
| | | | | | 7.4 | 275 |
| | | | PED-009 (L4) | Sugar-L-Ala-OCH₂O-N-Me(CH₂)₃COOH | 3.0 | 316 |
| | | | | | 5.0 | 275 |
| | | | | | 7.4 | 241 |
| | | | PED-010 (L5) | Sugar-L-Ala-OCH(Me)O-N-Me(CH₂)₃COOH | 3.0 | 293 |
| | | | | | 5.0 | >480 |
| | | | | | 7.4 | >480 |
| | | | PED-011 (L6) | Sugar-L-Ala-NH(CH₂)₃COOH | 3.0 | 366 |
| | | | | | 5.0 | 159 |
| | | | | | 7.4 | >480 |

In vivo Infection Model Studies

Efficacy of PED-011 Against E. coli (ATCC25922) in the Urinary Tract Infection Mouse Model

Female BALB/c mice were infected trans-ureth rally with ˜ 5×10⁸ CFU/animal E. coli (ATCC 25922). Four hours post infection animals were treated twice daily (Q12 h) orally with ciprofloxacin at 30 mg/kg and with single IV doses of PED-011 at 10 and 30 mg/kg (u.i.d). Ten animals from each group were terminated at 48 h post treatment, bladder and kidneys were collected to determine the bacterial load.

| | | | | Time point | |
|---|---|---|---|---|---|
| | | | Dose | (h) PI | |
| | | | time | for bacterial | |
| | | Treatment | PI | enumeration | No. of |
| Group | Treatment | duration | (h) | in tissues | Animals/group |
| 1 | Early Infection Control | NA | NA | 4 | 10 |
| 2 | Vehicle Control (vehicle, PBS, IV, | 48 h | 4 h | 52* | 10 |
| | Q24 h, 1 doses) | | | | |
| 3 | Ciprofloxacin (30 mg/kg, p.o., b.i.d. | 48 h | 4 h | 52* | 10 |
| | Q12h) | | | | |
| 4 | PED-011 (10 mg/kg, IV, Q24h, 1 | 48 h | 4 h | 52* | 10 |
| | dose) | | | | |
| 5 | PED-011 (30 mg/kg, IV, Q24h, 1 | 48 h | 4 h | 52* | 10 |
| | dose) | | | | |

*48 h post treatment

The vehicle for Ciprofloxacin was 0.25% of Carboxy Methyl Cellulose (CMC) (w/v) and PBS was used for PED-011. PED-011 was dissolved in PBS at 2 & 6 mg/mL. At four hours post infection, animals were treated with the first oral dose of ciprofloxacin and the single dose of PED-011 (10 and 30 mg/kg, IV, single bolus dose, 5 ml/kg dose volume). Animals received further doses of oral ciprofloxacin as per the dosing schedule.

Results

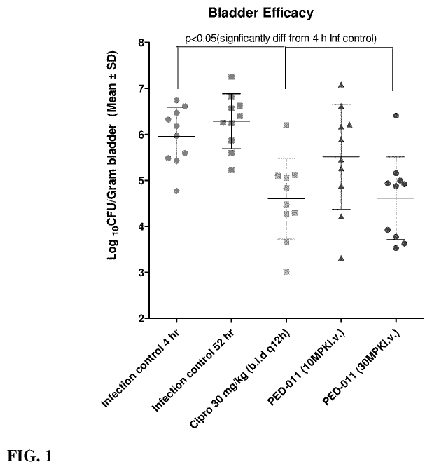

Ciprofloxacin (30 mg/kg, p.o., Q12 h) showed significant antibacterial effect in the bladder and kidneys, when compared to the 4 h control and vehicle control at 48 h post treatment (p<0.05) (FIGS. 1 & 2).

PED-011 (30 mg/kg, IV, single dose) showed significant antibacterial activity in bladder when compared to the 4 h PI control and vehicle control at 48 h post treatment (p<0.05); PED-011 (10 mg/kg, IV, single dose) was not significantly effective when compared to the 4 h and the vehicle control at 48 h post treatment. (FIG. 1)

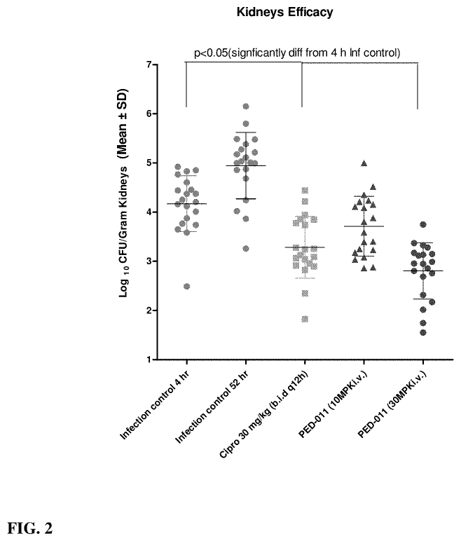

PED-011 (30 mg/kg, IV, single dose) showed significant antibacterial activity in kidneys when compared to the 4 h PI control and vehicle control at 48 h post treatment (p<0.05); PED-011 (10 mg/kg, IV, single dose) showed significant antibacterial activity in kidneys when compared to the 4 h PI control and vehicle control at 48 h post treatment (p<0.05). (FIG. 2)

Efficacy of PED-011 Against E. coli (CFT073, ATCC®700928™) in the Urinary Tract Infection Mouse Model; Challenge with a Uropathogenic Strain

Female BALB/c mice were infected trans-urethrally with ˜5×10⁸ CFU/animal E. coli (CFT073, ATCC®700928™). Twenty-four hours post infection animals were treated twice daily (Q12 h) orally with ciprofloxacin at 30 mg/kg and with single IV doses of PED-011 at 3, 10 and 30 mg/kg (u.i.d). Ten animals from each group were terminated at 48 h post treatment (72 h post infection); bladder and kidneys were collected to determine the bacterial load.

| | | | | Time point | |
|---|---|---|---|---|---|
| | | | Dose | (h) PI | |
| | | | time | for bacterial | |
| | | Treatment | PI | enumeration | No. of |
| Group | Treatment | duration | (h) | in tissues | Animals/group |
| 1 | Early Infection Control | NA | NA | 24 | 10 |
| 2 | Vehicle Control (vehicle, PBS, IV, | 48 h | 24 h | 72* | 10 |
| | Q24 h, 1 doses) | | | | |
| 3 | Ciprofloxacin (30 mg/kg, p.o., b.i.d. | 48 h | 24 h | 72* | 10 |
| | Q12h) | | | | |
| 4 | PED-011 (3 mg/kg, IV, 1 dose) | 48 h | 24 h | 72* | 10 |
| 5 | PED-011 (10 mg/kg, IV, 1 dose) | 48 h | 24 h | 72* | 10 |
| 6 | PED-011 (30 mg/kg IV, 1 dose) | 48 h | 24 h | 72* | 10 |

*48 h post treatment; 72 h post infection

The vehicle for Ciprofloxacin was 0.25% of Carboxy Methyl Cellulose (CMC) (w/v) and PBS was used for PED-011. PED-011 was dissolved in PBS at 0.6, 2 & 6 mg/mL. At twenty-four hours post infection, animals were treated with the first oral dose of ciprofloxacin and the single dose of PED-011 (10 and 30 mg/kg, IV, single bolus dose, 5 ml/kg dose volume). Animals received further doses of oral ciprofloxacin as per the dosing schedule.

Results

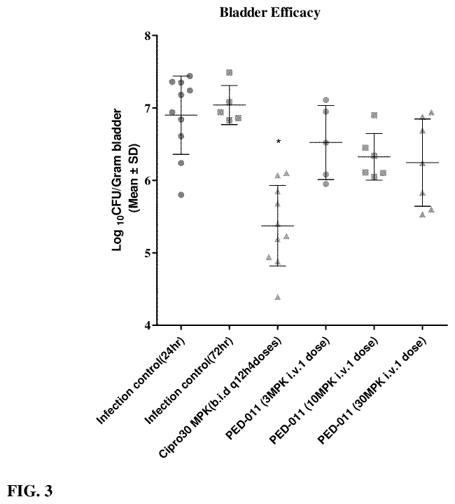

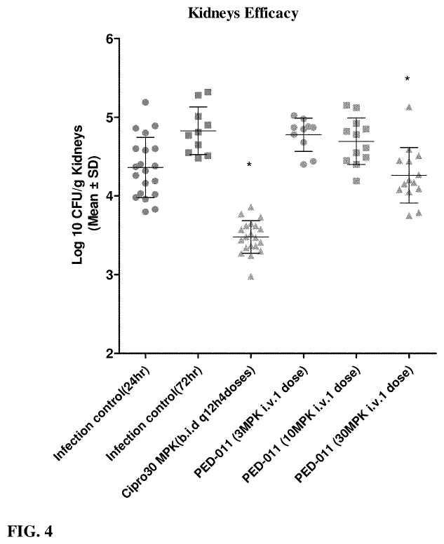

In bladder, Ciprofloxacin showed significant (p<0.05) bactericidal activity at 72 h wrt 24 h PI control and the vehicle control (p<0.05) (FIG. 3). In kidney, Ciprofloxacin showed significant bactericidal activity wrt 24 h PI control and the vehicle control (p<0.05) (FIG. 4).

In bladder, although PED-011 (3, 10 and 30 mg/kg, IV, 1 dose) did not show significant antibacterial effect wrt vehicle control (p>0.05), there appeared to be a dose dependent mean reduction in bacterial counts in bladder (FIG. 3). In kidney, PED-011 (3, 10 and 30 mg/kg, IV, 1 dose) showed dose dependent antibacterial effect with significant effect at 30 mg/kg (p<0.05) when compared to the vehicle control (FIG. 4).

Efficacy of PED-011 Against E. coli (ATCC BAA-2340) in the Urinary Tract Infection Mouse Model; Challenge with a Resistance Strain

Female BALB/c mice were infected trans-urethrally with ˜5×10⁸ CFU/animal E. coli (ATCC BAA-2340). Twenty-four hours post infection animals were treated twice daily (Q12 h) orally with ciprofloxacin at 30 mg/kg and with single IV doses of PED-011 at 10 and 30 mg/kg (u.i.d). Ten animals from each group were terminated at 48 h post treatment; bladder and kidneys were collected to determine the bacterial load.

| | | | | Time point | |
|---|---|---|---|---|---|
| | | | | (h) PI | |
| | | | Dose | for bacterial | |
| | | Treatment | time | enumeration | No. of |
| Group | Treatment | duration | PI (h) | in tissues | Animals/group |
| 1 | Early Infection Control | NA | NA | 24 | 10 |
| 2 | Vehicle Control (vehicle, PBS, IV, | 48 h | 24 h | 72* | 10 |
| | Q24 h, 1 doses) | | | | |
| 3 | Ciprofloxacin (30 mg/kg, p.o., | 48 h | 24 h | 72* | 10 |
| | b.i.d. Q12h) | | | | |
| 4 | PED-011 (10 mg/kg, IV, 1 dose) | 48 h | 24 h | 72* | 10 |
| 5 | PED-011 (30 mg/kg, IV, 1 dose) | 48 h | 24 h | 72* | 10 |

*48 h post treatment

The vehicle for Ciprofloxacin was 0.25% of Carboxy Methyl Cellulose (CMC) (w/v) and PBS was used for PED-011. PED-011 was dissolved in PBS at 2 & 6 mg/mL. At twenty-four hour post infection, animals were be treated with the first oral dose of ciprofloxacin and the single dose of PED-011 (10 and 30 mg/kg, IV, single bolus dose, 5 ml/kg dose volume). Animals received further doses of oral ciprofloxacin as per the dosing schedule.

Results

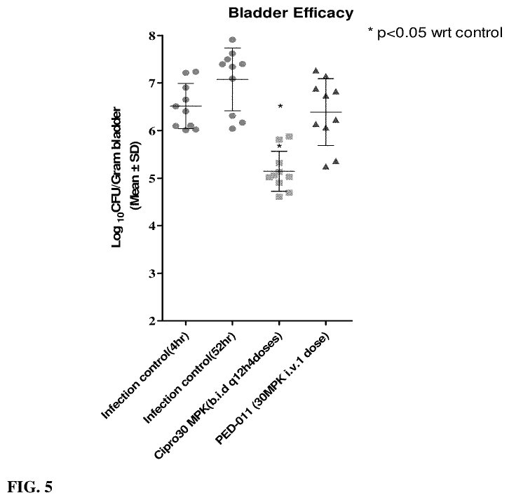

Ciprofloxacin (30 mg/kg, p.o., Q12 h) showed significant antibacterial effect in the bladder and kidneys, when compared to the 24 h control and vehicle control at 48 h post treatment (p<0.05) (FIGS. 5 & 6).

PED-011 (30 mg/kg, IV, single dose) showed a reduction but not statistically significant antibacterial activity in bladder when compared to the 24 h PI control and vehicle control at 48 h post treatment (p<0.05); PED-011 (10 mg/kg, IV, single dose) was not significantly effective when compared to the 4 h and the vehicle control at 48 h post treatment. (FIG. 5)

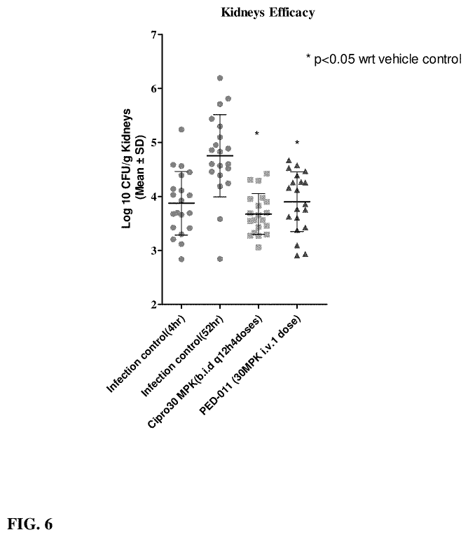

PED-011 (30 mg/kg, IV, single dose) showed significant antibacterial activity in kidneys when compared to the 24 h PI control and vehicle control at 48 h post treatment (p<0.05); PED-011 (10 mg/kg, IV, single dose) showed significant antibacterial activity in kidneys when compared to the 4 h PI control and vehicle control at 48 h post treatment (p<0.05). (FIG. 6)

Efficacy of PED-012 against A. baumannii (ATCC19606) in a Neutropenic Lung Infection Model in Mice

The purpose of this study was to characterize the efficacy of PED-012, following intranasal administration, at 10 and 30 mg/kg dose levels against A. baumannii (ATCC19606) in a neutropenic lung infection model in mice.

Female BALB/c mice were infected by injecting 0.02 ml (containing ˜1×10⁹ CFU/ml) of the inoculum; 10 μl into each nostril of the anesthetized animal intra-nasally using 10 μl pipette (˜2×10⁷ CFU/animal). A gentle mixing of inoculum between two animals will be followed for uniform distribution. Four hours post infection animals were treated twice daily (Q12 h) orally with ciprofloxacin at 30 mg/kg and with single IN doses of PED-012 at 10 and 30 mg/kg (in PBS). Ten animals from each group were terminated at 48 h post treatment; lungs were collected to determine the bacterial load.

| | | | | Time point | |
|---|---|---|---|---|---|
| | | | | (h) PI | |
| | | | Dose | for bacterial | |
| | | Treatment | time | enumeration | No. of |
| Group | Treatment | Duration | PI (h) | in lungs | Animals/group |
| 1 | Early Infection Control | NA | NA | 4 | 5 |
| 2 | Late Infection Control (vehicle, | 48 h | 4 h | 52* | 5 |
| | single dose, IN) | | | | |
| 3 | Ciprofloxacin (10 mg/kg, po, | 48 h | 4 h | 52* | 5 |
| | b.i.d., Q12 h) | | | | |
| 4 | Test compound PED-012 (10 | 48 h | 4 h | 52* | 5 |
| | mg/kg), IN, single dose | | | | |
| 5 | Test compound PED-012 (30 | 48 h | 4 h | 52* | 5 |
| | mg/kg), IN, single dose | | | | |

*48 h post treatment; IN: Intranasal

Results
Ciprofloxacin (10 mg/kg, po, Q12 h) showed significant antibacterial effect when compared to the 52 h vehicle control (p<0.05).
PED-012 [30 mg/kg, IN, single dose] showed significant antibacterial activity when compared to the 52 h vehicle control (p<0.05); PED-012 (10 mg/kg, IN, single dose) was not significantly effective when compared to the vehicle control at 52 h PI (p>0.05).

1. A compound of formula (I), or a pharmaceutically acceptable salt thereof, 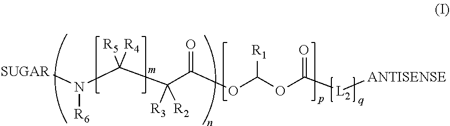
wherein,
ANTISENSE is an oligonucleotide having natural, artificial and/or modified nucleobases, the oligonucleotide selected from the group consisting of phosphodiester oligonucleotides (PDOs), phosphorothioate oligonucleotides (PSOs), phosphorodiamidate morpholino oligonucleotides (PMOs), peptide nucleic acids (PNAs), locked nucleic acids (LNAs), 2′-O-Alkyl oligonucleotides (2′-O-Me, 2′-O-Et. 2′-O-methoxyethyl) and combinations thereof; wherein the oligonucleotide is bonded to the remainder of the molecule of formula I via a terminal amino group present within the ANTISENSE sequence; and
L₂ is a spacer that forms a chemical bond to a terminal amino group present within the ANTISENSE sequence and a second chemical bond to the terminal carbonyl of the remainder of the molecule of formula I and is chosen from the group consisting of: 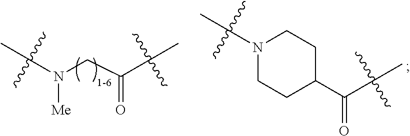
SUGAR is any tautomeric form of the acyl fragment of an N-acylmuramic acid or 1,6-anhydro-N-acylmuramic acid having the structure: 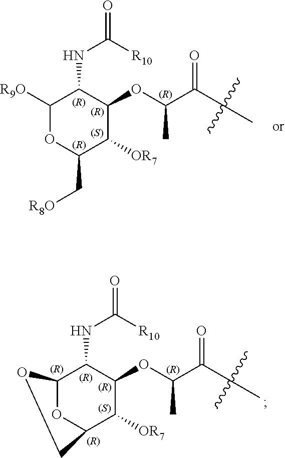
R₁ and R₆ are each independently selected from the group consisting of:
H, C₁₋₆ alkyl, C₁₋₆ substituted alkyl, C₃₋₈ cycloalkyl, C₃₋₈ substituted cycloalkyl, phenyl and benzyl;
R₂ and R₃ are each independently selected from the group consisting of:
H, C₁₋₆ alkyl, C₁₋₆ substituted alkyl, C₃₋₈ cycloalkyl, C₃₋₈ substituted cycloalkyl, phenyl and benzyl, or both together with the carbon atom to which they are attached form a ring containing 3, 4, 5 or 6 carbon atoms; and
R₄ and R₅ are each independently selected from the group consisting of:
H, C₁₋₆ alkyl, C₁₋₆ substituted alkyl, C₃₋₈ cycloalkyl, C₃₋₈ substituted cycloalkyl, phenyl and benzyl, or both together with the carbon atom to which they are attached form a ring containing 3, 4, 5 or 6 carbon atoms;
or
R₂ and R₄ together with the adjacent carbon atoms to which they are attached form a ring containing 3, 4, 5 or 6 carbon atoms; and R₃ and R₅ are each independently selected from the group consisting of: H, C₁₋₆ alkyl, C₁₋₆ substituted alkyl, C₃₋₈ cycloalkyl, C₃₋₈ substituted cycloalkyl, phenyl and benzyl, or both together with the carbon atom to which they are attached form a ring containing 3, 4, 5 or 6 carbon atoms;
R₇, R₈ and R₉ are each independently selected from the group consisting of:
H, acetyl, benzoyl; and
R₁₀ is selected from the group consisting of:
methyl, ethyl, propyl; and
m is 0 or 1 or 2; and
n is 0 or 1 or 2 or 3 or 4, wherein when n is 2, 3 or 4, each 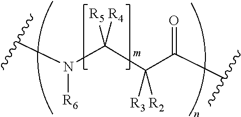
residue is independently selected; and
p is 0 or 1; and
q is 0 or 1.

2. The compound according to claim 1 wherein the ANTISENSE is a phosphorodiamidate morpholino oligonucleotide (PMO) or a peptide nucleic acid (PNA).

3. The compound according to claim 1 wherein p is 1.

4. The compound according to claim 1 wherein R₁ is selected from the group consisting of: H, Me C₁₋₆ alkyl, and C₁₋₆ substituted alkyl.

5-6. (canceled)

7. The compound according to claim 1 wherein p is 0.

8. The compound according to claim 1 wherein n is 1.

9. The compound according to claim 1 wherein one of R₂ and R₃ is H and the other is C₁₋₆ alkyl.

10. The compound according to claim 1 wherein m is 0 and R₄ and R₅ are absent.

11. The compound according to claim 1 wherein n is 2 and wherein one 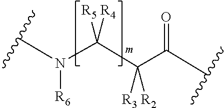
residue is 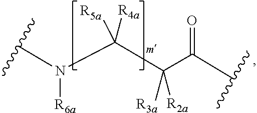
wherein R₂ₐ, R₃ₐ, R₄ₐ, R₅ₐ, R₆ₐ and m′ have the same respective definition as the moieties R₂, R₃, R₄, R₅, R₆ and m as described in claim 1.

12. The compound of claim 11 wherein one of R₂ and R₃ is H and the other is C₁₋₆ alkyl and R₂ₐ and R₃ₐ are each H.

13. The compound of claim 11 wherein m is 0, wherein m′ is 2, and wherein R₄ₐ and R₅ₐ and R₆ₐ are H.

14-16. (canceled)

17. The compound of claim 1 wherein R₆ is H.

18. The compound of claim 1 wherein q is 1.

19. The compound of claim 1 wherein L₂ has one of the following structures:

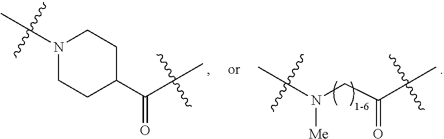

20. (canceled)

21. The compound of claim 1 wherein q is 0.

22. The compound according to claim 1 wherein n is 1, m is 0, R₆ is H, R₂ is H, R₃ is Me, p is 1, R₁ is H or Me, q is 1, and L₂ has one of the following structures:

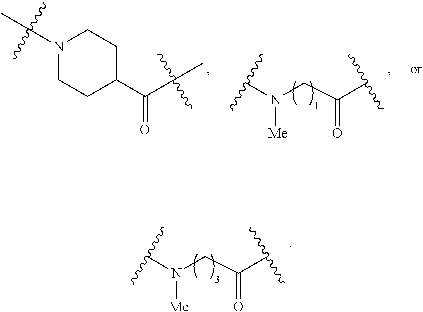

23-24. (canceled)

25. The compound according to claim 1 wherein p is 0, n is 2 and q is 0, wherein one 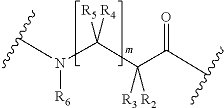
residue is 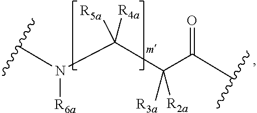
wherein R₂ₐ, R₃ₐ, R₄ₐ, R₅ₐ, R₆ₐ and m′ have the same respective definition as the moieties R₂, R₃, R₄, R₅, R₆ and m as described in claim 1, and wherein R₂ and R₃ are each independently selected from the group consisting of: H and C₁₋₆ alkyl, R₂ₐ and R₃ₐ are each H, R₄, R₅, R₄ₐ and R₅ₐ are each H, m is 0, m′ is 2, R₆ is H and R₆ₐ is H.

26. (canceled)

27. The compound according to claim 1 wherein the SUGAR is

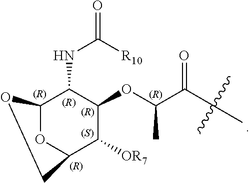

28. The compound according to any preceding claim wherein the ANTISENSE includes a sequence that is selected from the group consisting of: SEQ ID NOS: 1-172

29. (canceled)

30. A pharmaceutical or veterinary composition comprising a compound according to claim 1 and a pharmaceutically acceptable or veterinarily acceptable diluent, excipient, carrier, or combinations thereof.

31-34. (canceled)


SEQUENCE LISTING (172 sequences)

SEQ ID NO: 1 - DNA, 11 bp, Artificial Sequence
  NDM-1 targeting sequence
tcaagttttcc

SEQ ID NO: 2 - DNA, 11 bp, Artificial Sequence
  NDM-1 targeting sequence
tccttttattc

SEQ ID NO: 3 - DNA, 11 bp, Artificial Sequence
  NDM-1 targeting sequence
ccatcaagttt

SEQ ID NO: 4 - DNA, 11 bp, Artificial Sequence
  NDM-1 targeting sequence
ggcaattccat

SEQ ID NO: 5 - DNA, 11 bp, Artificial Sequence
  adeA targeting sequence
atactgtccaa

SEQ ID NO: 6 - DNA, 11 bp, Artificial Sequence
  OmpA targeting sequence
catggatatcc

SEQ ID NO: 7 - DNA, 11 bp, Artificial Sequence
  AcrA targeting sequence
atgtaaacctc

SEQ ID NO: 8 - DNA, 11 bp, Artificial Sequence
  AcrA targeting sequence
gttcatatgta

SEQ ID NO: 9 - DNA, 11 bp, Artificial Sequence
  AcrA targeting sequence
acccctctgtt

SEQ ID NO: 10 - DNA, 11 bp, Artificial Sequence
  AcrA targeting sequence
tgttcatatgt

SEQ ID NO: 11 - DNA, 11 bp, Artificial Sequence
  AcrB targeting sequence
gtcttaacggc

SEQ ID NO: 12 - DNA, 11 bp, Artificial Sequence
  AcrB targeting sequence
aggcatgtctt

SEQ ID NO: 13 - DNA, 11 bp, Artificial Sequence
  AcrB targeting sequence
taggcatgtct

SEQ ID NO: 14 - DNA, 11 bp, Artificial Sequence
  AcrR targeting sequence
tatgttcgtga

SEQ ID NO: 15 - DNA, 11 bp, Artificial Sequence
  TolC targeting sequence
ttcatttgcat

SEQ ID NO: 16 - DNA, 11 bp, Artificial Sequence
  TolC targeting sequence
attccttgtgg

SEQ ID NO: 17 - DNA, 11 bp, Artificial Sequence
  TolC targeting sequence
tttgcattcct

SEQ ID NO: 18 - DNA, 11 bp, Artificial Sequence
  KPC targeting sequence
gatacagtgac

SEQ ID NO: 19 - DNA, 11 bp, Artificial Sequence
  KPC 1-4 targeting sequence
aacgatattcc

SEQ ID NO: 20 - DNA, 11 bp, Artificial Sequence
  cepl targeting sequence
aaggtctgcat

SEQ ID NO: 21 - DNA, 11 bp, Artificial Sequence
  cepl targeting sequence
tcggatctgtg

SEQ ID NO: 22 - DNA, 11 bp, Artificial Sequence
  cepl targeting sequence
catggatgtcc

SEQ ID NO: 23 - DNA, 11 bp, Artificial Sequence
  cepl targeting sequence
cgtgaacgaag

SEQ ID NO: 24 - DNA, 11 bp, Artificial Sequence
  cepl targeting sequence
cgtgtggcaac

SEQ ID NO: 25 - DNA, 11 bp, Artificial Sequence
  cepl targeting sequence
gcccgagatcc

SEQ ID NO: 26 - DNA, 11 bp, Artificial Sequence
  cepl targeting sequence
ctttcgttcgc

SEQ ID NO: 27 - DNA, 11 bp, Artificial Sequence
  suhB targeting sequence
atgcatgagcc

SEQ ID NO: 28 - DNA, 11 bp, Artificial Sequence
  suhB targeting sequence
ggatgcatgag

SEQ ID NO: 29 - DNA, 12 bp, Artificial Sequence
  CsuE targeting sequence
ttatattcatgg

SEQ ID NO: 30 - DNA, 11 bp, Artificial Sequence
  CsuE targeting sequence
tcatggcaaag

SEQ ID NO: 31 - DNA, 11 bp, Artificial Sequence
  CsuE targeting sequence
tttcctgtcaa

SEQ ID NO: 32 - DNA, 11 bp, Artificial Sequence
  SecA targeting sequence
ttgccaacatg

SEQ ID NO: 33 - DNA, 11 bp, Artificial Sequence
  Pg1L targeting sequence
cattacccaag

SEQ ID NO: 34 - DNA, 11 bp, Artificial Sequence
  PilU1 targeting sequence
ttaaaatccat

SEQ ID NO: 35 - DNA, 11 bp, Artificial Sequence
  AlgZ targeting sequence
taggcatcgac

SEQ ID NO: 36 - DNA, 11 bp, Artificial Sequence
  AlgU targeting sequence
aaagctcctct

SEQ ID NO: 37 - DNA, 11 bp, Artificial Sequence
  LasR targeting sequence
aggccatagcg

SEQ ID NO: 38 - DNA, 11 bp, Artificial Sequence
  FleR targeting sequence
ttactcctgaa

SEQ ID NO: 39 - DNA, 11 bp, Artificial Sequence
  PelF targeting sequence
ttcggtcatgt

SEQ ID NO: 40 - DNA, 11 bp, Artificial Sequence
  acpP targeting sequence
gtccattaccc

SEQ ID NO: 41 - DNA, 11 bp, Artificial Sequence
  acpP targeting sequence
cattacccctc

SEQ ID NO: 42 - DNA, 11 bp, Artificial Sequence
  acpP targeting sequence
ccattacccct

SEQ ID NO: 43 - DNA, 11 bp, Artificial Sequence
  acpP targeting sequence
tccattacccc

SEQ ID NO: 44 - DNA, 11 bp, Artificial Sequence
  acpP targeting sequence
tgtccattacc

SEQ ID NO: 45 - DNA, 11 bp, Artificial Sequence
  acpP targeting sequence
ttgtccattac

SEQ ID NO: 46 - DNA, 11 bp, Artificial Sequence
  acpP targeting sequence
gttgtccatta

SEQ ID NO: 47 - DNA, 11 bp, Artificial Sequence
  acpP targeting sequence
tgttgtccatt

SEQ ID NO: 48 - DNA, 11 bp, Artificial Sequence
  acpP targeting sequence
atgttgtccat

SEQ ID NO: 49 - DNA, 11 bp, Artificial Sequence
  acpP targeting sequence
tttacaagtgc

SEQ ID NO: 50 - DNA, 11 bp, Artificial Sequence
  acpP targeting sequence
cctccgaggga

SEQ ID NO: 51 - DNA, 11 bp, Artificial Sequence
  acpP targeting sequence
acacgttgttc

SEQ ID NO: 52 - DNA, 11 bp, Artificial Sequence
  acpP targeting sequence
agttcagcgac

SEQ ID NO: 53 - DNA, 11 bp, Artificial Sequence
  acpP targeting sequence
ctcataccttg

SEQ ID NO: 54 - DNA, 11 bp, Artificial Sequence
  acpP targeting sequence
tgctcatactc

SEQ ID NO: 55 - DNA, 10 bp, Artificial Sequence
  acpP targeting sequence
ctcatactct

SEQ ID NO: 56 - DNA, 10 bp, Artificial Sequence
  acpP targeting sequence
ctcatactat

SEQ ID NO: 57 - DNA, 11 bp, Artificial Sequence
  acpP targeting sequence
cttcgatagtg

SEQ ID NO: 58 - DNA, 11 bp, Artificial Sequence
  acpP targeting sequence
atatcgctcac

SEQ ID NO: 59 - DNA, 11 bp, Artificial Sequence
  acpP targeting sequence
attctcctcat

SEQ ID NO: 60 - DNA, 11 bp, Artificial Sequence
  acpP targeting sequence
cacaggaattc

SEQ ID NO: 61 - DNA, 11 bp, Artificial Sequence
  acpP targeting sequence
cattgcttgtg

SEQ ID NO: 62 - DNA, 11 bp, Artificial Sequence
  acpP targeting sequence
cataccttgtt

SEQ ID NO: 63 - DNA, 11 bp, Artificial Sequence
  acpS targeting sequence
ttgccattagc

SEQ ID NO: 64 - DNA, 17 bp, Artificial Sequence
  acp-E targeting sequence
ctgtagtgatttcacca

SEQ ID NO: 65 - DNA, 11 bp, Artificial Sequence
  fabA targeting sequence
ttatctaccat

SEQ ID NO: 66 - DNA, 11 bp, Artificial Sequence
  fabB targeting sequence
cgtttcattaa

SEQ ID NO: 67 - DNA, 11 bp, Artificial Sequence
  fabB targeting sequence
gcacgtttcat

SEQ ID NO: 68 - DNA, 11 bp, Artificial Sequence
  fabl targeting sequence
agaaaacccat

SEQ ID NO: 69 - DNA, 10 bp, Artificial Sequence
  fabl targeting sequence
gctttaatcc

SEQ ID NO: 70 - DNA, 10 bp, Artificial Sequence
  fabl targeting sequence
cccatagctt

SEQ ID NO: 71 - DNA, 10 bp, Artificial Sequence
  fabl targeting sequence
catgtaagat

SEQ ID NO: 72 - DNA, 10 bp, Artificial Sequence
  fabl targeting sequence
agataactcc

SEQ ID NO: 73 - DNA, 11 bp, Artificial Sequence
  gapA targeting sequence
ttgatagtcat

SEQ ID NO: 74 - DNA, 11 bp, Artificial Sequence
  accA targeting sequence
gcttttttcat

SEQ ID NO: 75 - DNA, 11 bp, Artificial Sequence
  accA targeting sequence
aggcttccgtc

SEQ ID NO: 76 - DNA, 10 bp, Artificial Sequence
  fabD targeting sequence
gtcatgtttt

SEQ ID NO: 77 - DNA, 10 bp, Artificial Sequence
  inhA targeting sequence
gtcatttggt

SEQ ID NO: 78 - DNA, 12 bp, Artificial Sequence
  inhA targeting sequence
catttggtgact

SEQ ID NO: 79 - DNA, 11 bp, Artificial Sequence
  RpoD targeting sequence
tcatctttgct

SEQ ID NO: 80 - DNA, 11 bp, Artificial Sequence
  RpoD targeting sequence
ttttgctccat

SEQ ID NO: 81 - DNA, 11 bp, Artificial Sequence
  PolB targeting sequence
agtaactccac

SEQ ID NO: 82 - DNA, 11 bp, Artificial Sequence
  murA targeting sequence
tttatccattg

SEQ ID NO: 83 - DNA, 11 bp, Artificial Sequence
  rpsJ targeting sequence
gcatttgacct

SEQ ID NO: 84 - DNA, 11 bp, Artificial Sequence
  rpsJ targeting sequence
tagacatacca

SEQ ID NO: 85 - DNA, 11 bp, Artificial Sequence
  rpsJ targeting sequence
taccagtaaac

SEQ ID NO: 86 - DNA, 11 bp, Artificial Sequence
  rpsJ targeting sequence
tggttctgcat

SEQ ID NO: 87 - DNA, 11 bp, Artificial Sequence
  rpsJ targeting sequence
cctcagactcc

SEQ ID NO: 88 - DNA, 11 bp, Artificial Sequence
  rpsJ targeting sequence
gcatttgacct

SEQ ID NO: 89 - DNA, 11 bp, Artificial Sequence
  ftsZ targeting sequence
agtttctctcc

SEQ ID NO: 90 - DNA, 11 bp, Artificial Sequence
  ftsZ targeting sequence
gttcaaacata

SEQ ID NO: 91 - DNA, 11 bp, Artificial Sequence
  ftsZ targeting sequence
tcaaatgaggc

SEQ ID NO: 92 - DNA, 11 bp, Artificial Sequence
  ftsZ targeting sequence
aatgaggccat

SEQ ID NO: 93 - DNA, 13 bp, Artificial Sequence
  ftsZ targeting sequence
atagtttctctcc

SEQ ID NO: 94 - DNA, 11 bp, Artificial Sequence
  gyrA targeting sequence
cgctcatctaa

SEQ ID NO: 95 - DNA, 12 bp, Artificial Sequence
  gyrA targeting sequence
ctatacatagac

SEQ ID NO: 96 - DNA, 15 bp, Artificial Sequence
  gyrA targeting sequence
gccatctcggacatc

SEQ ID NO: 97 - DNA, 16 bp, Artificial Sequence
  gyrA targeting sequence
ataccaggtgttatct

SEQ ID NO: 98 - DNA, 11 bp, Artificial Sequence
  dnaB targeting sequence
ttcctgccata

SEQ ID NO: 99 - DNA, 11 bp, Artificial Sequence
  LpxC targeting sequence
tttgatcatcg

SEQ ID NO: 100 - DNA, 11 bp, Artificial Sequence
  LpxC targeting sequence
tgtttgatcat

SEQ ID NO: 101 - DNA, 11 bp, Artificial Sequence
  LpxC targeting sequence
tgtttcaccat

SEQ ID NO: 102 - DNA, 11 bp, Artificial Sequence
  LpxC targeting sequence
gttgtttgatc

SEQ ID NO: 103 - DNA, 11 bp, Artificial Sequence
  23S rRNA targeting sequence
agtgctctacc

SEQ ID NO: 104 - DNA, 11 bp, Artificial Sequence
  23S rRNA targeting sequence
gcctgttatcc

SEQ ID NO: 105 - DNA, 11 bp, Artificial Sequence
  16S rRNA targeting sequence
ccatgcagcac

SEQ ID NO: 106 - DNA, 11 bp, Artificial Sequence
  16S rRNA targeting sequence
ttgcgctcgtt

SEQ ID NO: 107 - DNA, 11 bp, Artificial Sequence
  16S rRNA targeting sequence
ggctgctggca

SEQ ID NO: 108 - DNA, 10 bp, Artificial Sequence
  fmhB targeting sequence
ccatgaaaaa

SEQ ID NO: 109 - DNA, 11 bp, Artificial Sequence
  polA targeting sequence
ttcatgcctgt

SEQ ID NO: 110 - DNA, 11 bp, Artificial Sequence
  murA targeting sequence
atccatttagt

SEQ ID NO: 111 - DNA, 11 bp, Artificial Sequence
  murA targeting sequence
catttagtttg

SEQ ID NO: 112 - DNA, 11 bp, Artificial Sequence
  murA targeting sequence
aatttatccat

SEQ ID NO: 113 - DNA, 11 bp, Artificial Sequence
  murA targeting sequence
aaatttatcca

SEQ ID NO: 114 - DNA, 11 bp, Artificial Sequence
  rpmB targeting sequence
actcgggacat

SEQ ID NO: 115 - DNA, 11 bp, Artificial Sequence
  rpmB targeting sequence
ctattctccaa

SEQ ID NO: 116 - DNA, 11 bp, Artificial Sequence
  rpmB targeting sequence
ggcagactcgg

SEQ ID NO: 117 - DNA, 11 bp, Artificial Sequence
  rpmB targeting sequence
cttagacatgg

SEQ ID NO: 118 - DNA, 11 bp, Artificial Sequence
  adk targeting sequence
atgatacgcat

SEQ ID NO: 119 - DNA, 10 bp, Artificial Sequence
  adk targeting sequence
agtgccctcc

SEQ ID NO: 120 - DNA, 11 bp, Artificial Sequence
  infA targeting sequence
tctttggccat

SEQ ID NO: 121 - DNA, 11 bp, Artificial Sequence
  aroC targeting sequence
tttccagccat

SEQ ID NO: 122 - DNA, 11 bp, Artificial Sequence
  aroC targeting sequence
ttccctgccat

SEQ ID NO: 123 - DNA, 11 bp, Artificial Sequence
  murF targeting sequence
acgctaatcat

SEQ ID NO: 124 - DNA, 11 bp, Artificial Sequence
  murF targeting sequence
acctcccaggc

SEQ ID NO: 125 - DNA, 11 bp, Artificial Sequence
  kdtA targeting sequence
aattcgagcat

SEQ ID NO: 126 - DNA, 11 bp, Artificial Sequence
  boxA targeting sequence
tgtttaagagc

SEQ ID NO: 127 - DNA, 11 bp, Artificial Sequence
  boxA targeting sequence
ctcttaatgat

SEQ ID NO: 128 - DNA, 11 bp, Artificial Sequence
  boxC targeting sequence
atccaccacag

SEQ ID NO: 129 - DNA, 15 bp, Artificial Sequence
  rpoD-E targeting sequence
cttgtaaccacacca

SEQ ID NO: 130 - DNA, 15 bp, Artificial Sequence
  rpoD-E targeting sequence
tccaccaagtcacca

SEQ ID NO: 131 - DNA, 11 bp, Artificial Sequence
  pryC targeting sequence
ggtgcagtcat

SEQ ID NO: 132 - DNA, 11 bp, Artificial Sequence
  pryC targeting sequence
agagttcaagg

SEQ ID NO: 133 - DNA, 11 bp, Artificial Sequence
  pryA targeting sequence
gacttaatcaa

SEQ ID NO: 134 - DNA, 11 bp, Artificial Sequence
  lgt targeting sequence
ctactggtcat

SEQ ID NO: 135 - DNA, 11 bp, Artificial Sequence
  folA targeting sequence
cattgagattt

SEQ ID NO: 136 - DNA, 11 bp, Artificial Sequence
  infB targeting sequence
acatctgtcat

SEQ ID NO: 137 - DNA, 11 bp, Artificial Sequence
  nrdA targeting sequence
ttctgattcat

SEQ ID NO: 138 - DNA, 11 bp, Artificial Sequence
  nrdB targeting sequence
gtatatgccat

SEQ ID NO: 139 - DNA, 11 bp, Artificial Sequence
  zipA targeting sequence
tcctgcatcat

SEQ ID NO: 140 - DNA, 11 bp, Artificial Sequence
  caoA targeting sequence
atatacctcat

SEQ ID NO: 141 - DNA, 17 bp, Artificial Sequence
  gyrA-E targeting sequence
gttaccctgaccgacca

SEQ ID NO: 142 - DNA, 16 bp, Artificial Sequence
  gyrA-E targeting sequence
gttaccctgaccacca

SEQ ID NO: 143 - DNA, 11 bp, Artificial Sequence
  mrdA targeting sequence
tgtttcatacg

SEQ ID NO: 144 - DNA, 11 bp, Artificial Sequence
  LpxB targeting sequence
ggtttgccaag

SEQ ID NO: 145 - DNA, 11 bp, Artificial Sequence
  LpxB targeting sequence
taatccgtcag

SEQ ID NO: 146 - DNA, 11 bp, Artificial Sequence
  carA targeting sequence
ggtgctcaaac

SEQ ID NO: 147 - DNA, 11 bp, Artificial Sequence
  adeA targeting sequence
atactgtaaaa

SEQ ID NO: 148 - DNA, 11 bp, Artificial Sequence
  blaT targeting sequence
ctcttcctttt

SEQ ID NO: 149 - DNA, 11 bp, Artificial Sequence
  crml targeting sequence
tccttctgatt

SEQ ID NO: 150 - DNA, 10 bp, Artificial Sequence
  folP targeting sequence
atgttatccc

SEQ ID NO: 151 - DNA, 10 bp, Artificial Sequence
  fmhB targeting sequence
ccatgattta

SEQ ID NO: 152 - DNA, 10 bp, Artificial Sequence
  hmrB targeting sequence
tccacgtcga

SEQ ID NO: 153 - DNA, 11 bp, Artificial Sequence
  rpmB targeting sequence
gtctattctcc

SEQ ID NO: 154 - DNA, 11 bp, Artificial Sequence
  rpmB targeting sequence
gacatgtctat

SEQ ID NO: 155 - DNA, 10 bp, Artificial Sequence
  hmrB targeting sequence
tccacgtcga

SEQ ID NO: 156 - DNA, 11 bp, Artificial Sequence
  FabG targeting sequence
ttctctccttt

SEQ ID NO: 157 - DNA, 11 bp, Artificial Sequence
  RpmB targeting sequence
ctctagacatg

SEQ ID NO: 158 - DNA, 11 bp, Artificial Sequence
  WaaC targeting sequence
agcaccctcat

SEQ ID NO: 159 - DNA, 11 bp, Artificial Sequence
  MryA targeting sequence
tgactctcctc

SEQ ID NO: 160 - DNA, 11 bp, Artificial Sequence
  MurC targeting sequence
ccacctccagg

SEQ ID NO: 161 - DNA, 11 bp, Artificial Sequence
  LpxA targeting sequence
atcaaactcat

SEQ ID NO: 162 - DNA, 11 bp, Artificial Sequence
  WaaG targeting sequence
gccagggtcat

SEQ ID NO: 163 - DNA, 11 bp, Artificial Sequence
  WaaA targeting sequence
gtacggttcat

SEQ ID NO: 164 - DNA, 11 bp, Artificial Sequence
  murB targeting sequence
cagtcgcccct

SEQ ID NO: 165 - DNA, 11 bp, Artificial Sequence
  murE targeting sequence
aggctcatagg

SEQ ID NO: 166 - DNA, 11 bp, Artificial Sequence
  AccB targeting sequence
ctagcactccc

SEQ ID NO: 167 - DNA, 11 bp, Artificial Sequence
  FabZ targeting sequence
atgtccatcat

SEQ ID NO: 168 - DNA, 11 bp, Artificial Sequence
  MurG targeting sequence
gcaaagtcctc

SEQ ID NO: 169 - DNA, 11 bp, Artificial Sequence
  AmpR targeting sequence
gtcgaaccaat

SEQ ID NO: 170 - DNA, 12 bp, Artificial Sequence
  LepB targeting sequence
attgagtgtcat

SEQ ID NO: 171 - DNA, 12 bp, Artificial Sequence
  LptD targeting sequence
tgccatcttgtt

SEQ ID NO: 172 - DNA, 12 bp, Artificial Sequence
  MraY targeting sequence
caggagcattag